(19) United States
(12) Patent Application Publication
(10) Pub. No.: US 2024/0358482 A1
(43) Pub. Date: Oct. 31, 2024

(54) DETERMINING 3D DATA FOR 2D POINTS IN INTRAORAL SCANS

(71) Applicant: Align Technology, Inc., San Jose, CA (US)

(72) Inventors: Eliran Dafna, Beer-Sheva (IL); Ofer Saphier, Rechovot (IL)

(21) Appl. No.: 18/645,346

(22) Filed: Apr. 24, 2024

Related U.S. Application Data
(60) Provisional application No. 63/461,804, filed on Apr. 25, 2023.

(51) Int. Cl. A61C 9/00; A61B 1/00; A61B 1/05; A61B 1/06; A61B 1/24; G06T 7/00; G06T 7/73; G06T 7/90; H04N 23/11; H04N 23/56; H04N 23/90

(52) U.S. Cl. CPC A61C 9/006; A61B 1/000096; A61B 1/051; A61B 1/0615; A61B 1/0625; A61B 1/24; G06T 7/0012; G06T 7/73; G06T 7/90; H04N 23/11; H04N 23/56; H04N 23/90; G06T 2207/10024; G06T 2207/10048; G06T 2207/10152; G06T 2207/20076; G06T 2207/30036

(57) ABSTRACT

Embodiments relate to techniques for determining 3D data for 2D points in 2D images using machine learning. A method comprises using one or more trained machine learning models to determine correspondence between captured points of a captured light pattern in one or more images and projected points of a projected light pattern and determining depth information for at least some of the plurality of captured points based on the determined correspondence.

### RELATED APPLICATIONS

The present application claims priority to U.S. Provisional Patent Application No. 63/461,804, filed on Apr. 25, 2023, which is herein incorporated by reference in its entirety.

### TECHNICAL FIELD

Embodiments of the present disclosure relate to the field of dentistry and, in particular, to systems and methods for determining three-dimensional (3D) data for 2D points in intraoral images.

### BACKGROUND

Dental impressions of a subject's intraoral 3D surface, e.g., teeth and gingiva, are used for planning dental procedures. Traditional dental impressions are made using a dental impression tray filled with an impression material, e.g., PVS or alginate, into which the subject bites. The impression material then solidifies into a negative imprint of the teeth and gingiva, from which a 3D model of the teeth and gingiva can be formed.

Digital dental impressions utilize intraoral scanning to generate 3D digital models of an intraoral 3D surface of a subject. Digital intraoral scanners often use structured light 3D imaging. The surface of a subject's teeth may be highly reflective and somewhat translucent, which may reduce the contrast in the structured light pattern reflecting off the teeth. Therefore, in order to improve the capture of an intraoral scan, when using a digital intraoral scanner that utilizes structured light 3D imaging, a subject's teeth are frequently coated with an opaque powder prior to scanning in order to facilitate a usable level of contrast of the structured light pattern, e.g., in order to turn the surface into a scattering surface. While intraoral scanners utilizing structured light 3D imaging have made some progress, additional advantages may be had.

### SUMMARY

A few example implementations are summarized. These example implementations should not be construed as limiting.

In a first implementation, a method comprises: projecting, by one or more structured light projectors of an intraoral scanner, a light pattern comprising a plurality of projector rays onto a dental site; capturing, by a plurality of cameras of the intraoral scanner, a plurality of images of at least a portion of the light pattern projected onto the dental site, wherein each camera of the plurality of cameras captures an image of the plurality of images, the image comprising a plurality of points of at least the portion of the light pattern projected onto the dental site; determining, for each projector ray of the plurality of projector rays, one or more candidate points of the plurality of points that might have been caused by the projector ray; processing information for each projector ray using a trained machine learning model, wherein the trained machine learning model generates one or more outputs comprising, for each projector ray, and for each candidate point associated with the projector ray, a probability that the candidate point corresponds to the projector ray; and determining three-dimensional (3D) coordinates for at least some of the plurality of points in the plurality of images based on the one or more outputs of the trained machine learning model.

A second implementation may further extend the first implementation. In the second implementation, each of the plurality of images is a two-dimensional (2D) image.

A third implementation may further extend any of the first or second implementations. In the third implementation, the method further comprises: determining, for each projector ray, and for each candidate point of the one or more candidate points that might have been caused by the projector ray, a distance at which the candidate point intersects with the projector ray, wherein the information for the projector ray that is input into the trained machine learning model comprises the distance.

A fourth implementation may further extend the third implementation. In the fourth implementation, the method further comprises: for each projector ray, grouping one or more candidate points from different images of the plurality of images for which the distance matches into a candidate intersection, wherein the candidate intersection comprises an intersection of the one or more candidate points with the projector ray.

A fifth implementation may further extend the fourth implementation. In the fifth implementation, the distance for candidate points match if the distance varies by less than a threshold amount.

A sixth implementation may further extend the fourth or fifth implementation. In the sixth implementation, the method further comprises: determining, for each candidate intersection, a triangulation point of the candidate intersection, wherein the information for the projector ray that is input into the trained machine learning model comprises the triangulation point.

A seventh implementation may further extend any of the third through or sixth implementations. In the seventh implementation, the one or more structured light projectors comprise a plurality of structured light projectors, the method further comprising: determining, for each projector ray, an index of a structured light projector of the plurality of structured light projectors that generated the projector ray, wherein the information for the projector ray that is input into the trained machine learning model comprises the index.

An eighth implementation may further extend the seventh implementation. In the eighth implementation, a first subset of the plurality of structured light projectors produces light having a first wavelength, and wherein a second subset of the plurality of structured light projectors produces light having a second wavelength, the method further comprising: determining the 3D coordinates for one or more points of a first subset of points of the plurality of points having the first wavelength; and independently determining the 3D coordinates for one or more additional points of a second subset of points of the plurality of points having the second wavelength.

A ninth implementation may further extend the eighth implementation. In the ninth implementation, the method further comprises: identifying one or more projector rays for which candidate points from at least one of the first subset of points or the second subset of points have not been selected; combining information for the first subset of points and the second subset of points; and determining the 3D coordinates for one or more additional points of the first subset of points and the 3D coordinates for one or more additional points of the second subset of points after combining the information.

A 10^{th} implementation may further extend any of the third through 9^{th} implementations. In the 10^{th} implementation, the method further comprises: determining, for each projector ray, and for one or more candidate points associated with the projector ray, one or more properties associated with the projector ray and the one or more candidate points, wherein the information for the projector ray that is input into the trained machine learning model comprises the one or more properties.

An 11^{th} implementation may further extend the 10^{th} implementation. In the 11^{th} implementation, the one or more properties comprise a distance from an epi-polar line associated with the projector ray.

A 12^{th} implementation may further extend the 11^{th} implementation. IN the 12^{th} implementation, the distance from the epi-polar line comprises an orthogonal distance from the epi-polar line.

A 13^{th} implementation may further extend any of the 10^{th} through 12^{th} implementations. In the 13^{th} implementation, the one or more properties comprise, for an image associated with a candidate point, a triangulation error that is determined based on a distance between a camera that captured the image and an origin of the projector ray.

A 14^{th} implementation may further extend any of the 10^{th} through 13^{th} implementations. In the 14^{th} implementation, the one or more properties comprise an intensity associated with the captured point.

A 15^{th} implementation may further extend any of the 10^{th} through 14^{th} implementations. In the 15^{th} implementation, the captured point comprises a captured spot, and wherein the one or more properties comprise a spot size of the captured spot.

A 16^{th} implementation may further extend any of the 10^{th} through 15^{th} implementations. In the 16^{th} implementation, the one or more properties comprise a color of the dental site at the intersection of a candidate point with the projector ray as determined from one or more color images captured at least one of before or after capture of the plurality of images.

A 17^{th} implementation may further extend any of the 1^{st} through 16^{th} implementations. In the 17^{th} implementation, the method further comprises: generating a tuple for a projector ray comprising: distances and probabilities for one or more top candidate points for the projector ray; and distances and probabilities for one or more top candidate points for one or more additional projector rays that are proximate to the projector ray; and inputting the tuple into a second trained machine learning model, wherein the second trained machine learning model outputs an updated probability for one or more candidate points for the projector ray.

An 18^{th} implementation may further extend any of the 1^{st} through 17^{th} implementations. In the 18^{th} implementation, the plurality of images are associated with a current frame, and wherein a previous plurality of images was generated at a prior frame prior to generation of the plurality of images, the method further comprising: determining, for a projector ray, a 3D coordinate associated with the projector ray for the prior frame; and updating, for a candidate point for the projector ray, the probability that the candidate point corresponds to the projector ray based on the 3D coordinate associated with the projector ray for the prior frame.

A 19^{th} implementation may further extend any of the 1^{st} through 18^{th} implementations. In the 19^{th} implementation, the method further comprises: using a second trained machine learning model to select candidate points for a plurality of projector rays based on one or more inputs comprising probabilities of candidate points corresponding to projector rays, wherein the 3D coordinates are determined based on the selected candidate points.

A 20^{th} implementation may further extend any of the 1^{st} through 19^{th} implementations. In the 20^{th} implementation, a computer readable medium comprises instructions that, when executed by a processing device, cause the processing device to perform the method of any of the 1^{st} through 19^{th} implementations.

A 21^{st} implementation may further extend any of the 1^{st} through 19^{th} implementations. In the 21^{st} implementation, an intraoral scanning system comprises: the intraoral scanner to generate the plurality of images; and a computing device, wherein the computing device is to perform the method of any of the 1^{st} through 19^{th} implementations.

In a 22^{nd} implementation, a method comprises: projecting, by one or more structured light projectors of an intraoral scanner, a light pattern comprising a plurality of projector rays onto a dental site; capturing, by a plurality of cameras of the intraoral scanner, a plurality of images of at least a portion of the light pattern projected onto the dental site, wherein each camera of the plurality of cameras captures an image of the plurality of images, the image comprising a plurality of points of at least the portion of the light pattern projected onto the dental site; determining, for each projector ray of the plurality of projector rays, one or more candidate points of the plurality of points that might have been caused by the projector ray, each candidate point of the one or more candidate points having a determined probability of corresponding to the projector ray; using a trained machine learning model to select candidate points for a plurality of projector rays based on one or more inputs comprising probabilities of candidate points corresponding to projector rays; and determining three-dimensional (3D) coordinates for at least some of the plurality of points in the plurality of images based on the selected candidate points for the plurality of projector rays.

A 23^{rd} implementation may further extend the 22^{nd} implementation. In the 23^{rd} implementation, the method further comprises: generating an input comprising a candidate point for a projector ray, one or more additional candidate points for the projector ray, and one or more additional projector rays for the candidate point; and providing the input to the trained machine learning model, wherein the trained machine learning model outputs a selection of the candidate point or one of the one or more additional candidate points for the projector ray.

A 24^{th} implementation may further extend the 23^{rd} implementation. In the 24^{th} implementation, the method further comprises: removing the selected candidate point from association with the one or more additional projector rays that were associated with the selected candidate point.

A 25^{th} implementation may further extend the 24^{th} implementation. In the 25^{th} implementation, the method further comprises: generating a next input comprising a next candidate point for a next projector ray, one or more next additional candidate points for the next projector ray, and one or more next additional projector rays for the next candidate point; providing the next input to the trained machine learning model, wherein the trained machine learning model outputs a selection of the next candidate point or one of the one or more next additional candidate points for the next projector ray; and removing the selected next candidate point from association with the one or more next additional projector rays that were associated with the selected next candidate point.

A 26^{th} implementation may further extend the 25^{th} implementation. In the 26^{th} implementation, the method further comprises: repeating the generating of the next input, the providing of the next input to the trained machine learning model, and the removing of the selected next candidate point for a plurality of next additional projector rays until no remaining projector rays have an associated candidate point with at least a threshold probability.

A 27^{th} implementation may further extend the 26^{th} implementation. In the 27^{th} implementation, the method further comprises: reducing the threshold probability to a second threshold probability; and repeating the generating of the next input, the providing of the next input to the trained machine learning model, and the removing of the selected next candidate point for a plurality of additional projector rays until no remaining projector rays have an associated candidate point with at least the second threshold probability.

A 28^{th} implementation may further extend any of the 23^{rd} through 27^{th} implementations. In the 28^{th} implementation, the method further comprises: generating a first list associating projector rays with candidate intersections, wherein each candidate intersection comprises an intersection of a projector ray of the plurality of projector rays and a candidate point of the one or more candidate points that might have been caused by the projector ray, the first list comprising, for each projector ray, one or more candidate intersections associated with the projector ray; and generating a second list of the plurality of points, the second list comprising, for each point of the plurality of points, one or more candidate intersections associated with the point; wherein at least one of the first list or the second list is used to generate the input.

A 29^{th} implementation may further extend any of the 22^{nd} through 28^{th} implementations. In the 29^{th} implementation, a computer readable medium comprises instructions that, when executed by a processing device, cause the processing device to perform the method of any of the 22^{nd} through 28^{th} implementations.

A 30^{th} implementation may further extend any of the 22^{nd} through 28^{th} implementations. In the 30^{th} implementation, an intraoral scanning system comprises: the intraoral scanner to generate the plurality of images; and a computing device, wherein the computing device is to perform the method of any of the 22^{nd} through 28^{th} implementations.

In a 31^{st} implementation, a method comprises: using a first trained machine learning model to determine probabilities that captured points of a captured light pattern in one or more images correspond to projected points of a projected light pattern; using a second trained machine learning model to determine correspondence between a plurality of the captured points and a plurality of the projected points based on one or more of the determined probabilities; and determining depth information for at least some of the plurality of captured points based on the determined correspondence.

A 32^{nd} implementation may further extend the 31^{st} implementation. In the 32^{nd} implementation, a computer readable medium comprises instructions that, when executed by a processing device, cause the processing device to perform the method of the 31^{st} implementation.

A 33^{rd} implementation may further extend the 31^{st} implementation. In the 33^{rd} implementation, an intraoral scanning system comprises: the intraoral scanner to generate the plurality of images; and a computing device, wherein the computing device is to perform the method of the 31^{st} implementation.

In a 34^{th} implementation, a method comprises: using one or more trained machine learning models to determine correspondence between captured points of a captured light pattern in one or more images and projected points of a projected light pattern; and determining depth information for at least some of the plurality of captured points based on the determined correspondence.

A 35^{th} implementation may further extend the 34^{th} implementation. In the 35^{th} implementation, a computer readable medium comprises instructions that, when executed by a processing device, cause the processing device to perform the method of the 34^{th} implementation.

A 36^{th} implementation may further extend the 34^{th} implementation. In the 36^{th} implementation, an intraoral scanning system comprises: the intraoral scanner to generate the plurality of images; and a computing device, wherein the computing device is to perform the method of the 34^{th} implementation.

In a 37^{th} implementation, a method comprises: projecting, by one or more structured light projectors of an intraoral scanner, a light pattern comprising a plurality of projector rays onto a dental site, wherein the plurality of projector rays form a plurality of features of the light pattern; capturing, by a plurality of cameras of the intraoral scanner, a plurality of images of at least a portion of the light pattern projected onto the dental site, wherein each camera of the plurality of cameras captures an image of the plurality of images, the image comprising a subset of the plurality of features; determining, for each projector ray of the plurality of projector rays, one or more candidate points of the plurality of features that might have been caused by the projector ray, each combination of a candidate point and a projector ray corresponding to a candidate intersection; processing information for each candidate intersection to determine a probability that the candidate point of the candidate intersection corresponds to the projector ray of the candidate intersection; selecting a subset of candidate intersections based at least in part on determined probabilities; and determining three-dimensional (3D) coordinates for at least some of the plurality of features according to the selected subset of the candidate intersections.

A 38^{th} implementation may further extend the 37^{th} implementation. In the 38^{th} implementation, processing the information for a projector ray is performed using a trained machine learning model, wherein the trained machine learning model generates one or more outputs comprising, for each candidate intersection, the probability that the candidate point of the candidate intersection corresponds to the projector ray of the candidate intersection.

A 39^{th} implementation may further extend any of the 37^{th} through 38^{th} implementations. In the 39^{th} implementation, the selecting the subset of the candidate intersections is performed using a trained machine learning model based on one or more inputs comprising the determined probabilities.

A 40^{th} implementation may further extend any of the 37^{th} through 39^{th} implementations. In the 40^{th} implementation, each of the plurality of images is a two-dimensional (2D) image.

A 41^{st} implementation may further extend any of the 37^{th} through 40^{th} implementations. In the 41^{st} implementation, the light pattern comprises a pattern of spots, and wherein the plurality of features comprises a plurality of discrete unconnected spots.

A 42^{nd} implementation may further extend any of the 37^{th} through 40^{th} implementations. In the 42^{nd} implementation, the light pattern comprises a checkerboard pattern, and wherein the plurality of features comprise a plurality of regions of the checkerboard pattern.

A 43^{rd} implementation may further extend any of the 37^{th} through 42^{nd} implementations. In the 43^{rd} implementation, the processing the information is performed using a first trained machine learning model, and wherein the selecting the subset of the candidate intersections is performed using a second trained machine learning model.

A 44^{th} implementation may further extend any of the 37^{th} through 43^{rd} implementations. In the 44^{th} implementation, the method further comprises: determining, for a feature of the plurality of features, that no projector ray of the plurality of projector rays has a candidate intersection for the feature with a probability that meets a probability threshold; and removing the feature from consideration.

A 45^{th} implementation may further extend any of the 37^{th} through 44^{th} implementations. In the 45^{th} implementation, the method further comprises: determining, for a projector ray of the plurality of projector rays, that no candidate intersection associated with the projector ray has a probability that meets a probability threshold; and removing the projector ray from consideration, wherein no 3D coordinate is determined for the projector ray.

A 46^{th} implementation may further extend any of the 37^{th} through 45^{th} implementations. In the 46^{th} implementation, the method further comprises: determining, for a projector ray of the plurality of projector rays, that a first candidate intersection associated with the projector ray has a first probability, that a second candidate intersection associated with the projector ray has a second probability, and that a delta between the first probability and the second probability is less than a threshold; and removing the projector ray from consideration, wherein no 3D coordinate is determined for the projector ray.

A 47^{th} implementation may further extend the 46^{th} implementation. In the 47^{th} implementation, the first probability and the second probability are each at or above a probability threshold.

A 48^{th} implementation may further extend any of the 37^{th} through 47^{th} implementations. In the 48^{th} implementation, at most one candidate intersection is determined for each projector ray of the plurality of projector rays.

A 49^{th} implementation may further extend any of the 37^{th} through 48^{th} implementations. In the 49^{th} implementation, the plurality of projector rays are arranged in a known grid pattern (e.g., in a known order), the method further comprising: determining, for one or more remaining projector rays for which a candidate intersection has not been selected, one or more candidate intersections that fail to preserve the known order; and removing the one or more candidate intersections from consideration.

A 50^{th} implementation may further extend the 49^{th} implementation. In the 50^{th} implementation, the method further comprises: determining, for each remaining projector ray of the one or more remaining projector rays, and for each remaining candidate intersection associated with the remaining projector ray, an updated probability that the remaining candidate intersection corresponds to the remaining projector ray

A 51^{st} implementation may further extend any of the 37^{th} through 50^{th} implementations. In the 51^{st} implementation, the plurality of projector rays are arranged in a first known order along a first axis and in a second known order along a second axis, the method further comprising: determining, for one or more remaining projector rays for which a candidate intersection has not been selected, one or more candidate intersections that fail to preserve at least one of the first known order or the second known order; and removing the one or more candidate intersections from consideration.

A 52^{nd} implementation may further extend the 37^{th} through 51^{st} implementations. In the 52^{nd} implementation, the plurality of projector rays are arranged in a hexagonal grid pattern.

A 53^{rd} implementation may further extend the 52^{nd} implementation. In the 53^{rd} implementation, the method further comprises: performing an affine transformation to at least one of the plurality of projector rays or the plurality of features to transform the hexagonal grid pattern into a rectangular grid pattern, wherein the first axis and the second axis are axes of the rectangular grid pattern.

A 54^{th} implementation may further extend any of the 37^{th} through 53^{rd} implementations. In the 54^{th} implementation, a computer readable medium comprises instructions that, when executed by a processing device, cause the processing device to perform the method of any of the 37^{th} through 53^{rd} implementations.

A 55^{th} implementation may further extend any of the 37^{th} through 53^{rd} implementations. In the 55^{th} implementation, an intraoral scanning system comprises: the intraoral scanner to generate the plurality of images; and a computing device, wherein the computing device is to perform the method of any of the 37^{th} through 53^{rd} implementations.

In a 56^{th} implementation, a method comprises: receiving probabilities relating structured light features in one or more images captured by one or more cameras of an intraoral scanner with projector rays of a light pattern projected by one or more structured light projectors of the intraoral scanner; determining three-dimensional (3D) coordinates of a subset of the structured light features by associating the subset of the structured light features with a subset of the projector rays based on the received probabilities; constraining projector ray candidates for non-associated structured light features by removing one or more projector ray candidates for non-associated structured light features that do not preserve order with the subset of the structured light features associated with the subset of the projector rays; and solving, after constraining the projector ray candidates for the non-associated structured light features, for 3D coordinates of at least a subset of the non-associated structured light features by associating at least the subset of the non-associated structured light features with a subset of the non-associated projector rays.

A 57^{th} implementation may further extend the 56^{th} implementation. In the 57^{th} implementation, each of the one or more images is a two-dimensional (2D) image.

A 58^{th} implementation may further extend any of the 56^{th} through 57^{th} implementations. In the 58^{th} implementation, the light pattern comprises a pattern of spots, and wherein the structured light features comprise a plurality of discrete unconnected spots.

A 59^{th} implementation may further extend any of the 56^{th} through 57^{th} implementations. In the 58^{th} implementation, the light pattern comprises a checkerboard pattern, and wherein the structured light features comprise a plurality of regions of the checkerboard pattern.

A 60^{th} implementation may further extend any of the 56^{th} through 59^{th} implementations. In the 60^{th} implementation, the method further comprises: determining, for a feature of the structured light features, that no projector ray candidate has a probability associating the projector ray candidate with the feature that meets a probability threshold; and removing the feature from consideration.

A 61^{st} implementation may further extend any of the 56^{th} through 60^{th} implementations. In the 61^{st} implementation, the method further comprises: determining, for a feature of the structured light features, that a first projector ray candidate has a first probability associating the first projector ray candidate with the feature, that a second projector ray candidate has a second probability associating the second projector ray candidate with the feature, and that a delta between the first probability and the second probability is less than a threshold; and removing the feature from consideration, wherein no 3D coordinate is solved for the feature.

A 62^{nd} implementation may further extend the 61^{st} implementation. In the 62^{nd} implementation, the first probability and the second probability are each at or above a probability threshold.

A 63^{rd} implementation may further extend any of the 56^{th} through 62^{nd} implementations. In the 63^{rd} implementation, at most one projector ray is associated with each structured light feature.

A 64^{th} implementation may further extend any of the 56^{th} through 63^{rd} implementations. In the 64^{th} implementation, the method further comprises: determining updated probabilities associating non-associated structured light features in the one or more images with non-associated projector rays of the light pattern responsive to constraining the projector ray candidates.

A 65^{th} implementation may further extend any of the 56^{th} through 64^{th} implementations. In the 65^{th} implementation, the projector rays are arranged in a hexagonal grid pattern.

A 66^{th} implementation may further extend any of the 56^{th} through 65^{th} implementations. In the 66^{th} implementation, the method further comprises: performing an affine transformation to at least one of the projector rays or the structured light features to transform the hexagonal grid pattern into a rectangular grid pattern, wherein the constraining is performed after the affine transformation.

A 67^{th} implementation may further extend any of the 56^{th} through 66^{rd} implementations. In the 67^{th} implementation, a computer readable medium comprises instructions that, when executed by a processing device, cause the processing device to perform the method of any of the 56^{th} through 66^{rd} implementations.

A 68^{th} implementation may further extend any of the 56^{th} through 66^{rd} implementations. In the 68^{th} implementation, an intraoral scanning system comprises: the intraoral scanner to generate the plurality of images; and a computing device, wherein the computing device is to perform the method of any of the 56^{th} through 66^{rd} implementations.

In a 69^{th} implementation, a method comprises: receiving candidate pairings of structured light features in one or more images captured by one or more cameras of an intraoral scanner with projector rays of a light pattern projected by one or more structured light projectors of the intraoral scanner, each of the candidate pairings comprising a probability that a structured light feature corresponds to a projector ray; removing structured light features from consideration that have no candidate pairings with probabilities that are at or above a threshold; and solving for 3D coordinates of at least a subset of the structured light features by selecting candidate pairings for the subset of the structured light features based at least in part on the probabilities.

A 70^{th} implementation may further extend the 69^{th} implementation. In the 70^{th} implementation, the method further comprises: determining, for a feature of the structured light features, that a first candidate pairing has a first probability associating a first projector ray with the feature, that a second candidate pairing has a second probability associating a second projector ray with the feature, and that a delta between the first probability and the second probability is less than a threshold; and removing the feature from consideration, wherein no 3D coordinate is solved for the feature.

A 71^{st} implementation may further extend any of the 69^{th} through 70^{th} implementations. In the 71^{st} implementation, a computer readable medium comprises instructions that, when executed by a processing device, cause the processing device to perform the method of any of the 69^{th} through 70^{th} implementations.

A 72^{nd} implementation may further extend any of the 69^{th} through 70^{th} implementations. In the 72^{nd} implementation, an intraoral scanning system comprises: the intraoral scanner to generate the plurality of images; and a computing device, wherein the computing device is to perform the method of any of the 69^{th} through 70^{th} implementations.

In a 73^{rd} implementation, a method comprises: determining candidate pairings of structured light features in one or more images captured by one or more cameras of an intraoral scanner with projector rays of a light pattern projected by one or more structured light projectors of the intraoral scanner; determining, for each candidate pairing of the candidate pairings, a probability that a structured light feature of the candidate pairing corresponds to a projector ray of the candidate pairing; and determining 3D coordinates of at least a subset of the structured light features by selecting candidate pairings based at least in part on determined probabilities.

A 74^{th} implementation may further extend the 73^{rd} implementation. In the 74^{th} implementation, the method further comprises: removing one or more candidate pairings for which a known order of structured light features is not preserved; and solving for 3D coordinates of one or more additional structured light features by selecting one or more remaining candidate pairings for the one or more structured light features.

A 75^{th} implementation may further extend any of the 73^{rd} through 74^{th} implantations. In the 75^{th} implementation, the method further comprises: removing structured light features from consideration that have no candidate pairings with probabilities that are at or above a threshold.

A 76^{th} implementation may further extend any of the 73^{rd} through 75^{th} implantations. In the 76^{th} implementation, the method further comprises: determining, for a feature of the structured light features, that a first candidate pairing has a first probability associating a first projector ray with the feature, that a second candidate pairing has a second probability associating a second projector ray with the feature, and that a delta between the first probability and the second probability is less than a threshold; and removing the feature from consideration, wherein no 3D coordinate is solved for the feature.

A 77^{th} implementation may further extend any of the 73^{rd} through 76^{th} implantations. In the 77^{th} implementation, determining a probability that a structured light feature of a candidate pairing corresponds to a projector ray of the candidate pairing comprises: processing information for the candidate pairing using a trained machine learning model, wherein the trained machine learning model generates an output comprising the probability that the structured light feature corresponds to the projector ray.

A 78^{th} implementation may further extend any of the 73^{rd} through 77^{th} implantations. In the 78^{th} implementation, selecting the candidate pairings based at least in part on the determined probabilities comprises processing one or more inputs comprising one or more of the determined probabilities using a trained machine learning model, wherein the trained machine learning model outputs one or more selections of candidate pairings.

A 79^{th} implementation may further extend any of the 73^{rd} through 78^{th} implantations. In the 79^{th} implementation, each of the one or more images is a two-dimensional (2D) image.

An 80^{th} implementation may further extend any of the 73^{rd} through 78^{th} implementations. In the 80^{th} implementation, a computer readable medium comprises instructions that, when executed by a processing device, cause the processing device to perform the method of any of the 73^{rd} through 78^{th} implementations.

An 81^{st} implementation may further extend any of the 73^{rd} through 78^{th} implementations. In the 81^{st} implementation, an intraoral scanning system comprises: the intraoral scanner to generate the plurality of images; and a computing device, wherein the computing device is to perform the method of any of the 73^{rd} through 78^{th} implementations.

### BRIEF DESCRIPTION OF THE DRAWINGS

Embodiments of the present disclosure are illustrated by way of example, and not by way of limitation, in the figures of the accompanying drawings.

FIG. 1 illustrates one embodiment of a system for performing intraoral scanning and/or generating a virtual three-dimensional model of a dental site.

FIG. 2 is a schematic illustration of an intraoral scanner with a plurality cameras disposed within a probe at a distal end of the intraoral scanner, in accordance with some applications of the present disclosure.

FIGS. 3A-3B comprise schematic illustrations of positioning configurations for cameras and structured light projectors of an intraoral scanner, in accordance with some applications of the present disclosure.

FIG. 4 is a chart depicting a plurality of different configurations for the position of structured light projectors and cameras in a probe of an intraoral scanner, in accordance with some applications of the present disclosure.

FIG. 5 is a schematic illustration of a structured light projector projecting a distribution of discrete unconnected spots of light onto a plurality of object focal planes, in accordance with some applications of the present disclosure.

FIGS. 6A-B are schematic illustrations of a structured light projector projecting discrete unconnected spots and a camera sensor detecting spots, in accordance with some applications of the present invention.

FIG. 7 is a flow chart for a method of generating a digital three-dimensional image, in accordance with embodiments of the present disclosure.

FIGS. 8-9 are schematic illustrations of a structured light projector projecting discrete unconnected spots and multiple camera sensors detecting spots, in accordance with some applications of the present disclosure.

FIG. 10 is a flow chart for a method of determining 3D coordinates for points in one or more 2D images, in accordance with embodiments of the present disclosure.

FIG. 11 is a flow chart for another method of determining 3D coordinates for points in one or more 2D images, in accordance with embodiments of the present disclosure.

FIG. 12A is a flow chart for another method of determining 3D coordinates for points in one or more 2D images, in accordance with embodiments of the present disclosure.

FIG. 12B is a flow chart for another method of determining 3D coordinates for points in one or more 2D images using machine learning, in accordance with embodiments of the present disclosure.

FIG. 13 is a flow chart for a method of determining camera-agnostic properties or features associated with points in one or more images, in accordance with embodiments of the present disclosure.

FIG. 14 illustrates candidate intersections of one or more candidate points with a projector ray, in accordance with embodiments of the present disclosure.

FIG. 15 is a flow chart for a method of determining camera-specific properties (also referred to as features) associated with points in one or more images, in accordance with embodiments of the present disclosure.

FIG. 16 illustrates information associated with a candidate intersection for input into a trained machine learning model, including a set of camera-specific properties or features and a set of camera-agnostic properties or features, in accordance with embodiments of the present disclosure.

FIG. 17 illustrates the candidate intersections of FIG. 14 with the addition of added probability information as output by a trained machine learning model, in accordance with embodiments of the present disclosure.

FIG. 18A is a flow chart for a method of adjusting probabilities for candidate intersections associated with a projector ray based on information about candidate intersections associated with one or more proximate projector rays, in accordance with embodiments of the present disclosure.

FIG. 18B illustrates an example of proximate projector rays, in accordance with embodiments of the present disclosure.

FIG. 19 is a flow chart for a method of adjusting probabilities for candidate intersections associated with a projector ray for a current frame based on information about the projector ray from one or more prior frames, in accordance with embodiments of the present disclosure.

FIG. 20 is a flow chart for a method of determining 3D coordinates for points in one or more 2D images using a trained machine learning model, in accordance with embodiments of the present disclosure.

FIG. 21 illustrates an example table of a plurality of points in one or more images, the table comprising, for each point of the plurality of points, one or more candidate intersections associated with the point, in accordance with embodiments of the present disclosure.

FIG. 22 illustrates an example table of a plurality of projector rays, the table comprising, for each projector ray of the plurality of projector rays, one or more candidate intersections associated with the projector ray, in accordance with embodiments of the present disclosure.

FIGS. 23A-C illustrate example input data sets for input into a trained machine learning model, in accordance with embodiments of the present disclosure.

FIG. 24 is a flow chart for a method of determining 3D coordinates for points in one or more 2D images, in accordance with embodiments of the present disclosure.

FIG. 25 is a flow chart for a method of determining candidate points for projector rays, in accordance with embodiments of the present disclosure.

FIG. 26 is a flow chart for a method of determining probabilities of candidate points being caused by projector rays, in accordance with embodiments of the present disclosure.

FIG. 27 is a flow chart for a method of selecting candidate points for projector rays, in accordance with embodiments of the present disclosure.

FIG. 28 is a flow chart for a method of determining 3D coordinates for points in one or more 2D images, where the points include a first set of points generated by light having a first wavelength and a second set of points generated by light having a second wavelength, in accordance with embodiments of the present disclosure.

FIG. 29A is a flow chart for a method of determining 3D coordinates for structured light features in one or more 2D images, in accordance with embodiments of the present disclosure.

FIG. 29B is a flow chart for a method of determining 3D coordinates for structured light features in one or more 2D images, in accordance with embodiments of the present disclosure.

FIG. 30A is a flow chart for a method of removing features from consideration for determination of 3D coordinates, in accordance with embodiments of the present disclosure.

FIG. 30B is a flow chart for a method of removing projector rays from consideration for determination of 3D coordinates, in accordance with embodiments of the present disclosure.

FIG. 30C is a flow chart for a method of removing projector rays from consideration for determination of 3D coordinates, in accordance with embodiments of the present disclosure.

FIG. 30D is a flow chart for a method of removing features from consideration for determination of 3D coordinates, in accordance with embodiments of the present disclosure.

FIG. 30E is a flow chart for a method of removing candidate intersections from consideration for determination of 3D coordinates, in accordance with embodiments of the present disclosure.

FIG. 31 is a flow chart for a method of determining 3D coordinates for structured light features in one or more 2D images that applies known ordering of structured light features and/or projector rays to eliminate candidates, in accordance with embodiments of the present disclosure.

FIG. 32 is a flow chart for a method of determining 3D coordinates for structured light features in one or more 2D images that eliminates structured light features from consideration that fail to satisfy one or more criteria, in accordance with embodiments of the present disclosure.

FIGS. 33A-D are schematic illustrations of a structured light projector projecting a structured light pattern and a camera capturing an image of the structured light pattern on an intraoral 3D surface, in accordance with some applications of the present disclosure.

FIG. 34A is a schematic illustration of a hexagonal grid structured light pattern, in accordance with some applications of the present disclosure.

FIG. 34B is a schematic illustration of a rectangular grid structured light pattern achieved by performing an affine transformation of the hexagonal grid structured pattern of FIG. 34A, in accordance with some applications of the present disclosure.

FIG. 34C is a schematic illustration of a rectangular grid structured light pattern in which an image is divided into quadrants about a solved point, in accordance with some applications of the present disclosure.

FIG. 35 illustrates a block diagram of an example computing device, in accordance with embodiments of the present disclosure.

### DETAILED DESCRIPTION

Described herein are methods and systems for processing images of dental surfaces illuminated by structured light and determining depth information for points on the dental surfaces based on captured points (e.g., structured light features) of the structured light pattern in the images. Reconstruction of a 3D point cloud from points in 2D images (e.g., spots in 2D images) requires solving a complex problem of correspondence matching between 2D points in the images and corresponding projector rays of structured light projected onto a dental surface. Embodiments disclosed herein apply machine learning techniques to assign probability scores to pairs of projector rays and candidate points (e.g., structured light features) that might have caused the projector rays, where such pairs may be referred to herein as candidate intersections or simply candidates. In embodiments, properties (also referred to as features) associated with candidate intersections are determined based on extraction of information from images and/or information about the cameras and/or structured light projectors associated with the candidate intersections. The extracted information may relate to matching quality, and may be processed by the trained machine learning model to assign, for each candidate intersection, a probability of a candidate point of the candidate intersection having been caused by the projector ray of the candidate intersection. In embodiments, a second trained machine learning model processes probabilities of candidate intersections being correct to select candidate intersections (e.g., to select, for each projector ray, one of the candidate points associated with that projector ray). Embodiments select candidate intersections in a manner that chooses “best” candidates for given projector rays while optimizing a global score summation. Accordingly, in embodiments a best candidate intersection may not be a highest probability candidate intersection for a given projector ray, but may instead be a candidate intersection that results in a highest combined probability of a set of candidate points corresponding to associated projector rays. As used herein, a candidate point for a projector ray is a point of structured light feature that might have been caused by the projector ray. A candidate point for a projector ray corresponds to a candidate intersection that includes the projector ray and the point/feature.

Embodiments provide a system and method for solving a correspondence problem between projector rays (e.g., points output by a structured light projector) and camera rays (e.g., points in images captured by cameras). In embodiments, the correspondence problem is solved with a maximal solve rate, a minimal error rate, and a minimal computation power usage. In embodiments, the correspondence problem is solved even in instances where some projector rays in the field of view of one or more cameras may be missed due, for example, to low signal quality. Additionally, in embodiments the correspondence problem is solved even in instances where some detected points are false points due to noise. In embodiments, the correspondence problem is solved in real time or near-real time as images are generated. Accordingly, as images are captured by an intraoral scanner, processing logic may solve the correspondence problem and determine 3D coordinates for points in the captured images to form point clouds. The point clouds may constitute intraoral scans, and may be stitched to previously captured 3D point clouds/intraoral scans and/or to a 3D surface generated from such previously captured 3D point clouds/intraoral scans.

Various embodiments are described herein. It should be understood that these various embodiments may be implemented as stand-alone solutions and/or may be combined. Accordingly, references to an embodiment, or one embodiment, may refer to the same embodiment and/or to different embodiments. Some embodiments are discussed herein with reference to intraoral scans and intraoral images. However, it should be understood that embodiments described with reference to intraoral scans also apply to lab scans or model/impression scans. A lab scan or model/impression scan may include one or more images of a dental site or of a model or impression of a dental site. Various embodiments are discussed with regards to candidate intersections. It should be understood that these embodiments also apply equally to candidate intersection groups, which are groups of candidate intersections from different images/cameras for which a same or similar intersection distance of a projector ray is determined.

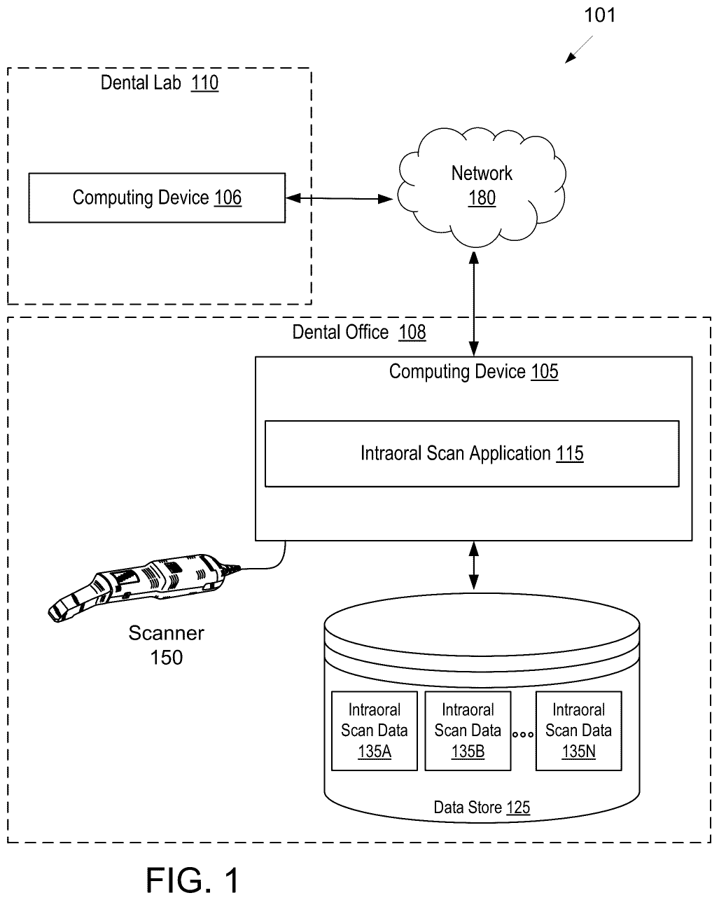

FIG. 1 illustrates one embodiment of a system 101 for performing intraoral scanning and/or generating a three-dimensional (3D) surface and/or a virtual three-dimensional model of a dental site. System 101 includes a dental office 108 and optionally one or more dental lab 110. The dental office 108 and the dental lab 110 each include a computing device 105, 106, where the computing devices 105, 106 may be connected to one another via a network 180. The network 180 may be a local area network (LAN), a public wide area network (WAN) (e.g., the Internet), a private WAN (e.g., an intranet), or a combination thereof.

Computing device 105 may be coupled to one or more intraoral scanner 150 (also referred to as a scanner) and/or a data store 125 via a wired or wireless connection. In one embodiment, multiple scanners 150 in dental office 108 wirelessly connect to computing device 105. In one embodiment, scanner 150 is wirelessly connected to computing device 105 via a direct wireless connection. In one embodiment, scanner 150 is wirelessly connected to computing device 105 via a wireless network. In one embodiment, the wireless network is a Wi-Fi network. In one embodiment, the wireless network is a Bluetooth network, a Zigbee network, or some other wireless network. In one embodiment, the wireless network is a wireless mesh network, examples of which include a Wi-Fi mesh network, a Zigbee mesh network, and so on. In an example, computing device 105 may be physically connected to one or more wireless access points and/or wireless routers (e.g., Wi-Fi access points/routers). Intraoral scanner 150 may include a wireless module such as a Wi-Fi module, and via the wireless module may join the wireless network via the wireless access point/router.

Computing device 106 may also be connected to a data store (not shown). The data stores may include local data stores and/or remote data stores. Computing device 105 and computing device 106 may each include one or more processing devices, memory, secondary storage, one or more input devices (e.g., such as a keyboard, mouse, tablet, touchscreen, microphone, camera, and so on), one or more output devices (e.g., a display, printer, touchscreen, speakers, etc.), and/or other hardware components.

Computing device 105 and/or data store 125 may be located at dental office 108 (as shown), at dental lab 110, or at one or more other locations such as a server farm that provides a cloud computing service. Computing device 105 and/or data store 125 may connect to components that are at a same or a different location from computing device 105 (e.g., components at a second location that is remote from the dental office 108, such as a server farm that provides a cloud computing service). For example, computing device 105 may be connected to a remote server, where some operations of intraoral scan application 115 are performed on computing device 105 and some operations of intraoral scan application 115 are performed on the remote server.

Intraoral scanner 150 may include a probe (e.g., a hand held probe) for optically capturing three-dimensional structures. The intraoral scanner 150 may be used to perform an intraoral scan of a patient's oral cavity. An intraoral scan application 115 running on computing device 105 may communicate with the scanner 150 to effectuate the intraoral scan. A result of the intraoral scan may be intraoral scan data 135A, 135B through 135N that may include one or more sets of intraoral scans and/or sets of intraoral 2D images. Each intraoral scan may include a 3D image or point cloud that may include depth information of a portion of a dental site. In embodiments, intraoral scans include x, y and z information.

A captured 3D image or point cloud may be generated based on multiple 2D images captured in parallel (e.g., at the same time) by different cameras. Scanner 150 may include one or more structured light projectors that output structured light at one or a few wavelengths, which may illuminate a dental site with the structured light. Multiple cameras of the scanner 150 may capture images of the dental site illuminated by the structured light from different angles. Captured images may be 2D images of the dental site illuminated by the structured light. Triangulation may be performed to determine the depth information about the 2D images. For example, each point of the structured light captured in one or more 2D images may have known 2D coordinates, but may initially lack depth information. The depth information may be determined using triangulation based on known information about a location of an origin of a projector ray of a structured light projector that caused a point to appear on the dental site and a location of a camera sensor that captured the point. Additionally, for points captured in multiple images (e.g., each image captured by a different camera), known locations of camera sensors of the two cameras may additionally or alternatively be used to perform triangulation and determine the depth of the point. However, it can be difficult to determine which projector rays correspond to which points (e.g., which spots or other structured light features) in the captured 2D images. Accordingly, in embodiments intraoral scan application 115 and/or other logic processes captured images to determine which points in captured images correspond to which projector rays of the structured light projector(s). Details for solving such a correspondence problem are set forth in greater detail below.

Intraoral scan data 135A-N may also include color 2D images and/or images of particular wavelengths (e.g., near-infrared (NIRI) images, infrared images, ultraviolet images, etc.) of a dental site in embodiments. In embodiments, intraoral scanner 150 alternates between generation of 3D intraoral scans (e.g., in which structured light is projected and 2D images of a dental site illuminated by the structured light are captured and processed to determine 3D point clouds) and one or more types of 2D intraoral images (e.g., color images, NIRI images, etc.) during scanning. For example, one or more 2D color images may be generated between generation of a fourth and fifth intraoral scan by outputting white light and capturing reflections of the white light using multiple cameras.

Intraoral scanner 150 may include multiple different cameras (e.g., each of which may include one or more image sensors) that generate additional 2D images (e.g., 2D color images) of different regions of a patient's dental arch concurrently. Intraoral 2D images may include 2D color images, 2D infrared or near-infrared (NIRI) images, and/or 2D images generated under other specific lighting conditions (e.g., 2D ultraviolet images). The 2D images may be used by a user of the intraoral scanner to determine where the scanning face of the intraoral scanner is directed and/or to determine other information about a dental site being scanned. The 2D images may also be used to apply a texture mapping to a 3D surface and/or 3D model of the dental site generated from the intraoral scans.

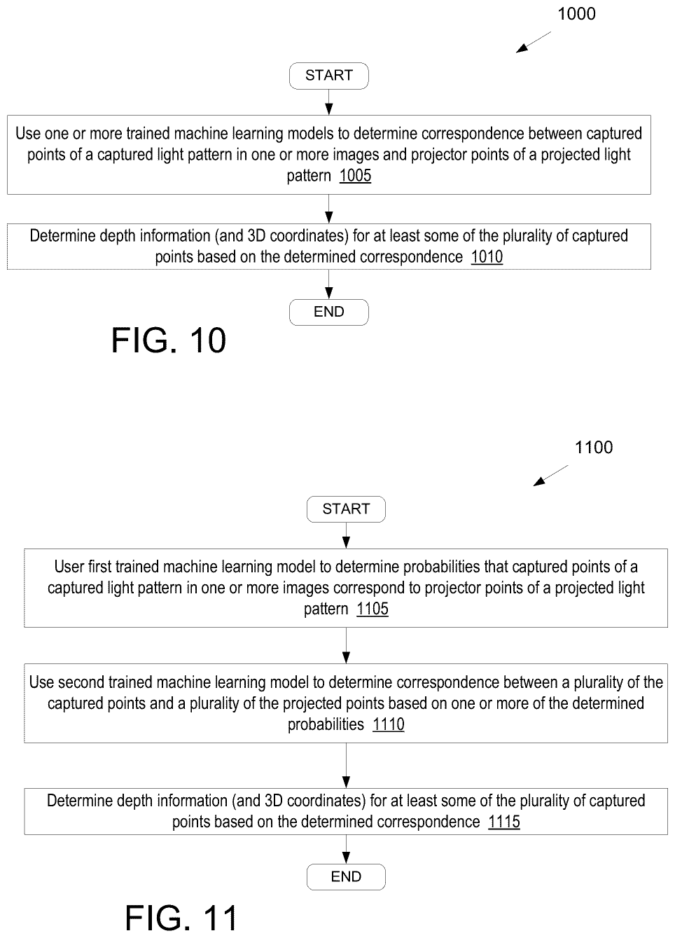

The scanner 150 may transmit the intraoral scan data 135A, 135B through 135N to the computing device 105. Computing device 105 may store some or all of the intraoral scan data 135A-135N in data store 125. In some embodiments, intraoral scan application 115 processes the intraoral scan data 135A-N to determine which points in captured 2D images correspond to which projector rays of structured light projectors, and to ultimately generate a 3D point cloud based on the points in the 2D images. The process of solving the correspondence problem and determining which points correspond to which projector rays is described in greater detail below with reference to FIGS. 10-34C.

According to an example, a user (e.g., a practitioner) may subject a patient to intraoral scanning. In doing so, the user may apply scanner 150 to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data 135A-N to computing device 105. The intraoral scan data 135A-N may be provided in the form of intraoral scan data sets, each of which may include 2D intraoral images (e.g., color 2D images) and/or 3D intraoral scans (e.g., based on 2D images of a dental site illuminated by structured light) of particular teeth and/or regions of an dental site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the scanner 150 to the computing device 105 in the form of one or more points (e.g., one or more pixels and/or groups of pixels). For instance, the scanner 150 may provide an intraoral scan as one or more point clouds. The intraoral scans may each comprise height information (e.g., a height map that indicates a depth for each pixel). In some embodiments, the intraoral scans include multiple 2D images of a dental site illuminated by one or more structured light projectors, which are then processed to generate a 3D point cloud. The processing of the 2D images may be performed on the scanner 150 before transmission to the computing device 105 or may be performed on the computing device 105 after receipt of the 2D images from scanner 150.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (dental site), or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a dental site within the oral cavity, or a real or virtual model thereof, or directed to the design and preparation of the dental site to receive such orthodontic elements. These elements may be appliances including but not limited to brackets and wires, retainers, clear aligners, or functional appliances.

In embodiments, intraoral scanning may be performed on a patient's oral cavity during a visitation of dental office 108. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The dental information (intraoral scan data 135A-N) generated from the intraoral scanning may include 3D scan data, 2D color images, NIRI and/or infrared images, and/or ultraviolet images, of all or a portion of the upper jaw and/or lower jaw. The intraoral scan data 135A-N may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the scanner 150 inside a patient's mouth to capture all viewpoints of one or more tooth. During scanning, the scanner 150 is calculating distances to solid surfaces in some embodiments. These distances may be recorded as 3D point clouds in some embodiments. Each scan (e.g., point cloud) is overlapped algorithmically, or ‘stitched’, with the previous set of scans to generate a growing 3D surface. As such, each scan is associated with a rotation in space, or a projection, to how it fits into the 3D surface.

During intraoral scanning, intraoral scan application 115 may register and stitch together two or more intraoral scans generated thus far from the intraoral scan session to generate a growing 3D surface. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. A view of the 3D surface(s) may be periodically or continuously updated according to one or more viewing modes of the intraoral scan application. In one viewing mode, the 3D surface may be continuously updated such that an orientation of the 3D surface that is displayed aligns with a field of view of the intraoral scanner (e.g., so that a portion of the 3D surface that is based on a most recently generated intraoral scan is approximately centered on the display or on a window of the display) and a user sees what the intraoral scanner sees. In one viewing mode, a position and orientation of the 3D surface is static, and an image of the intraoral scanner is optionally shown to move relative to the stationary 3D surface.

Intraoral scan application 115 may generate one or more 3D surfaces from intraoral scans, and may display the 3D surfaces to a user (e.g., a doctor) via a graphical user interface (GUI) during intraoral scanning. In embodiments, separate 3D surfaces are generated for the upper jaw and the lower jaw. This process may be performed in real time or near-real time to provide an updated view of the captured 3D surfaces during the intraoral scanning process. As scans are received, these scans may be registered and stitched to a 3D surface.

When a scan session or a portion of a scan session associated with a particular scanning role (e.g., upper jaw role, lower jaw role, bite role, etc.) is complete (e.g., all scans for an dental site or dental site have been captured), intraoral scan application 115 may generate a virtual 3D model of one or more scanned dental sites (e.g., of an upper jaw and a lower jaw). The final 3D model may be a set of 3D points and their connections with each other (i.e. a mesh). To generate the virtual 3D model, intraoral scan application 115 may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 115 may match points of one scan with the closest points interpolated on the surface of another scan, and iteratively minimize the distance between matched points. Other registration techniques may also be used.

Intraoral scan application 115 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 115 may integrate intraoral scans into a single virtual 3D model by applying the appropriate determined transformations to each of the intraoral scans. Each transformation may include rotations about one to three axes and translations within one to three planes.

Intraoral scan application 115 may generate one or more 3D models from intraoral scans, and may display the 3D models to a user (e.g., a doctor) via a graphical user interface (GUI). The 3D models can then be checked visually by the doctor. The doctor can virtually manipulate the 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction. If scaling of image on screen is also considered, than the doctor can virtually manipulate the 3D models with respect to up to seven degrees of freedom (the previously described six degrees of freedom in addition to zoom or scale).

After completion of the 3D surface(s) and/or 3D model(s) and/or during generation of the 3D surface(s) and/or 3D model(s) intraoral scan application may perform texture mapping to map color information to the 3D surface(s) and/or 3D model(s). Color images (e.g., images generated under white light conditions) may be processed, and color information from these color images may be added to the 3D surface(s) and/or 3D model(s). In some embodiments, the color information is used to improve an accuracy of solving the correspondence problem for at least some points in captured 2D images of intraoral scans.

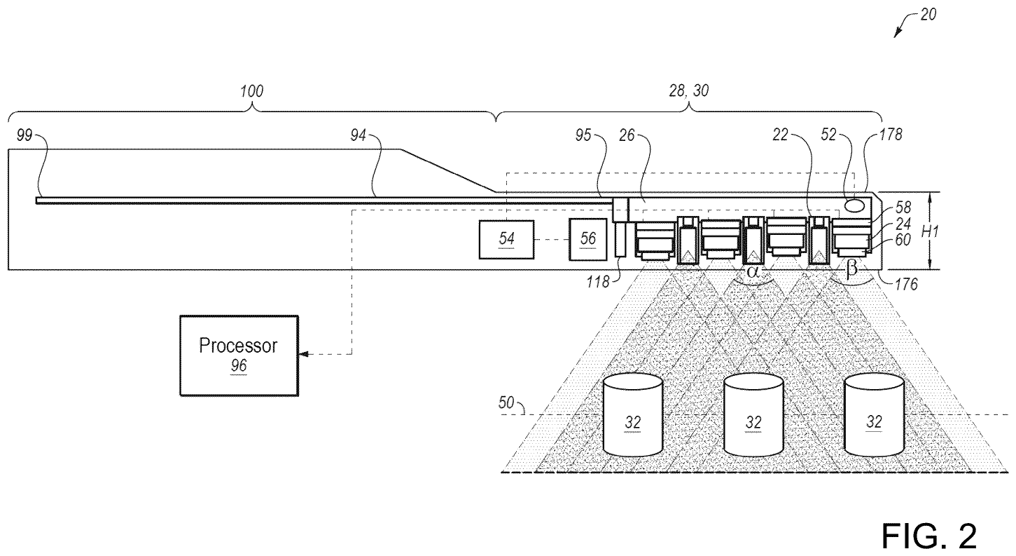

Reference is now made to FIG. 2, which is a schematic illustration of an intraoral scanner 20 comprising an elongate wand (e.g., an elongate handheld wand), in accordance with some applications of the present disclosure. The intraoral scanner 20 may correspond to intraoral scanner 150 of FIG. 1 in embodiments. Intraoral scanner 20 includes a plurality of structured light projectors 22 and a plurality of cameras 24 that are coupled to a rigid structure 26 disposed within a probe 28 at a distal end 30 of the intraoral scanner 20. In some applications, during an intraoral scanning procedure, probe 28 is inserted into the oral cavity of a subject or patient.

For some applications, structured light projectors 22 are positioned within probe 28 such that each structured light projector 22 faces an object 32 outside of intraoral scanner 20 that is placed in its field of illumination, as opposed to positioning the structured light projectors in a proximal end of the handheld wand and illuminating the object by reflection of light off a mirror and subsequently onto the object. In embodiments, the structured light projectors 22 and cameras 24 are a distance of less than 20 mm from the object 32, or less than 15 mm from the object 32, or less than 10 mm from the object 32. The distance may be measured as a distance between a camera/structured light projector and a plane orthogonal to an imaging axis of the intraoral scanner (e.g., where the imaging axis of the intraoral scanner may be perpendicular to a longitudinal axis of the intraoral scanner). Alternatively, the distance may be measured differently for each camera as a distance from the camera to the object 32 along a ray from the camera to the object.

In some embodiments, the structured light projectors are disposed at a distal end of the wand and face an object to be scanned (e.g., is directed approximately orthogonal to a longitudinal axis of a probe of the wand). In some embodiments, one or more structured light projectors are oriented approximately parallel to the longitudinal axis of the probe and face a mirror in the probe, which redirects structured light projected by the structured light projector(s) onto an object to be imaged. Similarly, for some applications, cameras 24 are positioned within probe 28 at a distal end of the probe such that each camera 24 faces an object 32 outside of intraoral scanner 20 that is placed in its field of view. In some embodiments, one or more cameras is oriented approximately parallel to the longitudinal axis of the probe and toward a mirror, and views the object by reflection of light off the mirror and into the camera. In some embodiments, the projectors and cameras of the intraoral scanner are arranged into component groups (e.g., imaging units) each including one or more structured light projectors and one or more cameras. In embodiments, component groups are arranged in series along the longitudinal axis of the probe. In an embodiment, each component group includes a projector with two or more cameras disposed about the projector. By combining multiple cameras and structured light projectors within probe 28, the scanner is able to have an overall large field of view while maintaining a low profile probe.

In some applications, cameras 24 each have a large field of view β (beta) of at least 45 degrees, e.g., at least 70 degrees, e.g., at least 80 degrees, e.g., 85 degrees. In some applications, the field of view may be less than 120 degrees, e.g., less than 100 degrees, e.g., less than 90 degrees. In one embodiment, a field of view β (beta) for each camera is between 80 and 90 degrees, which may be particularly useful because it provided a good balance among pixel size, field of view and camera overlap, optical quality, and cost. Cameras 24 may include an image sensor 58 and objective optics 60 including one or more lenses. To enable close focus imaging, cameras 24 may focus at an object focal plane 50 that is located between 1 mm and 30 mm, e.g., between 4 mm and 24 mm, e.g., between 5 mm and 11 mm, e.g., 9 mm-10 mm, from the lens that is farthest from the sensor. In some applications, cameras 24 may capture images at a frame rate of at least 30 frames per second, e.g., at a frame of at least 75 frames per second, e.g., at least 100 frames per second. In some applications, the frame rate may be less than 200 frames per second.

A large field of view achieved by combining the respective fields of view of all the cameras (e.g., of multiple component groups) may improve accuracy due to reduced amount of image stitching errors, especially in edentulous regions, where the gum surface is smooth and there may be fewer clear high resolution 3D features. Having a larger field of view enables large smooth features, such as the overall curve of the tooth, to appear in each image frame, which improves the accuracy of stitching respective surfaces obtained from multiple such image frames.

Similarly, structured light projectors 22 may each have a large field of illumination α (alpha) of at least 45 degrees, e.g., at least 70 degrees. In some applications, field of illumination α (alpha) may be less than 120 degrees, e.g., than 100 degrees.

For some applications, in order to improve image capture, each camera 24 has a plurality of discrete preset focus positions, in each focus position the camera focusing at a respective object focal plane 50. Each of cameras 24 may include an autofocus actuator that selects a focus position from the discrete preset focus positions in order to improve a given image capture. Additionally or alternatively, each camera 24 includes an optical aperture phase mask that extends a depth of focus of the camera, such that images formed by each camera are maintained focused over all object distances located between 1 mm and 30 mm, e.g., between 4 mm and 24 mm, e.g., between 5 mm and 11 mm, e.g., 9 mm-10 mm, from the lens that is farthest from the sensor.

In some applications, structured light projectors 22 and cameras 24 are coupled to rigid structure 26 in a closely packed and/or alternating fashion, such that (a) a substantial part of each camera's field of view overlaps the field of view of neighboring cameras, and (b) a substantial part of each camera's field of view overlaps the field of illumination of neighboring projectors. Optionally, at least 20%, e.g., at least 50%, e.g., at least 75% of the projected pattern of light are in the field of view of at least one of the cameras at an object focal plane 50 that is located at least 4 mm from the lens that is farthest from the sensor. Due to different possible configurations of the projectors and cameras, some of the projected pattern may never be seen in the field of view of any of the cameras, and some of the projected pattern may be blocked from view by object 32 as the scanner is moved around during a scan.

Rigid structure 26 may be a non-flexible structure to which structured light projectors 22 and cameras 24 are coupled so as to provide structural stability to the optics within probe 28. Coupling all the projectors and all the cameras to a common rigid structure helps maintain geometric integrity of the optics of each structured light projector 22 and each camera 24 under varying ambient conditions, e.g., under mechanical stress as may be induced by the subject's mouth. Additionally, rigid structure 26 helps maintain stable structural integrity and positioning of structured light projectors 22 and cameras 24 with respect to each other.

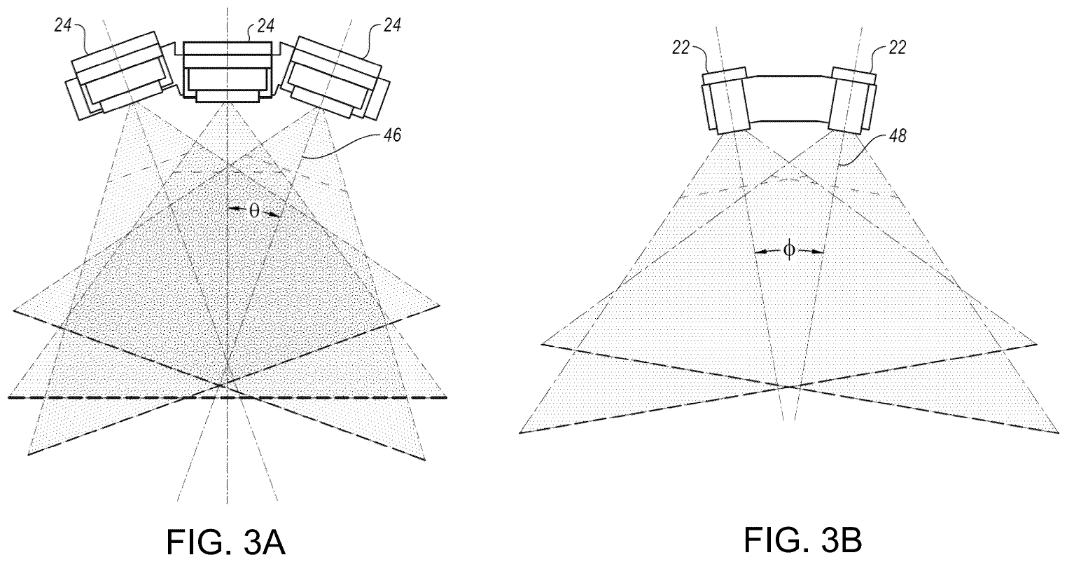

Reference is now made to FIGS. 3A-3B, which include schematic illustrations of a positioning configuration for cameras 24 and structured light projectors 22 respectively, in accordance with some applications of the present disclosure. For some applications, in order to improve the overall field of view and field of illumination of the intraoral scanner 20, cameras 24 and structured light projectors 22 are positioned such that they do not all face the same direction. For some applications, such as is shown in FIG. 3A, a plurality of cameras 24 are coupled to rigid structure 26 such that an angle θ (theta) between two respective optical axes 46 of at least two cameras 24 is 90 degrees or less, e.g., 35 degrees or less. Similarly, for some applications, such as is shown in FIG. 3B, a plurality of structured light projectors 22 are coupled to rigid structure 26 such that an angle q (phi) between two respective optical axes 48 of at least two structured light projectors 22 is 90 degrees or less, e.g., 35 degrees or less.

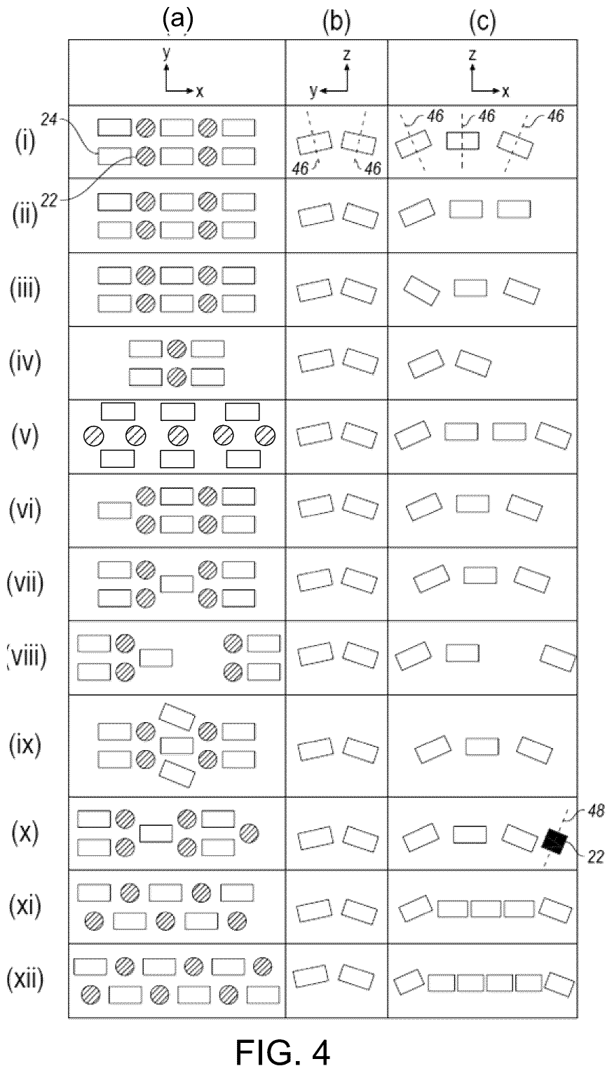

Reference is now made to FIG. 4, which is a chart depicting a plurality of different configurations for the position of structured light projectors 22 and cameras 24 in probe 28, in accordance with some applications of the present disclosure. Structured light projectors 22 are represented in FIG. 4 by circles and cameras 24 are represented in FIG. 4 by rectangles. It is noted that rectangles are used to represent the cameras, since typically, each image sensor 58 and the field of view β (beta) of each camera 24 have aspect ratios of 1:2. Column (a) of FIG. 4 shows a bird's eye view of the various configurations of structured light projectors 22 and cameras 24. The x-axis as labeled in the first row of column (a) corresponds to a central longitudinal axis of probe 28. Column (b) shows a side view of cameras 24 from the various configurations as viewed from a line of sight that is coaxial with the central longitudinal axis of probe 28 and substantially parallel to a viewing axis of the intraoral scanner. Similarly to as shown in FIG. 3A, column (b) of FIG. 4 shows cameras 24 positioned so as to have optical axes 46 at an angle of 90 degrees or less, e.g., 35 degrees or less, with respect to each other. Column (c) shows a side view of cameras 24 of the various configurations as viewed from a line of sight that is perpendicular to the central longitudinal axis of probe 28.

Typically, the distal-most (toward the positive x-direction in FIG. 4) and proximal-most (toward the negative x-direction in FIG. 4) cameras 24 are positioned such that their optical axes 46 are slightly turned inwards, e.g., at an angle of 90 degrees or less, e.g., 35 degrees or less, with respect to the next closest camera 24. The camera(s) 24 that are more centrally positioned, i.e., not the distal-most camera 24 nor proximal-most camera 24, are positioned so as to face directly out of the probe, their optical axes 46 being substantially perpendicular to the central longitudinal axis of probe 28. It is noted that in row (xi) a projector 22 is positioned in the distal-most position of probe 28, and as such the optical axis 48 of that projector 22 points inwards, allowing a larger number of spots 33 projected from that particular projector 22 to be seen by more cameras 24.

In embodiments, the number of structured light projectors 22 in probe 28 may range from two, e.g., as shown in row (iv) of FIG. 4, to six, e.g., as shown in row (xii). Typically, the number of cameras 24 in probe 28 may range from four, e.g., as shown in row (iv), to seven, e.g., as shown in row (ix). It is noted that the various configurations shown in FIG. 4 are by way of example and not limitation, and that the scope of the present disclosure includes additional configurations not shown. For example, the scope of the present disclosure includes fewer or more than five projectors 22 positioned in probe 28 and fewer or more than seven cameras positioned in probe 28. With reference to row (v), two outer rows include a series of cameras and an inner row includes a series of projectors.

In an example application, an apparatus for intraoral scanning (e.g., an intraoral scanner 150) includes an elongate wand comprising a probe at a distal end of the elongate handheld wand, at least two light projectors disposed within the probe, and at least four cameras disposed within the probe. Each light projector may include at least one light source configured to generate light when activated, and a pattern generating optical element that is configured to generate a pattern of light when the light is transmitted through the pattern generating optical element. Each of the at least four cameras may include a camera sensor (also referred to as an image sensor) and one or more lenses, wherein each of the at least four cameras is configured to capture a plurality of images that depict at least a portion of the projected pattern of light on an intraoral surface. A majority of the at least two light projectors and the at least four cameras may be arranged in at least two rows that are each approximately parallel to a longitudinal axis of the probe, the at least two rows comprising at least a first row and a second row. In at least one embodiment, as shown in row (v), the intraoral scanner includes two rows of cameras (e.g., two rows of three cameras each) and a single row of structured light projectors (e.g., five structured light projectors) disposed between the two rows of cameras.

In a further application, a distal-most camera along the longitudinal axis and a proximal-most camera along the longitudinal axis of the at least four cameras are positioned such that their optical axes are at an angle of 90 degrees or less with respect to each other from a line of sight that is perpendicular to the longitudinal axis. Cameras in the first row and cameras in the second row may and/or third row be positioned such that optical axes of the cameras in the first row are at an angle of 90 degrees or less with respect to optical axes of the cameras in the second row and/or third row from a line of sight that is coaxial with the longitudinal axis of the probe. A remainder of the at least four cameras other than the distal-most camera and the proximal-most camera have optical axes that are substantially parallel to the longitudinal axis of the probe. Some of the at least two rows may include an alternating sequence of light projectors and cameras. In some embodiments, some rows contain only projectors and some rows contain only cameras (e.g., as shown in row (v).

In a further application, the distal-most camera along the longitudinal axis and the proximal-most camera along the longitudinal axis are positioned such that their optical axes are at an angle of 35 degrees or less with respect to each other from the line of sight that is perpendicular to the longitudinal axis. The cameras in the first row and the cameras in the second row and/or third row may be positioned such that the optical axes of the cameras in the first row are at an angle of 35 degrees or less with respect to the optical axes of the cameras in the second row and/or third row from the line of sight that is coaxial with the longitudinal axis of the probe.

In a further application, the at least four cameras may have a combined field of view of 25-45 mm along the longitudinal axis and a field of view of 20-40 mm along a z-axis corresponding to distance from the probe.

Returning to FIG. 2, for some applications, there is at least one uniform light projector 118 (which may be an unstructured light projector that projects light across a range of wavelengths) coupled to rigid structure 26. Uniform light projector 118 may transmit white light onto object 32 being scanned. At least one camera, e.g., one of cameras 24, captures two-dimensional color images of object 32 using illumination from uniform light projector 118.

Processor 96 may run a surface reconstruction algorithm that may use detected patterns (e.g., dot patterns) projected onto object 32 to generate a 3D surface of the object 32, as described in greater detail below with reference to FIGS. 10-34C. In some embodiments, the processor 96 may combine at least one 3D scan captured using illumination from structured light projectors 22 with a plurality of intraoral 2D images captured using illumination from uniform light projector 118 in order to generate a digital three-dimensional image of the intraoral three-dimensional surface. Using a combination of structured light and uniform illumination enhances the overall capture of the intraoral scanner and may help reduce the number of options that processor 96 needs to consider when running a correspondence algorithm used to detect depth values for object 32. In one embodiment, the intraoral scanner and correspondence algorithm described in U.S. Application Ser. No. 16/446,181, filed Jun. 19, 2019, is used. U.S. Application Ser. No. 16/446,181, filed Jun. 19, 2019, is incorporated by reference herein in its entirety. In embodiments, processor 92 may be a processor of computing device 105 of FIG. 1. Alternatively, processor 92 may be a processor integrated into the intraoral scanner 20.

For some applications, all data points taken at a specific time are used as a rigid point cloud, and multiple such point clouds are captured at a frame rate of over 10 captures per second. The plurality of point clouds are then stitched together using a registration algorithm, e.g., iterative closest point (ICP), to create a dense point cloud. A surface reconstruction algorithm may then be used to generate a representation of the surface of object 32.

For some applications, at least one temperature sensor 52 is coupled to rigid structure 26 and measures a temperature of rigid structure 26. Temperature control circuitry 54 disposed within intraoral scanner 20 (a) receives data from temperature sensor 52 indicative of the temperature of rigid structure 26 and (b) activates a temperature control unit 56 in response to the received data. Temperature control unit 56, e.g., a PID controller, keeps probe 28 at a desired temperature (e.g., between 35 and 43 degrees Celsius, between 37 and 41 degrees Celsius, etc.). Keeping probe 28 above 35 degrees Celsius, e.g., above 37 degrees Celsius, reduces fogging of the glass surface of intraoral scanner 20, through which structured light projectors 22 project and cameras 24 view, as probe 28 enters the intraoral cavity, which is typically around or above 37 degrees Celsius. Keeping probe 28 below 43 degrees, e.g., below 41 degrees Celsius, prevents discomfort or pain.

In some embodiments, heat may be drawn out of the probe 28 via a heat conducting element 94, e.g., a heat pipe, that is disposed within intraoral scanner 20, such that a distal end 95 of heat conducting element 94 is in contact with rigid structure 26 and a proximal end 99 is in contact with a proximal end 100 of intraoral scanner 20. Heat is thereby transferred from rigid structure 26 to proximal end 100 of intraoral scanner 20. Alternatively or additionally, a fan disposed in a handle region 174 of intraoral scanner 20 may be used to draw heat out of probe 28.

FIGS. 2-4 illustrate one type of intraoral scanner that can be used for embodiments of the present disclosure. However, it should be understood that embodiments are not limited to the illustrated type of intraoral scanner. In one embodiment, intraoral scanner 150 corresponds to the intraoral scanner described in U.S. application Ser. No. 16/910,042, filed Jun. 23, 2020 and entitled “Intraoral 3D Scanner Employing Multiple Miniature Cameras and Multiple Miniature Pattern Projectors”, which is incorporated by reference herein. In one embodiment, intraoral scanner 150 corresponds to the intraoral scanner described in U.S. Application Ser. No. 16/446,181, filed Jun. 19, 2019 and entitled “Intraoral 3D Scanner Employing Multiple Miniature Cameras and Multiple Miniature Pattern Projectors”, which is incorporated by reference herein.

In embodiments, the scanner 150 of FIG. 1 generates a light pattern comprising a distribution of discrete unconnected spots of light. Alternatively, other light patterns may be generated, such as a pattern of discrete unconnected lines, a pattern of intersecting lines, a checkerboard pattern, a pattern of other shapes, etc. In embodiments, the pattern of light is not time varying. Alternatively, the pattern of light may be time varying. In some embodiments, scanner 150 includes multiple structured light projectors, where some of the structured light projectors output structured light having a first wavelength and other structured light projectors output structured light having a second wavelength. In other embodiments, scanner 150 may include structured light projectors that project more than two wavelengths (e.g., one or more first structured light projectors that project light having a first wavelength, one or more second structured light projectors that project light having a second wavelength, one or more third structured light projectors that project light having a third wavelength, and so on.

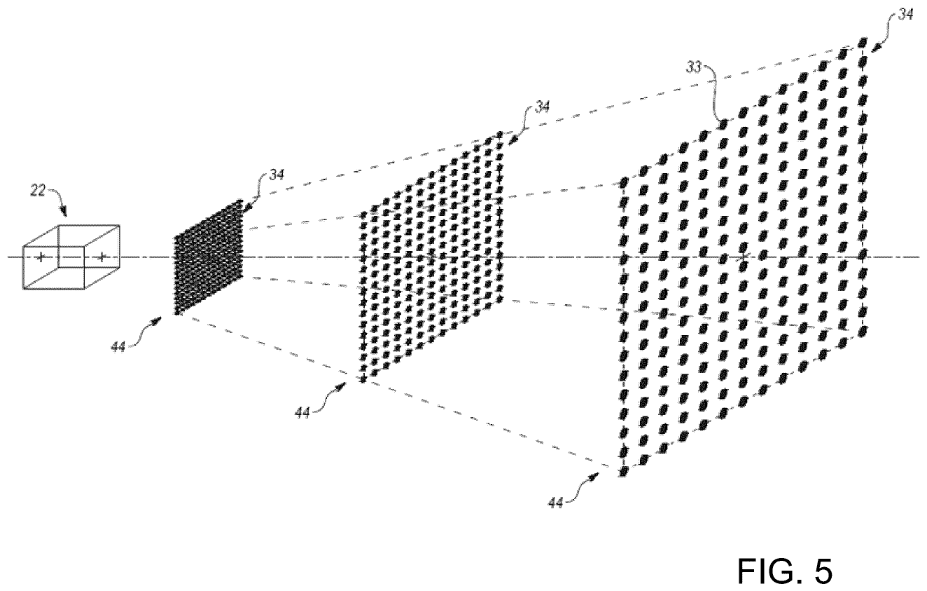

Reference is now made to FIG. 5, which is a schematic illustration of a structured light projector 22 projecting a distribution of discrete unconnected spots of light onto a plurality of object focal planes, in accordance with some applications of the present invention. Object 32 of FIG. 2 being scanned may be one or more teeth or other intraoral object/tissue inside a subject's mouth. The somewhat translucent and glossy properties of teeth may affect the contrast of the structured light pattern being projected. For example, (a) some of the light hitting the teeth may scatter to other regions within the intraoral scene, causing an amount of stray light, and (b) some of the light may penetrate the tooth and subsequently come out of the tooth at any other point. Thus, in order to improve image capture of an intraoral scene under structured light illumination, without using contrast enhancement means such as coating the teeth with an opaque powder, a sparse distribution 34 of discrete unconnected spots of light may be used to provide an improved balance between reducing the amount of projected light while maintaining a useful amount of information. In one embodiment, the sparseness of distribution 34 may be characterized by a ratio of: (a) illuminated area on an orthogonal plane 44 in field of illumination α (alpha), i.e., the sum of the area of all projected spots 33 on the orthogonal plane 44 in field of illumination α (alpha), to (b) non-illuminated area on orthogonal plane 44 in field of illumination α (alpha). In some applications, sparseness ratio may be at least 1:150 and/or less than 1:16 (e.g., at least 1:64 and/or less than 1:36).

In some applications, each structured light projector 22 projects at least 400 (e.g., 550) discrete unconnected spots 33 onto an intraoral three-dimensional surface during a scan. In embodiments, each structured light projector 22 projects a plurality of projector rays or beams, each corresponding to a discrete spot. In some applications, each structured light projector 22 projects less than 3000 discrete unconnected spots 33 onto an intraoral surface during a scan. In order to reconstruct the three-dimensional surface from projected sparse distribution 34, correspondence between respective projected spots 33 and the spots detected by cameras 24 should be determined, as further described hereinbelow with reference to FIGS. 10-34C.

For some applications, structured light projector 22 includes a light source (e.g., a laser diode) and a pattern generating optical element. The pattern generating optical element may be, for example, a diffractive optical element (DOE) that generates distribution 34 of discrete unconnected spots 33 of light when the light source transmits light through DOE onto object 32. As used herein, a spot of light is defined as a small area of light having any shape. For some applications, different structured light projectors 22 generate spots having different respective shapes, i.e., every spot 33 generated by a specific DOE has the same shape, and the shape of spots 33 generated by at least one DOE is different from the shape of spots generated by at least one other DOE 39. Alternatively, different structured light projectors project spots having the same shape. In some embodiments, different light projectors project spots having a different wavelength or color (e.g., structured light projectors may be divided into a first class that emits a first wavelength, a second class that emits a second wavelength, a third class that emits a third wavelength, and so on). By way of example, some pattern generating optical elements may generate circular spots 33 (such as is shown in FIG. 5), some of pattern generating optical elements may generate square spots, and some of the pattern generating optical elements may generate elliptical spots. Optionally, some pattern generating optical elements may generate line patterns, connected or unconnected spots, checkerboard patterns, etc.

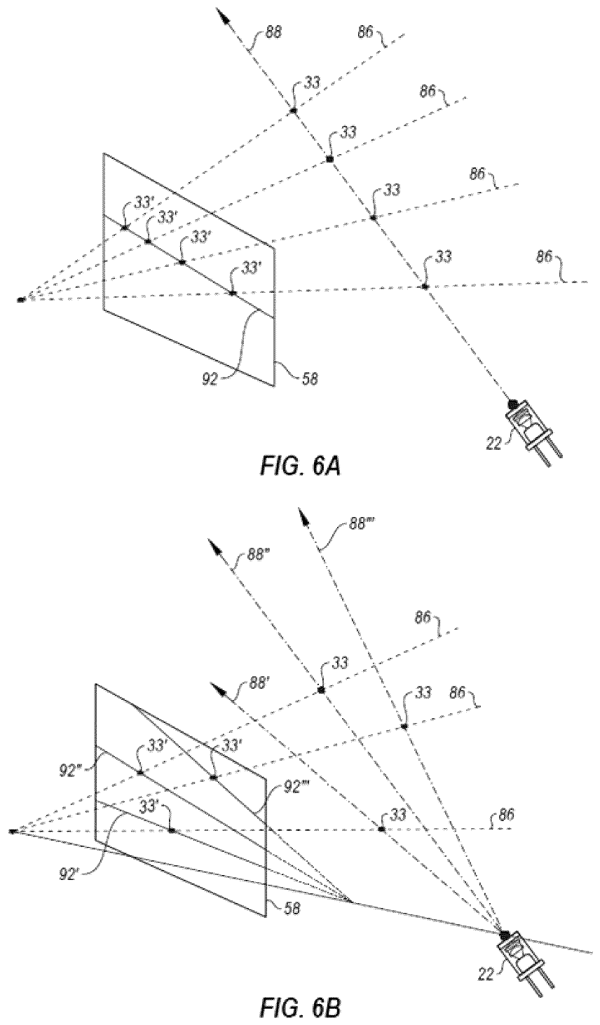

Reference is now made to FIGS. 6A-B, which are schematic illustrations of a structured light projector 22 projecting discrete unconnected spots 33 and a camera sensor 58 detecting spots 33′, in accordance with some applications of the present disclosure. For some applications, a method is provided for determining correspondence between the projected spots 33 or other points on the intraoral surface and detected spots 33′ or other points on respective camera sensors 58. Once the correspondence is determined, a three-dimensional image of the surface is reconstructed. Each camera sensor 58 has an array of pixels, for each of which there exists a corresponding camera ray 86 and/or point an image captured by camera sensor 58. Similarly, for each projected spot 33 and/or other point from each projector 22 there exists a corresponding projector ray 88. Each projector ray 88 corresponds to a respective path 92 of pixels on at least one of camera sensors 58. Thus, if a camera sees a spot 33′ or other point projected by a specific projector ray 88, that spot 33′ or other point will necessarily be detected by a pixel on the specific path 92 of pixels that corresponds to that specific projector ray 88.

With specific reference to FIG. 6B, the correspondence between respective projector rays 88 and respective camera sensor paths 92 is shown. Projector ray 88′ corresponds to camera sensor path 92′, projector ray 88″ corresponds to camera sensor path 92″, and projector ray 88′″ corresponds to camera sensor path 92″. For example, if a specific projector ray 88 were to project a spot into a dust-filled space, a line of dust in the air would be illuminated. The line of dust as detected by camera sensor 58 would follow the same path on camera sensor 58 as the camera sensor path 92 that corresponds to the specific projector ray 88.

In embodiments, during a calibration process one or more machine learning models are trained using image data captured from one or more known objects (e.g., one or more calibration objects). Training the machine learning models requires huge amount of data that is fed to the machine learning models. In one embodiment, a reference object is first scanned with an intraoral scanner. The reference model may be a model for which processing logic has a ground-truth surface mesh (e.g., generated using an external, high resolution/accurate scanner. Resulting data, including all candidate rays, may be input into a dedicated file or data store. In one embodiment, using a “stitch-to-reference” technique processing logic takes the trajectories of the intraoral scanner (determined by the stitching of the captured images to the reference) and simulates spots using calibrated projectors' rays, and shooting them onto the reference surfaces. This provides simulated spots and simulated ray distances. These simulated spots and ray distances can then be compared to “real” candidates to determine if a candidate that is close enough to be considered as the correct candidate is identified. In embodiments, candidates that did not fulfill this rule will be considered as wrong candidates.

Additionally, or alternatively, calibration values may be stored based on camera rays 86 corresponding to pixels on camera sensor 58 of each one of cameras 24, and projector rays 88 corresponding to projected spots 33 of light from each structured light projector 22. For example, calibration values may be stored for (a) a plurality of camera rays 86 corresponding to a respective plurality of pixels on camera sensor 58 of each one of cameras 24, and (b) a plurality of projector rays 88 corresponding to a respective plurality of projected spots 33 of light from each structured light projector 22.

By way of example, the following calibration process may be used. A high accuracy dot target, e.g., black dots on a white background, is illuminated from below and an image is taken of the target with all the cameras. The dot target is then moved perpendicularly toward the cameras, i.e., along the z-axis, to a target plane. The dot-centers are calculated for all the dots in all respective z-axis positions to create a three-dimensional grid of dots in space. A distortion and camera pinhole model is then used to find the pixel coordinate for each three-dimensional position of a respective dot-center, and thus a camera ray is defined for each pixel as a ray originating from the pixel whose direction is towards a corresponding dot-center in the three-dimensional grid. The camera rays corresponding to pixels in between the grid points can be interpolated. The above-described camera calibration procedure is repeated for all respective wavelengths of respective laser diodes 36, such that included in the stored calibration values are camera rays 86 corresponding to each pixel on each camera sensor 58 for each of the wavelengths.

After cameras 24 have been calibrated and all camera ray 86 values stored, structured light projectors 22 may be calibrated as follows. A flat featureless target is used and structured light projectors 22 are turned on one at a time. Each spot is located on at least one camera sensor 58. Since cameras 24 are now calibrated, the three-dimensional spot location of each spot is computed by triangulation based on images of the spot in multiple different cameras. The above-described process is repeated with the featureless target located at multiple different z-axis positions. Each projected spot on the featureless target will define a projector ray in space originating from the projector.

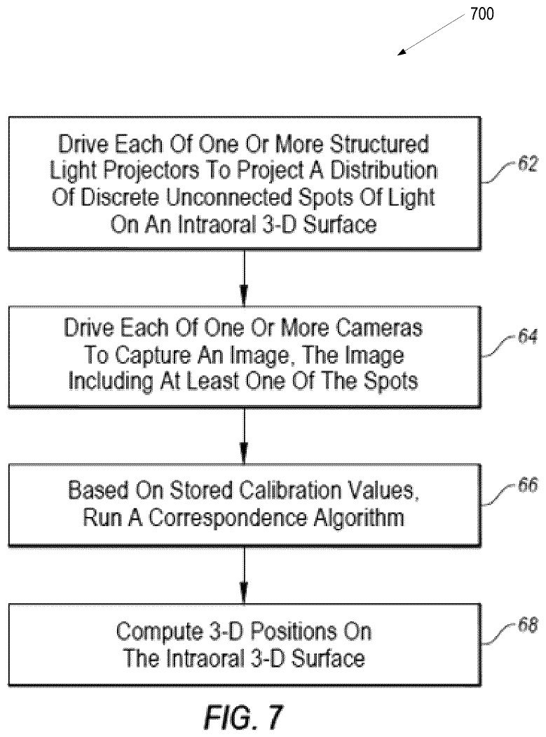

Reference is now made to FIG. 7, which is a flow chart outlining a method 700 for generating a digital three-dimensional image, in accordance with some applications of the present invention. At blocks 62 and 64, respectively, of method 700 each structured light projector 22 is driven to project distribution 34 of discrete unconnected spots 33 of light on an intraoral three-dimensional surface, and each camera 24 is driven to capture an image that includes at least one of spots 33. Based on the stored calibration values indicating (a) a camera ray 86 corresponding to each pixel on camera sensor 58 of each camera 24, and (b) a projector ray 88 corresponding to each projected spot 33 of light from each structured light projector 22, one or more possibilities for matching points on the captured image(s) to projector rays are determined. Each possibility may be a candidate intersection. Information on the candidate intersections may be processed using a first trained machine learning model, which may output, for each candidate intersection, a probability of a point (e.g., structured light feature, or portion thereof) having been generated by a projector ray. In embodiments, information on the probabilities determined for the candidate intersections are input into a second trained machine learning model, which outputs selections of one or more candidate intersections. The processing of the candidate intersections using the one or more trained machine learning models may be referred to as running a correspondence algorithm in embodiments, where the correspondence algorithm may be machine learning model-based in embodiments. Once the correspondence is solved, 3D positions on the intraoral surface are computed at block 68 and used to generate a digital 3D surface and/or model of the intraoral surface.

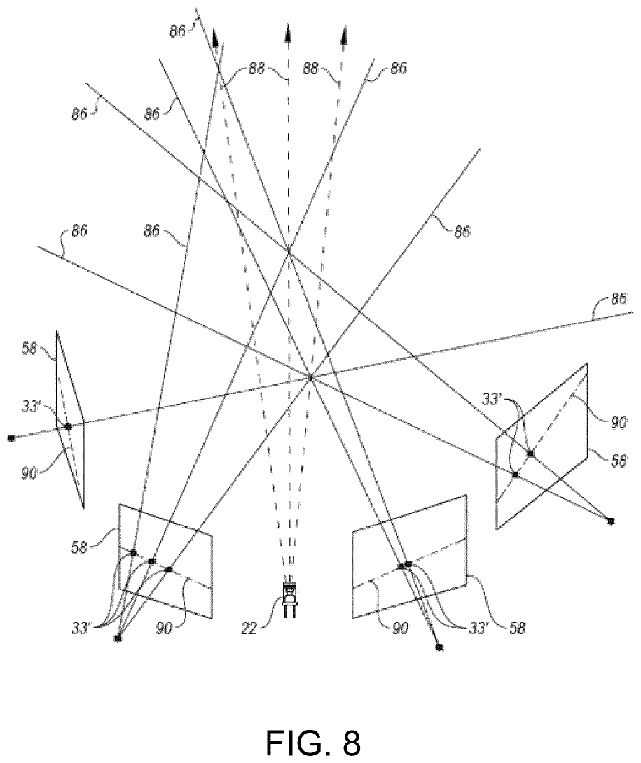

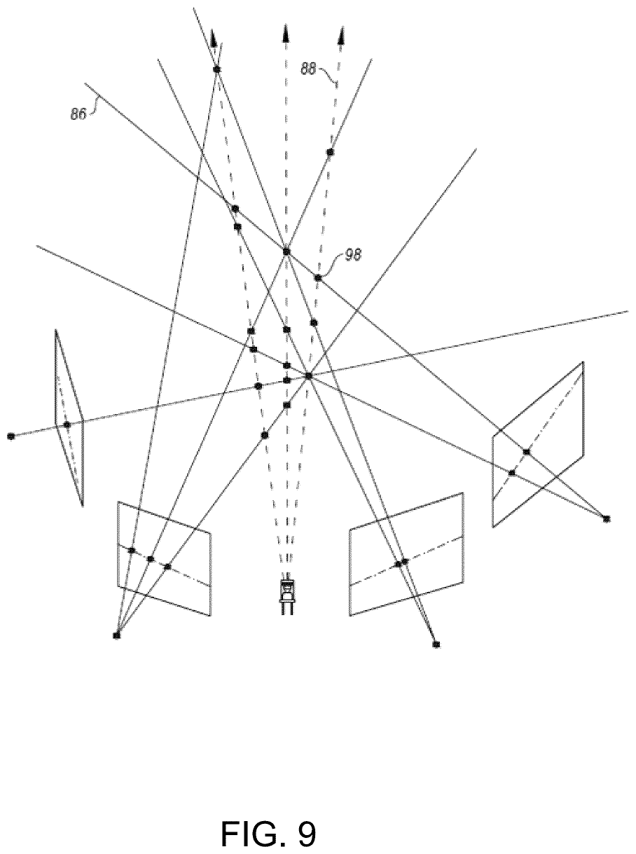

Reference is now made to FIGS. 8-9, which are schematic illustrations of a structured light projector 22 projecting discrete unconnected spots and multiple camera sensors 58 detecting spots, in accordance with some applications of the present disclosure. In capturing of an intraoral scan, a scanner causes light projector 22 to output a pattern of structured light (e.g., an array of spots), and causes multiple cameras 58 to capture an image of the pattern of structured light projected onto an intraoral surface. Based on the stored calibration values, all projector rays 88 and all camera rays 86 corresponding to all detected spots 33′ are mapped, and all intersections 98 of at least one camera ray 86 and at least one projector ray 88 are identified. As shown in FIG. 8, in an example three projector rays 88 are mapped along with eight camera rays 86 corresponding to a total of eight detected spots 33′ on camera sensors 58 of cameras 24. As shown in FIG. 10, sixteen intersections 98 are identified, where each of the sixteen intersections may be a candidate intersection of a captured point in one of the captured images and a projected point (e.g., of a projector ray). Each candidate intersection 98 defines a three-dimensional point in space. Once candidate intersections are determined, processor 96 may determine a correspondence between projected spots 33 and detected spots 33′ so as to identify a three-dimensional location for each projected spot 33 on the surface, as set forth below with reference to FIGS. 10-34C.

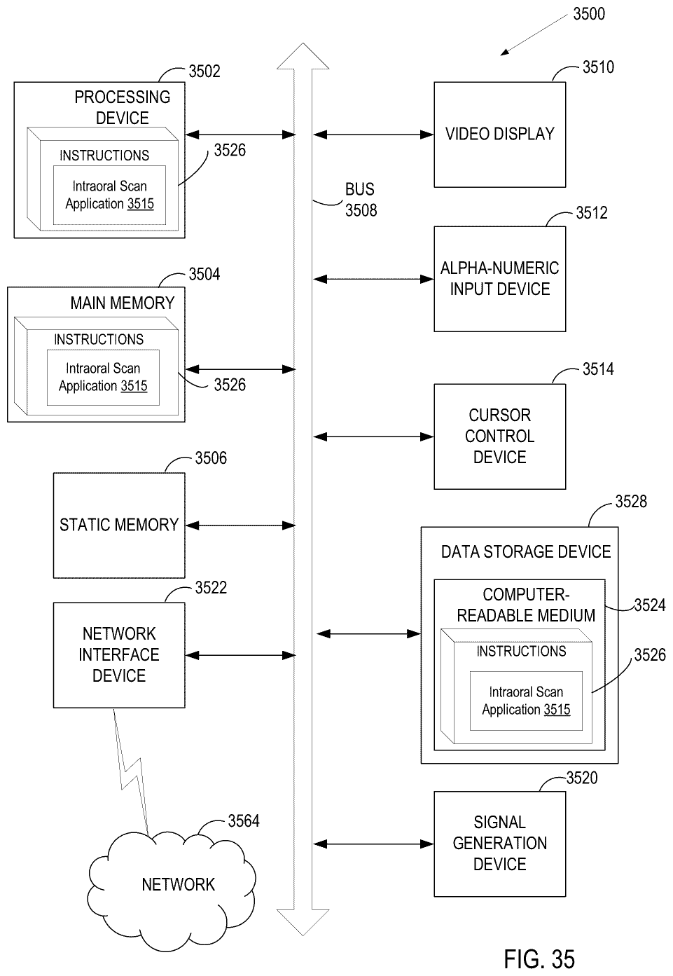

FIGS. 10-34C are flow charts and associated figures illustrating various methods related to determining correspondence of captured points or spots captured by one or more cameras and projected points or spots (e.g., projector rays) projected by one or more structured light projectors. The methods may be performed by a processing logic that may comprise hardware (e.g., circuitry, dedicated logic, programmable logic, microcode, etc.), software (e.g., instructions run on a processing device to perform hardware simulation), firmware, or a combination thereof. In one embodiment, at least some operations of the methods are performed by a computing device of a scanning system and/or by a server computing device (e.g., by computing device 105 of FIG. 1 or computing device 2800 of FIG. 35). In one embodiment, at least some operations of the methods are performed by a processing device of an intraoral scanner (e.g., scanner 150 of FIG. 1).

FIG. 10 is a flow chart for a method 1000 of determining 3D coordinates for points in one or more 2D images, in accordance with embodiments of the present disclosure. At block 1005, processing logic uses one or more trained machine learning models to determine correspondence between captured points (e.g., structured light features) of a captured structured light pattern in one or more images and projected points of a projected light pattern. Each of the captured points may correspond to a pixel location of a camera that generated an image. Each of the projected points may correspond to a projector ray of a structured light projector. In embodiments, each of the captured points is a captured spot and each of the projected points is a projected spot. Alternatively, the captured and projected points may be points of other features of a structured light pattern. For example, captured and projected points may be points on a line, vertices of a geometrical shape, and so on. In some embodiments, different projected and captured points have different colors. For example, a first set of projected and captured points may correspond to a first wavelength and therefore have a first color and a second set of projected and captured points may correspond to a second wavelength and have a second color.

In one embodiment, the one or more machine learning models include one or more neural networks. For example, the one or more machine learning models may be or include one or more deep neural networks, convolutional neural networks, recurrent neural networks, and so on. Other types of machine learning models that may be used include support vector machines, random forest models, k-nearest neighbors models, Bayesian classifiers, logistic regression algorithms, and so on. The one or more machine learning models may include machine learning models trained using supervised learning, semi-supervised learning, unsupervised learning, and/or reinforcement learning. In some embodiments, the one or more trained machine learning models include at least two machine learning models, as discussed further with reference to FIG. 11.

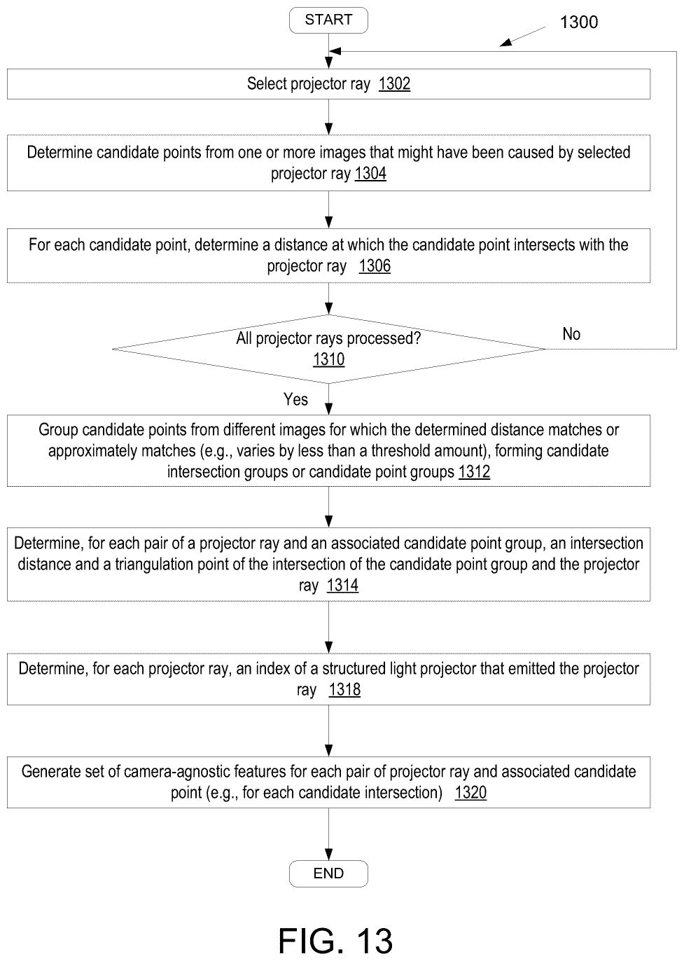

In embodiments, one or more images are generated by an intraoral scanner. The intraoral scanner may include multiple cameras, each of which may capture the same or a different subset of projector points projected by one or more structured light projectors of the intraoral scanner. Multiple images (e.g., each captured by a different camera at a same time) may include captured points that correspond to one or more of the same projected points (e.g., to the same projector rays). The one or more machine learning models may be tasked with determining which of the captured points correspond to which of the projected points (e.g., to which projector rays). This is a non-trivial problem. In embodiments, the one or more machine learning models determine the correspondence between captured points and projector points very quickly (e.g., on the order of micro-seconds or milliseconds) and in real time or near-real time so that this information can be used to construct a 3D point cloud that constitutes an intraoral scan generated at a given time and be registered and stitched to one or more additional intraoral scans previously generated during an intraoral scanning session and/or a 3D surface being constructed as intraoral scanning progresses. In some embodiments, the one or more images are pre-processed prior to inputting data for the intraoral images into the one or more trained machine learning models. The pre-processing may be performed to generate feature vectors or feature data sets (e.g., including properties associated with projected points and/or captured points) that provide useful information from which the one or more machine learning models can a) determine probabilities of captured points in images corresponding to projected points (e.g., projector rays) output by structured light projectors and b) ultimately pair projected points with captured points. Various examples of types of features or properties that are extracted are discussed in greater detail below with reference to FIGS. 13-16.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available. In embodiments, at least some training data includes images and associated 3D point clouds of one or more known objects (e.g., calibration objects). Some training data may include images and associated 3D point clouds (e.g., intraoral scans determined from the images) with labels indicating correspondence between captured points and projector points or projector rays. In some embodiments, the known object may have a known position and/or orientation (e.g., be positioned on a movable stage at a known position and/or orientation), and thus it may be known a priori which captured points correspond to which projector points (e.g., projector rays). Each training data item may include a set of images generated of an object (e.g., a known object) and a label indicating for each captured point in each of the images which projector ray or projector point the captured point corresponds to.

In one embodiment, the one or more machine learning models are trained to output, for one or more candidate intersections of a captured point and a projector ray that might have caused that captured point, a probability that the projector ray caused the captured point. In some embodiments, candidate intersections of multiple images, each captured by a different camera, for which agreement is found for a same or approximate the same distance value for a projector ray are grouped into a candidate intersection group. The one or more machine learning model may additionally be trained to process information for candidate intersection groups, and to output probability scores for such candidate intersection groups. A probability score for a candidate intersection or candidate intersection group, collectively referred to simply as candidates, may be a probability that a distance value of the candidate intersection or candidate intersection group is correct. An output of a machine learning model may be a tuple including probabilities for multiple different candidate intersections. An output of a machine learning model may be a probability for a single candidate intersection or candidate intersection group. Accordingly, one or more machine learning models may consider only a single candidate intersection or candidate intersection group at a time, and one or more other machine learning models may consider multiple candidate intersections and/or candidate intersection groups at a time.

At block 1010, processing logic determines depth information for at least some of the plurality of captured points based on the determined correspondence. Each intersection of a captured point with a projector point (e.g., a projector ray) on a surface of an imaged object may occur at a known depth or distance from the intraoral scanner. Accordingly, if a captured point is determined to have been caused by a particular projector ray, then the depth or distance from the intraoral scanner can be determined. Additionally, the x, y coordinates of each of the captured points are known in each of the images (e.g., based on information about the pixel(s) associated with the captured points). Accordingly, once the depth information is determined a 3D coordinate or location of the intersection of the captured point with the corresponding projected point or projector ray can be determined as well. In this manner 3D coordinates for some or all of the captured points (each having been candidate points for one or more projector rays) in the images can be determined. These 3D coordinates are used to construct a 3D point cloud, which may then be registered to and/or stitched to a 3D surface and/or previously determined 3D point clouds.

FIG. 11 is a flow chart for another method 1100 of determining 3D coordinates for points in one or more 2D images, in accordance with embodiments of the present disclosure. At block 1105 of method 1100, processing logic uses a first trained machine learning model to determine probabilities that captured points of a captured light pattern in one or more images correspond to projected points (which correspond to projector rays) of a projected light pattern. In embodiments, the one or more images are generated by an intraoral scanner. The intraoral scanner may include multiple cameras, each of which may capture the same or a different subset of projector points projected by one or more structured light projectors of the intraoral scanner. Multiple images (e.g., each captured by a different camera at a same time) may include captured points that correspond to one or more of the same projector points (e.g., to the same projector rays).

In some embodiments, the first trained machine learning model is a neural network, such as a deep neural network, a convolutional neural network, a recurrent neural network, and so on. In some embodiments, the first trained machine learning model is a support vector machine, a random forest model, a k-nearest neighbors model, a Bayesian classifier, a logistic regression algorithm, and so on. The first machine learning model may have been trained using supervised learning, semi-supervised learning, unsupervised learning, and/or reinforcement learning.

In some embodiments, the one or more images are pre-processed prior to inputting data for the intraoral images into the first trained machine learning model. The pre-processing may be performed to generate feature vectors or feature data sets (also referred to as property data sets) that provide useful information from which the one or more machine learning models can a) determine probabilities of captured points in images corresponding to projector points (e.g., projector rays) output by structured light projectors and b) ultimately pair projector points with captured points. Various examples of types of properties/features that are extracted are discussed in greater detail below with reference to FIGS. 13-16.

In embodiments, multiple candidate intersections are determined for a set of images, where each image in the set was generated by a different camera at the same time or at approximately the same time. Based on the set of images, processing logic determines all possible candidate intersections, where a candidate intersection is an intersection of a candidate point from an image with a projector ray or projected point. In embodiments, processing logic groups together matching candidate intersections from different images/cameras to generate candidate intersection groups. In embodiments, a feature vector or feature set/property data set may be generated for each candidate intersection and/or candidate intersection group (collectively referred to as candidates). The feature vector or feature set/property data set (which may include one or more of the images and/or may exclude one or more of the images) for a candidate is input into the first trained machine learning model, and the first trained machine learning model outputs a probability score for the candidate (e.g., a probability of a captured point having been caused by a projector ray, which is also a probability of the projector ray having intersected a surface of a dental site at a particular distance from the intraoral scanner). This process may be repeated for each candidate (e.g., for each candidate intersection and/or candidate intersection group). In some embodiments, the probabilities for multiple candidates are determined in parallel (e.g., feature sets for multiple candidates may be input into the first trained machine learning model together, which may provide an output that includes probabilities for each of the candidates).

At block 1110, processing logic uses a second trained machine learning model to determine correspondence between a plurality of captured points and a plurality of projected points (e.g., projector rays that cause the projected points) based on one or more of the determined probabilities determined at block 1105. In some embodiments, an input for the second trained machine learning model includes a set of probabilities for multiple candidates (e.g., for multiple candidate intersections and/or candidate intersection groups). For example, processing logic may select a candidate having a highest probability. The candidate may include a candidate point from one or more image and a projector ray. Processing logic may then determine one or more probabilities for other candidates that also include the candidate point from the one or more image or the projector ray. For example, processing logic may select candidates also associated with the candidate point or projector ray that have next highest probabilities. The input comprising information for the candidates and their associated probabilities (e.g., probability scores) may be input into the second trained machine learning model, which may output a selection of one of the candidates, and therefore of a correspondence between a captured point and a projector ray. This process may be repeated until some criteria is satisfied, wherein with each iteration a new candidate may be selected. In one embodiment, the process is repeated until no remaining candidates can be determined with at least a threshold level of confidence (e.g., with at least an 85% confidence, a 90% confidence, an 80% confidence, etc.).

At block 1115, processing logic determines depth information (and 3D coordinates) for at least some of the plurality of captured points based on the determined correspondence information. Each intersection of a captured point with a projector point (e.g., a projector ray) on a surface of an imaged object may occur at a known depth or distance from the intraoral scanner. Accordingly, if a captured point is determined to have been caused by a particular projector ray, then the depth or distance from the intraoral scanner can be determined. Additionally, the x, y coordinates of each of the captured points are known in each of the images (e.g., based on information about the pixel(s) associated with the captured points. Accordingly, once the depth information is determined, a 3D coordinate or location of the intersection of the captured point with the corresponding projector point or projector ray can be determined as well. In this manner 3D coordinates for some or all of the candidate points in the images can be determined. These 3D coordinates are used to construct a 3D point cloud, which may then be registered to and/or stitched to a 3D surface and/or previously determined 3D point clouds.

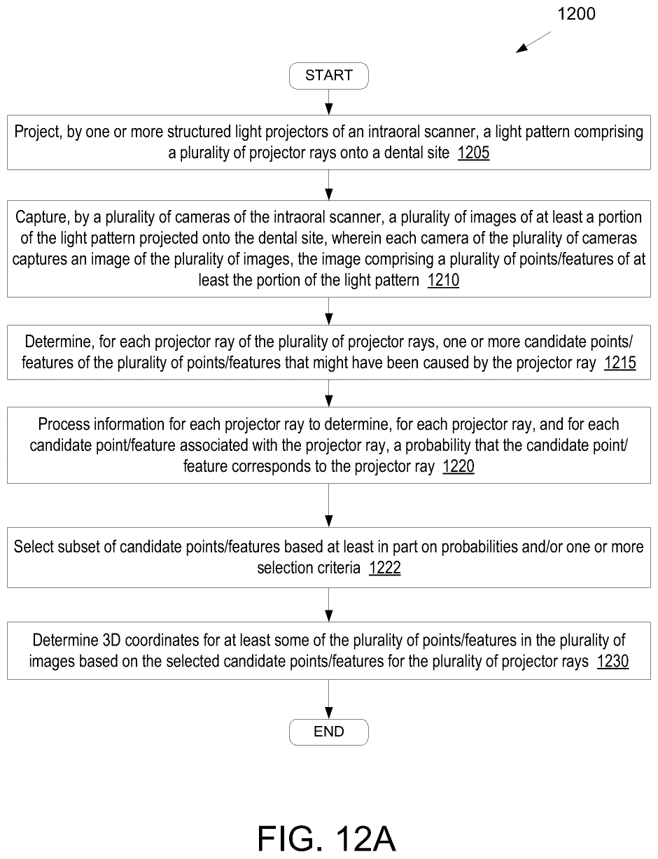

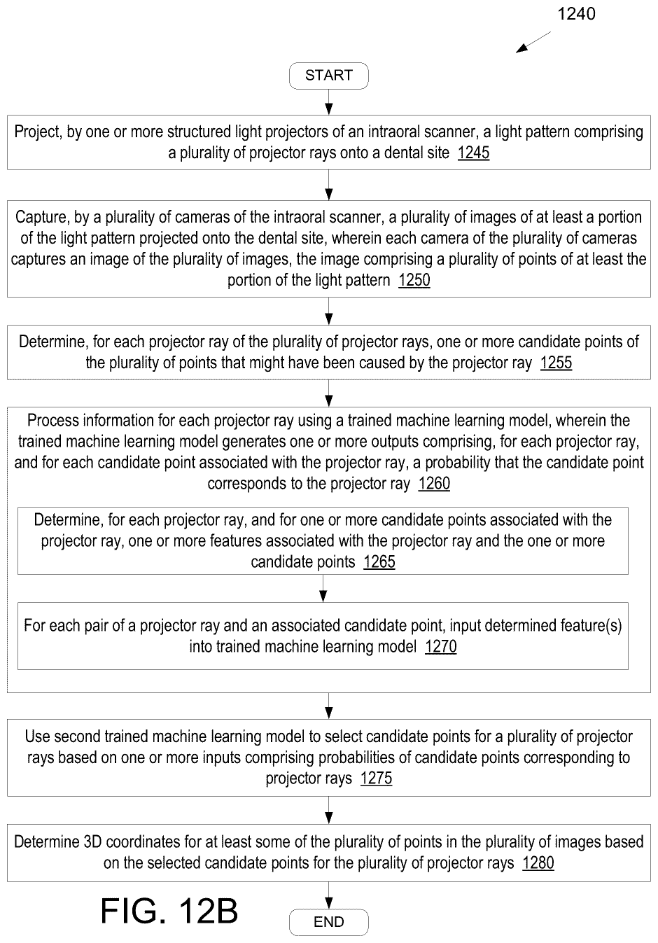

FIG. 12A is a flow chart for another method 1200 of determining 3D coordinates for points in one or more 2D images, in accordance with embodiments of the present disclosure. At block 1205 of method 1200, an intraoral scanner is driven to project, using one or more structured light projectors, a pattern of light comprising a plurality of projector rays onto a dental site (each of which causes a projector point and/or pattern feature when it interfaces with an illuminated surface of the dental site). At block 1210, the intraoral scanner is driven to capture, using one or more cameras, a plurality of images of at least a portion of the light pattern projected onto the dental site. Each camera of the plurality of cameras may capture a distinct image of the dental site at the same time or at approximately the same time, where each of the images comprises a plurality of captured points (e.g., captured structured light features) of at least the portion of the light pattern.

The captured images may be 2D images. However, 3D information may be determined based on the 2D images by matching up captured points/features in the images with projector rays (e.g., projected points or features) output by the structured light projectors. In embodiments, the 2D images are transmitted to a computing device (e.g., computing device 105 of FIG. 1), and one or more subsequent operations are performed at the computing device to extract 3D data from the 2D images and generate a 3D point cloud. Alternatively, a processing device of the scanner 150 of FIG. 1 may extract the 3D information and generate the 3D point cloud, and may transmit the 3D point cloud to computing device 105.

At block 1215, processing logic determines, for each projector ray of the plurality of projector rays of the projected light pattern, one or more candidate points/features in the images that might have been caused by the projector ray. A candidate intersection or pairing may be determined for each pairing of a projector ray and a candidate point/feature of an image.

At block 1220, processing logic processes information for each projector ray to determine probability information for each combination of a projector ray and an associated candidate point/feature (e.g., for each candidate intersection). The probability information for a pair of a projector ray and an associated candidate point/feature that might have been caused by the projector ray (e.g., for a candidate intersection/pairing) may be a probability that the projector ray caused the candidate point/feature. The output of the trained machine learning model may be a probability score (e.g., ranging from a probability of 0% to a probability of 100%). In some embodiments, the probability information is generated using a trained machine learning model (e.g., as described with reference to FIG. 2B). In some embodiments, the probability information is generated using one or more heuristics, statistical processing, rules and/or other techniques that may not apply machine learning.

In one embodiment, processing logic determines, for each projector ray, and for one or more candidate points/features associated with the projector ray (e.g., for each candidate intersection/pairing), one or more properties (also referred to as features) associated with the pairing of the projector ray and the candidate point (e.g., one or more properties associated with the candidate intersection). The properties may include triangulation properties, distance from an epi-polar line, light intensity, spot size, and/or other information. In some embodiments, candidate intersections from multiple images are grouped together to form a candidate intersection group. The candidate intersection group may have a single candidate intersection that is representative of each of the candidate intersections in the candidate intersection group. Properties may be determined for the representative candidate intersection of the candidate intersection group. In some embodiments, determined properties may be divided into camera-agnostic properties and camera-specific properties. A candidate intersection may representative of a candidate intersection group, and may be associated with multiple different cameras/images. Camera-agnostic properties may be shared by each of the images of a candidate intersection group. Each image in a candidate intersection group may be associated with a particular camera, and may have its own unique camera-specific properties for a given candidate intersection. One or more of the various determined properties may be used to determine the probability information in embodiments.

In one embodiment, at block 1222, processing logic selects one or more of the candidate intersections based on the probabilities. A selection of a candidate intersection is a selection of pairing of a projector ray and a candidate point or structured light feature for the projector ray, and provides a depth or distance at which the projector ray interfaced with a 3D intraoral surface. In some embodiments, the candidate intersection selection is performed using a trained machine learning model (e.g., as described with reference to FIG. 2B). In some embodiments, the candidate intersection selection is performed using one or more heuristics, statistical processing, rules and/or other techniques that may not apply machine learning.

At block 1230, processing logic determines depth information (and 3D coordinates) for at least some of the plurality of captured intersections based on the selected candidate intersections for the plurality of projector rays (e.g., based on the selected candidate pairing). Each intersection of a captured point with a projector point (e.g., a projector ray) on a surface of an imaged object may occur at a known depth or distance from the intraoral scanner. Accordingly, if a captured point/feature is determined to have been caused by a particular projector ray, then the depth or distance from the intraoral scanner can be determined. Additionally, the u, v coordinates of each of the captured points are known in each of the images (e.g., based on information about the pixel(s) associated with the captured points. Accordingly, once the depth information is determined, a 3D coordinate or location of the intersection of the captured point/feature with the corresponding projector point/feature or projector ray can be determined as well. In this manner 3D coordinates for some or all of the candidate points/features in the images can be determined. These 3D coordinates are used to construct a 3D point cloud, which may then be registered to and/or stitched to a 3D surface and/or previously determined 3D point clouds.

FIG. 12B is a flow chart for another method 1240 of determining 3D coordinates for points in one or more 2D images, in accordance with embodiments of the present disclosure. At block 1245 of method 1240, an intraoral scanner is driven to project, using one or more structured light projectors, a pattern of light comprising a plurality of projector rays onto a dental site (each of which causes a projector point when it interfaces with an illuminated surface of the dental site). At block 1250, the intraoral scanner is driven to capture, using one or more cameras, a plurality of images of at least a portion of the light pattern projected onto the dental site. Each camera of the plurality of cameras may capture a distinct image of the dental site at the same time or at approximately the same time, where each of the images comprises a plurality of captured points of at least the portion of the light pattern.

The captured images may be 2D images. However, 3D information may be determined based on the 2D images by matching up captured points in the images with projector rays (e.g., projected points) output by the structured light projectors. In embodiments, the 2D images are transmitted to a computing device (e.g., computing device 105 of FIG. 1), and one or more subsequent operations are performed at the computing device to extract 3D data from the 2D images and generate a 3D point cloud. Alternatively, a processing device of the scanner 150 of FIG. 1 may extract the 3D information and generate the 3D point cloud, and may transmit the 3D point cloud to computing device 105.

At block 1255, processing logic determines, for each projector ray of the plurality of projector rays of the projected light pattern, one or more candidate points in the images that might have been caused by the projector ray. A candidate intersection may be determined for each pairing of a projector ray and a candidate point of an image.

At block 1260, processing logic processes information for each projector ray using a trained machine learning model. The trained machine learning model generates one or more outputs containing probability information for each combination of a projector ray and an associated candidate point (e.g., for each candidate intersection). The probability information for a pair of a projector ray and an associated candidate point that might have been caused by the projector ray (e.g., for a candidate intersection) may be a probability that the projector ray caused the candidate point. The output of the trained machine learning model may be a probability score (e.g., ranging from a probability of 0% to a probability of 100%).

In one embodiment, at block 1265 processing logic determines, for each projector ray, and for one or more candidate points associated with the projector ray (e.g., for each candidate intersection), one or more properties (also referred to as features) associated with the pairing of the projector ray and the candidate point(s) (e.g., one or more properties or features associated with the candidate intersection). The properties or features may include triangulation properties or features, distance from an epi-polar line, light intensity, spot size, and/or other information. In some embodiments, candidate intersections from multiple images are grouped together to form a candidate intersection group. The candidate intersection group may have a single candidate intersection that is representative of each of the candidate intersections in the candidate intersection group. Properties or features may be determined for the representative candidate intersection of the candidate intersection group. In some embodiments, determined properties or features may be divided into camera-agnostic properties or features and camera-specific properties or features. A candidate intersection may representative of a candidate intersection group, and may be associated with multiple different cameras/images. Camera-agnostic properties or features may be shared by each of the images of a candidate intersection group. Each image in a candidate intersection group may be associated with a particular camera, and may have its own unique camera-specific properties or features for a given candidate intersection.

In one embodiment, at block 1270, for each pair of a projector ray and an associated candidate point or points (e.g., for a candidate), the determined properties or features are input into the trained machine learning model. The trained machine learning model may determine the probability for the candidate based on the input properties or features.

At block 1275, processing logic may use a second machine learning model to select candidates (e.g., to select candidate points for a plurality of projector rays) based on one or more inputs comprising probabilities of candidate points corresponding to projector rays (e.g., based on probability scores associated with the candidates). In one embodiment, the second machine learning model receives probabilities associated with multiple candidates, and outputs a selection of one of the candidates. This process may be repeated multiple times until no remaining candidates remain that satisfy one or more criteria.

In an example, processing logic may select a candidate having a highest probability. The candidate may include a candidate point from one or more images/cameras and a projector ray. Processing logic may then determine one or more probabilities for other candidates that also include the candidate point from the one or more images/cameras or the projector ray. For example, processing logic may select candidates also associated with the candidate point(s) or projector ray that have next highest probabilities. The input comprising information for the candidates and their associated probabilities (e.g., probability scores) may be input into the second trained machine learning model, which may output a selection of one of the candidates, and therefore of a correspondence between a captured point in one or more images and a projector ray. This process may be repeated until some criteria is satisfied, wherein with each iteration a new candidate (e.g., a new candidate intersection or candidate intersection group) may be selected. In one embodiment, the process is repeated until no remaining candidates can be determined with at least a threshold level of confidence (e.g., with at least an 85% confidence, a 90% confidence, an 80% confidence, etc.).

At block 1280, processing logic determines depth information (and 3D coordinates) for at least some of the plurality of captured points based on the selected candidate points for the plurality of projector rays (e.g., based on the selected candidate. Each intersection of a captured point with a projector point (e.g., a projector ray) on a surface of an imaged object may occur at a known depth or distance from the intraoral scanner. Accordingly, if a captured point is determined to have been caused by a particular projector ray, then the depth or distance from the intraoral scanner can be determined. Additionally, the x, y coordinates of each of the captured points are known in each of the images (e.g., based on information about the pixel(s) associated with the captured points. Accordingly, once the depth information is determined, a 3D coordinate or location of the intersection of the captured point with the corresponding projector point or projector ray can be determined as well. In this manner 3D coordinates for some or all of the candidate points in the images can be determined. These 3D coordinates are used to construct a 3D point cloud, which may then be registered to and/or stitched to a 3D surface and/or previously determined 3D point clouds.

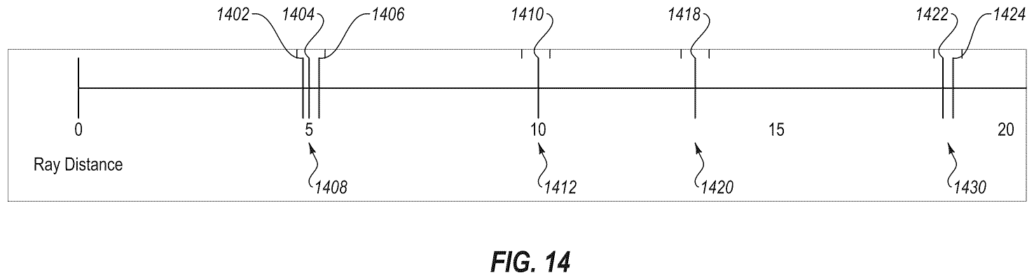

FIGS. 13-14 are flow charts showing preprocessing operations that may be performed to determine candidates (e.g., candidate intersections and/or candidate intersection groups each including one or more pairs of projector rays and candidate points), to determine sets of properties or features for the candidate intersections, and to generate inputs to be provided to one or more trained machine learning models. To solve the correspondence problem of matching projector rays with captured points in images, preprocessing operations may be performed to form candidates (e.g., candidate intersections and/or candidate intersection groups) for the solution of projector ray intersection with the surface of an imaged dental site. Since the projector rays are fixed and known during a calibration process, a 3D point for a projector ray is directly associated with the ray's length (i.e., the length of the ray measuring from projector's origin to intersection with the surface). As a result, the correspondence problem is effectively a 1D problem rather than a 3D problem (e.g., where along the line of the projector ray did an intersection with a 3D surface occur).

Given an intersection of a projector ray with the imaged surface, several cameras might capture the same illuminated point (e.g., a 2D spot) that was generated. Based on calibration data for the cameras and projectors, processing logic can calculate the projector ray's intersection distance for each camera independently by triangulating the projector ray and a camera ray (there is a continuous mapping between a pixel coordinate and it's corresponding camera ray direction). A discussion of how to perform calibration and generate such calibration data is discussed in greater detail in U.S. Application Ser. No. 16/446,181, filed Jun. 19, 2019, which is incorporated by reference herein. Ideally, if processing logic could locate each spot or point to an infinite accuracy, then each camera would have estimated the same projector ray's distance. But in practice a noisy measurement of captured point (e.g., spot) location gives some deviation with statistically higher triangulation error as the ray's distance is high (the triangle's height is much higher than its base).

FIG. 13 is a flow chart for a method 1300 of determining camera-agnostic properties or features associated with points in one or more images (e.g., associated with candidate intersections associated with points in one or more images), in accordance with embodiments of the present disclosure. At block 1302 of method 1300, processing logic selects a projector ray that was generated by a structured light projector. At block 1304, processing logic determines candidate points from the one or more captured images that might have been caused by the selected projector ray. Each pair of a candidate point (e.g., a captured point in an image that might have been caused by the projector ray) and the projector ray may form a candidate intersection. Based on stored calibration data, a distance or depth value may be known for each candidate intersection. At block 1306, processing logic may determine, for each candidate point, a distance at which the candidate point intersects with the projector ray (e.g., may determine the distance from the scanner associated with the candidate intersection).

At block 1310, processing logic determines whether all projector rays have been processed. If not all projector rays have been processed, the method may return to block 1302 for selection of another projector ray. If all projector rays have been processed (e.g., all candidate intersections and their associated distances have been determined for all projector rays), then the method may proceed to block 1312.

As a preprocessing step, processing logic may try to reduce the problem complexity by grouping candidate intersections from different cameras who agree on the same projector ray's distance (roughly). At block 1312, processing logic may group candidate points associated with a same projector ray (e.g., candidate intersections) from different images for which the determined distance matches or approximately matches into a candidate point group (also referred to as a candidate intersection group). Two candidate intersections from different images may be said to match if they vary by less than a threshold amount (e.g., the distance between the points is less than a threshold amount, such as 2, 3 or 4 pixels along one or more axes). In one embodiment, processing logic determines a number of cameras/images associated with the candidate (e.g., number of candidate intersections in a candidate intersection group). The more cameras that agree one a candidate intersection, the higher the likelihood that the candidate intersection is an actual intersection of a projector ray and a captured point.

FIG. 14 illustrates candidate intersections of one or more candidate points with a projector ray, in accordance with embodiments of the present disclosure. The horizontal axis represents distance from the origin of the projector ray. As shown the example projector ray has seven possible intersections (candidate intersections 1402, 1404, 1406, 1410, 1418, 1422, 1424 derived from seven captured points or spots. Processing logic can group candidate points (e.g., candidate intersections each including a candidate point) from different cameras or images to reduce the initial seven candidate intersections to four candidate intersections (e.g., each being a representative candidate intersection for a candidate intersection group) for a solution of the projector ray's intersection distance. A first candidate intersection group 1408 (also referred to simply as a first candidate) is composed from three cameras (each having a candidate intersection 1402, 1404, 1406 for a point or spot having a similar intersection distance for the projector ray) that estimated the projector ray's distance of intersection to be around 5 mm. A second candidate intersection group 1412 (second candidate) may have a single candidate intersection 1410 estimated at around 10 mm. A third candidate intersection group 1420 (third candidate) may have a single candidate intersection 1418 estimated at around 13 mm. A fourth candidate intersection group 1430 (fourth candidate) is composed from two cameras (each having a candidate intersection 1422, 1424 for a point or spot having a similar intersection distance for the projector ray) that estimated the projector ray's distance of intersection to be around 18 mm. Any one of these candidates may correspond to the actual distance from the intraoral scanner to a point on the scanned dental surface.

Returning to FIG. 13, at block 1314, for each candidate, a ray intersection distance may be determined. The ray intersection distance may be determined by averaging the ray intersection distances of each of the candidate intersections in the candidate intersection group. The average may be a weighted average or a non-weighted average. Once the ray intersection distance is determined, a triangulation point of the candidate may be determined. The triangulation point may be a 3D location (e.g., x, y, z) of the triangulation.

As previously mentioned, the intraoral scanner may include multiple structured light projectors. Different structured light projectors may output a different light pattern, may output light having a different wavelength, may output a light pattern having differently shaped spots, etc. Each projector ray may be associated with a structured light projector that produced that projector ray. At block 1318, processing logic may determine an index of the structured light projector for each projector ray, and may add the index to each of the candidate intersections/candidate intersection groups (candidates) associated with the projector ray.

At block 1320, processing logic generates a set of camera-agnostic properties or features for each pair of a projector ray and associated candidate point (e.g., for each candidate intersection or candidate intersection group). The set of camera-agnostic properties or features may include, for example, distance from intraoral scanner, 3D location of the intersection (triangulation point), index of the associated structured light projector, and so on. The set of camera-agnostic properties or features associated with a candidate intersection group may be used to form an input for the first trained machine learning model in embodiments.

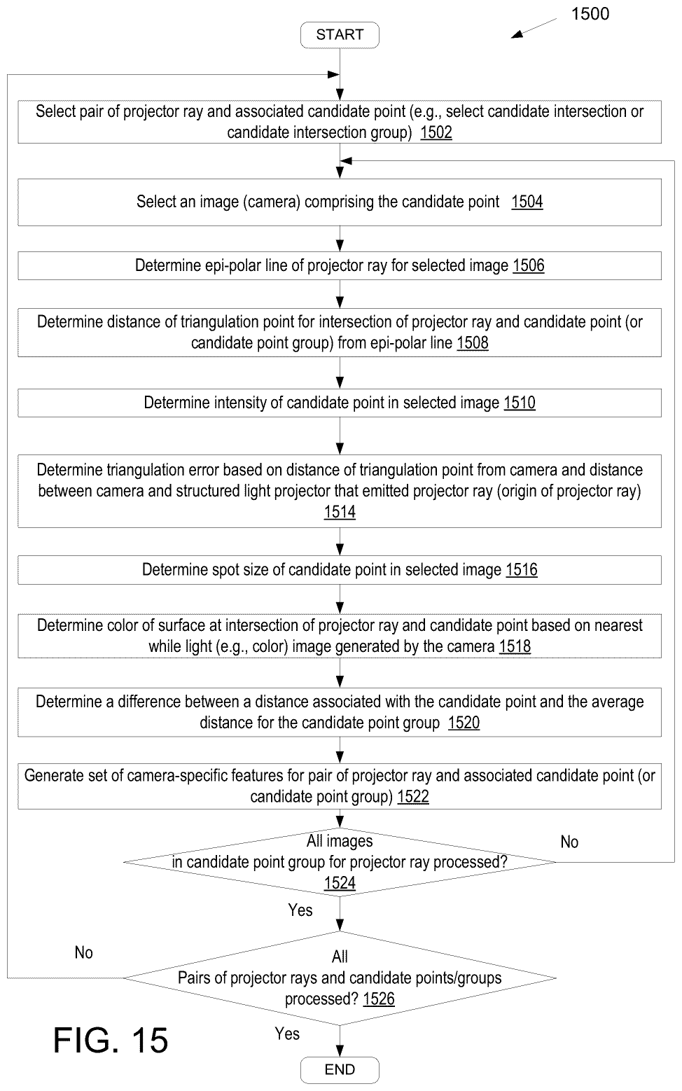

FIG. 15 is a flow chart for a method 1500 of determining camera-specific properties or features associated with points in one or more images, in accordance with embodiments of the present disclosure. In embodiments, method 1500 is performed after method 1300, and relies on at least some of the information determined in method 1300 for determination of one or more camera-specific properties or features.

At block 1502 of method 1500, processing logic selects a pair of a projector ray and an associated candidate point. In other words, processing logic selects a candidate (e.g., a candidate intersection or a candidate intersection group). At block 1504, processing logic selects an image (which corresponds to a camera) comprising the candidate point (e.g., the candidate point of the candidate intersection or the candidate intersection group). At block 1506, processing logic determines an epi-polar line for the projector ray for the selected image. The epi-polar line may be a line representing the projector ray's path as viewed by the selected camera. For example, line 92 in FIG. 6A is an ep-polar line of projector ray 88. The further a captured point is from the epi-polar line, the lower the likelihood that the captured point was caused by the projector ray associated with that epi-polar line. Accordingly, distance from the epi-polar line is useful information to determining a probability that a projector ray caused a captured point. At block 1508, processing logic determines a distance of the triangulation point for the intersection of the projector ray and the candidate point from the epi-polar line (e.g., the distance of the captured point of the candidate intersection from the epi-polar line). The determined distance may be an orthogonal distance to the epi-polar line, or a distance from a particular target intersection point on the epi-polar line (e.g., based on a known point at which the particular projector ray should intersect with a particular camera ray).

At block 1510, processing logic may determine an intensity of the captured point in the selected image (e.g., for the selected camera). Intensity of projected light decreases with distance. Accordingly, there is a rough correlation between distance and intensity which can be used by the first trained machine learning model to assist an estimation of a probability that a particular captured point was caused by a particular projector ray.

At block 1514, processing logic may determine a triangulation error based on a distance of the triangulation point from the camera and a distance between the camera and the structured light projector that emitted the projector ray (e.g., an origin of the projector ray). Processing logic may determine a distance between the camera and the structured light projector (or between the pixel in the camera that captured the point and the origin of the ray in the structured light projector). Processing logic may also determine the distance between the intraoral scanner and the candidate intersection. The distance may be a distance to the camera or an orthogonal distance to a line between the camera and the structured light projector. Processing logic may determine a triangulation angle for the candidate intersection based on the distance to the point of intersection and the distance between the camera that captured the point and the structured light projector that generated the point. The lower the distance to the point and the greater the distance between the camera and projector, the greater the triangulation angle and thus the greater the accuracy of the determined distance to the candidate intersection. Accordingly, the accuracy of the candidate intersection may be directly proportional to the distance between the camera and projector and may be inversely proportional to the distance to the candidate intersection. In one embodiment, an error for the determined distance to the candidate intersection may be determined using the function e=z²/bf, where e is an error for the determined distance to the point, z is the determined distance to the point, f is the focal length of the cameras, and b is the base (the distance between the camera and projector). In embodiments, the error associated with the triangulation angle of the point is determined, and the error is used as a feature/property to be included in an input for the candidate intersection to a trained machine learning model.

At block 1516, processing logic determines a spot size of the captured point in the image that is associated with the candidate. In embodiments, spot size is a function of distance from the intraoral scanner. The size of a spot or other structured light feature generally increases with distance from the intraoral scanner. Processing logic may include calibration data indicating the approximate spot/feature size that should be detected at various distances. Alternatively, such information may be derived by the machine learning model during training of the machine learning model. For example, spots projected onto a surface at a first relatively close distance may have a first general spot size, spots projected onto a surface at a second distance that is greater than the first distance may have a second larger general spot size, and spots projected onto a surface at a third distance that is greater than the second distance may have a third even larger general spot size.

At block 1518, processing logic may determine a color of the surface at an intersection of the projector ray and the candidate point for the image based on one or more nearest white light (e.g., color) image generated by the camera. The intraoral scanner may alternate between generation of 2D images while structured light of one or a few wavelengths is projected and generation of 2D images while white light is projected. 2D images captured during projection of white light provide color information. A pixel or pixels associated with the captured point in the selected image may be determined. The color for the same pixel or pixels in a white light 2D image generated by the camera before and/or after generation of the image using structured light may also be determined. An assumption may be made that the intraoral scanner did not move or moved only minimally between capturing of the structured light in the image and the capturing of the white light in a subsequent and/or previous image. Accordingly, the color of the pixel(s) in the white light image may be assigned to the candidate point for the selected image (and of the candidate comprising the candidate point). The color information may be used to help determine probability of a candidate that includes the candidate point. For example, if a candidate includes candidate points from multiple cameras, but those cameras disagree on the color of the surface at the intersection, this may lower a confidence in the candidate.

At block 1520, processing logic may determine a difference between a distance associated with the candidate point (e.g., a distance of the candidate intersection or candidate intersection group that includes the candidate point) and the average distance for the candidate intersection group (also referred to as candidate point group). If a candidate point is part of a candidate intersection group and its distance of intersection is very close to the average distance of intersection to the candidate intersection group, then the confidence for the candidate intersection group may increase. However, if a candidate point is part of a candidate intersection group and its distance of intersection is not as close to the average distance of intersection for the candidate intersection group, then the confidence for the candidate intersection group may decrease.

At block 1522, processing logic generates a set of camera-specific properties or features for the pair of projector ray and associated candidate point (or candidate point group). In other words, processing logic may generate a set of camera-specific properties or features for a candidate intersection or candidate intersection group.

At block 1524, processing logic determines whether all images in the candidate point group or candidate intersection group have been processed. If not all images in the candidate point group or candidate intersection group have been processed, the method returns to block 1504 and another image is selected for the candidate intersection group. If all images have been processed for the candidate intersection group, the method continues to block 1526.

At block 1526, processing logic determines whether all pairs of projector rays and candidate points/candidate point groups (e.g., whether all candidate intersections or candidate intersection groups) have been processed. If so, the method ends. If not, the method returns to block 1502 and another candidate intersection or candidate intersection group is selected.

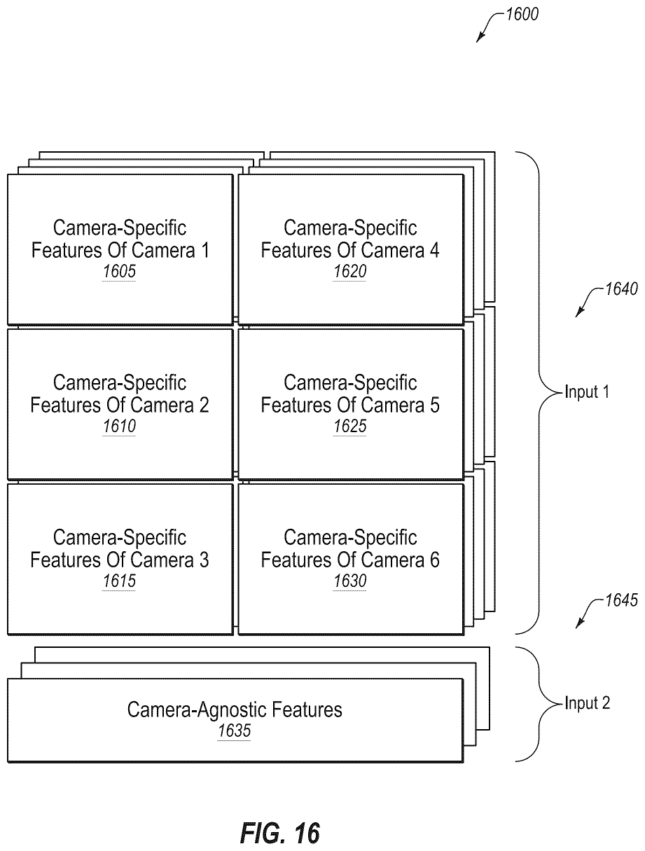

FIG. 16 illustrates information 1600 associated with a candidate (e.g., a candidate intersection or candidate intersection group) for input into a trained machine learning model, including a set of camera-specific properties or features and a set of camera-agnostic properties or features, in accordance with embodiments of the present disclosure. The information 1600 includes a set of camera-specific properties or features 1640 for each candidate and a set 1645 of camera-agnostic properties or features 1635 for each candidate. In the example that there are six cameras, each set of camera-specific properties or features may include information for up to six cameras (e.g., if six cameras saw the same point), such as camera-specific properties or features of camera 1 1605, camera-specific properties or features of camera 2 1610, camera-specific properties or features of camera 3 1615, camera-specific properties or features of camera 4 1620, camera-specific properties or features of camera 5 1625, and/or camera-specific properties or features of camera 6 1630. If a particular camera failed to see a particular projected point (e.g., is not included in a candidate intersection), then camera-specific properties or features for that camera may include zeros or null values in embodiments.

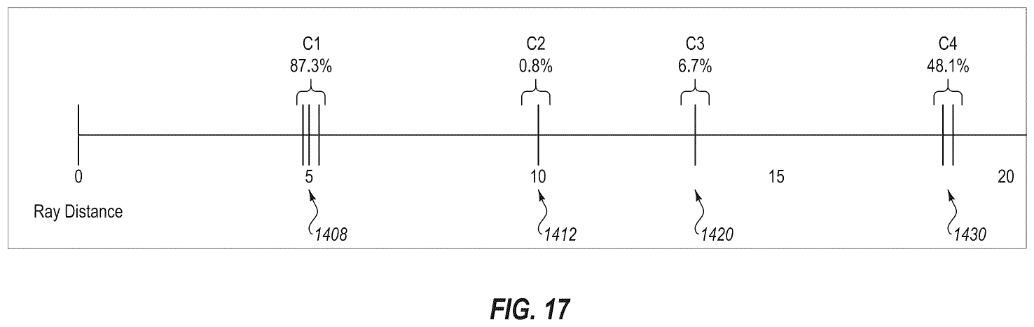

After information associated with each particular candidate (e.g., each combination of a possible intersection of a projector ray and a candidate point from one or more images) is processed by the first trained machine learning model (e.g., at block 1220 of method 1200), probabilities of the projector ray having caused each of the candidate points (e.g., probability scores of each candidate) may be provided. FIG. 17 illustrates the candidate intersections of FIG. 14 with the addition of added probability information as output by a trained machine learning model, in accordance with embodiments of the present disclosure. As shown, first candidate intersection group 1408 (which is a combination of three candidate intersections) has a probability of 87.3%, second candidate intersection group 1412 (which is a single candidate intersection) has a probability of 0.8%, third candidate intersection group 1420 (which is a single candidate intersection) has a probability of 6.7%, and fourth candidate intersection group 1430 (which is a combination of two candidate intersections) has a probability of 48.1%. In embodiments, the machine learning model that outputs probability scores outputs such probability scores indifferent to probability scores output for other candidate intersections that might be associated with the same projector ray. For example, a sum of the probabilities of the different candidate intersection groups 1408-1430 of FIG. 17 equals 142.9%, which is greater than 100%.

Once probabilities are determined for candidates (e.g., candidate intersections and/or candidate intersection groups), processing logic may use that probability information to select candidates (e.g., to select which candidate points in images were caused by which projector rays). In some embodiments, such a selection process is performed using a second trained machine learning model, such as at block 1275 of FIG. 12B.

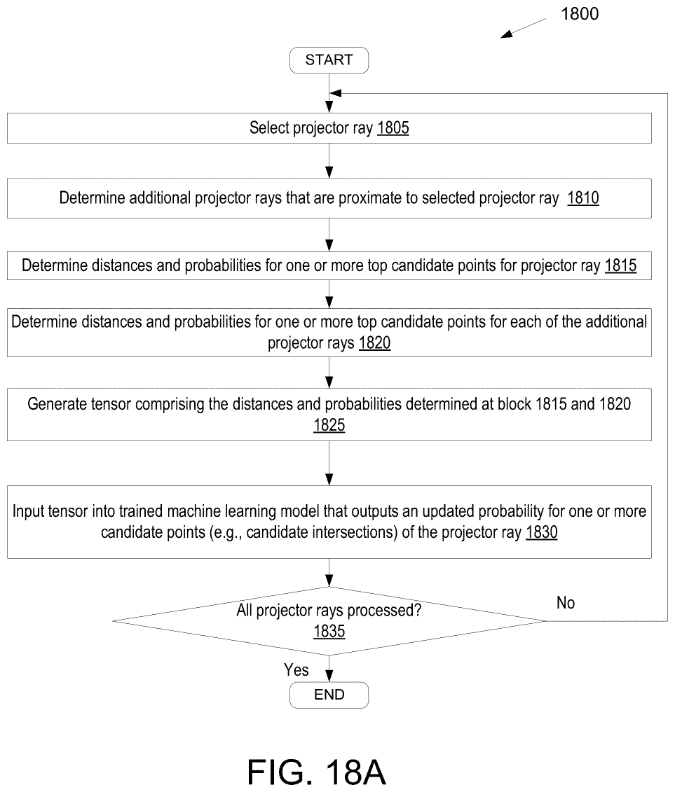

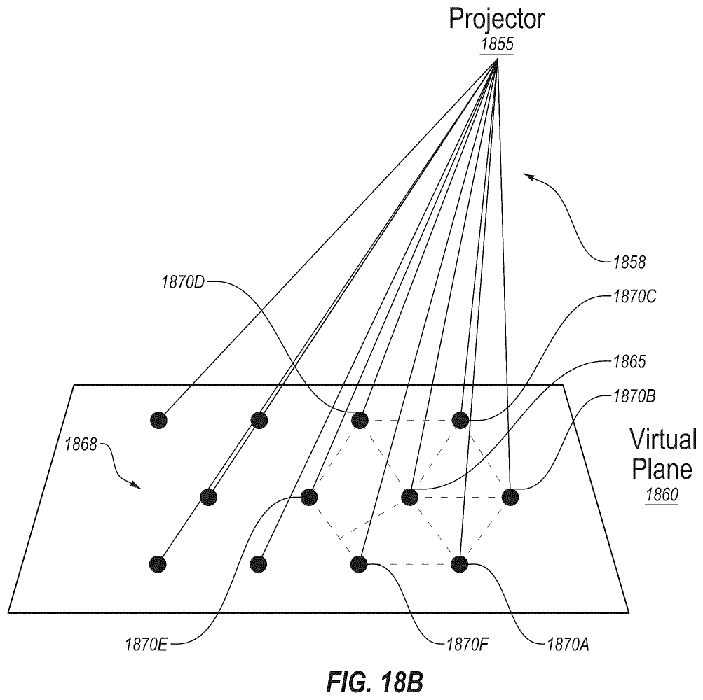

FIG. 18A is a flow chart for a method 1800 of adjusting probabilities for candidates associated with a projector ray based on information about candidates associated with one or more proximate projector rays, in accordance with embodiments of the present disclosure. Processing logic may examine candidates associated with proximate rays that are proximate to a ray in question to determine if the neighboring rays provide hints about the surface illuminated by the rays. If a surface continuity is assumed (e.g., that nearby rays will have similar intersection distances), then large variations in intersection distances for rays between a candidate ray and on or more proximate rays may indicate that a probability score of candidate for the ray in question should be reduced. In some embodiments, method 1800 is performed based on the output of block 1220 of method 1200.

At block 1805 of method 1800, processing logic selects a projector ray. At block 1810, processing logic determines one or more additional projector rays that are proximate to the selected projector ray.

FIG. 18B illustrates an example of proximate projector rays, in accordance with embodiments of the present disclosure. In particular, FIG. 18B shows a structured light projector 1855 projecting a light pattern comprising an array of projector rays 1858. The array of projector rays 1858, when intersecting a virtual plane 1860 form a pattern of disconnected spots 1868 on the virtual plane 1860. For any given spot (e.g., spot 1865) proximate spots 1870A-F may be determined. In the illustrated example, projector 1855 projects projector rays that form a hexagonal pattern of spots. Accordingly, a spot may have up to six proximate spots arrayed around that spot.

Returning to FIG. 18A, at block 1815 processing logic determines distances and probabilities for one or more (e.g., n) top candidate points for the selected projector ray. Each top candidate point may correspond to a candidate having a highest to nth highest probability.

At block 1820 processing logic determines distances and probabilities for one or more (e.g., n) top candidates (e.g., each including a candidate point from one or more images) for each of the additional projector rays, where there may be up to k additional projector rays considered. Each top candidate may correspond to a candidate having a highest to nth highest probability. Such information may be in the form of candidates comprising the candidate points and associated probabilities as output by a trained machine learning model.

At block 1825, processing logic generates a tensor comprising the distances determined at block 1820 and the probabilities determined at block 1820. Assuming that n top candidates are selected for k+1 rays (the selected ray and k neighboring rays), the tensor may be a (k+1)×n×2 tensor. At block 1830, processing logic inputs the tensor into a trained machine learning model (e.g., a neural network), which outputs an updated probability for one or more candidate points of the projector ray. Different rays can have a different number of candidates. In embodiments the tensor input into the trained machine learning model may be standardized, such that a set number of highest scoring candidates is used regardless of the number of candidates for a given projector ray. The value of n may be selected based on a tradeoff between accuracy and performance (e.g., CPU load). The output of the trained machine learning model may be an updated probability for one or more candidates of the selected projector ray. Alternatively, the output of the trained machine learning model may be a weight (e.g., adjustment factor) to apply to the probability scores for one or more candidates associated with the projector ray.

At block 1830, processing logic determines whether or not all projector rays have been processed. If there are remaining projector rays that have not yet been processed, the method returns to block 1805 and a new projector ray is selected. If all projector rays have been processed, the method ends.

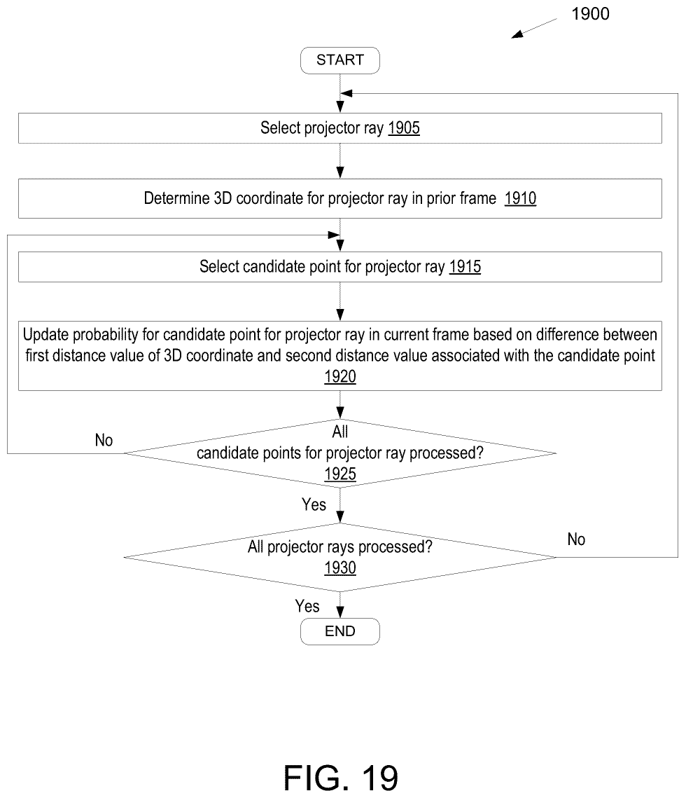

FIG. 19 is a flow chart for a method 1900 of adjusting probabilities for candidates associated with a projector ray for a current frame based on information about the projector ray from one or more prior frames, in accordance with embodiments of the present disclosure. Processing logic may examine candidates selected for one or more prior frames to adjust probability values of candidates for a current frame in embodiments. Each frame may correspond to a point in time at which a set of images was generated. The intraoral scanner may generate images periodically or continuously at a given frame rate, and each time interval at which images are generated may correspond to a frame. An assumption may be made that consecutive frames will likely have the same or similar intersection distances for the same projector rays due to a high frame rate of the intraoral scanner (e.g., a frame rate of over 60 Hz or about one image generated about every 16 or 17 ms). In some embodiments, method 1900 is performed based on the output of block 1220 of method 1200 for a current frame and the output of block 1225 and/or 1230 for one or more prior frame.

At block 1905 of method 1900, processing logic selects a projector ray. At block 1910, processing logic determines a 3D coordinate (or just a distance value) for the selected projector ray in one or more prior frames.

At block 1915, processing logic selects a candidate point for the selected projector ray (e.g., a candidate intersection for the projector ray). At block 1920, processing logic updates the probability for the candidate point for the projector ray in the current frame based on a difference between a first distance value of the 3D coordinate for the projector ray in the prior frame(s) and the distance value associated with the candidate point (e.g., candidate intersection) in the current frame. In one embodiment, the candidate intersection score is updated according to the following normal distribution:

$$f ( x ) = \frac{1}{σ \sqrt{2 π}} {e}^{- \frac{1}{2} {( \frac{x - μ}{σ} )}^{2}}$$

Where μ is the previous estimated ray distance and x is the current candidate's ray distance. In embodiments, the candidate score is not updated if the ray was not solved in the previous frame.

At block 1925, processing logic determines whether all candidate points (candidate intersections) for the selected projector ray have been processed. If there are remaining candidates that have not yet been processed, the method returns to block 1915 and a new candidate is selected. If all candidate points for the projector ray have been processed, the method continues to block 1930.

At block 1930, processing logic determines whether or not all projector rays have been processed. If there are remaining projector rays that have not yet been processed, the method returns to block 1905 and a new projector ray is selected. If all projector rays have been processed, the method ends.

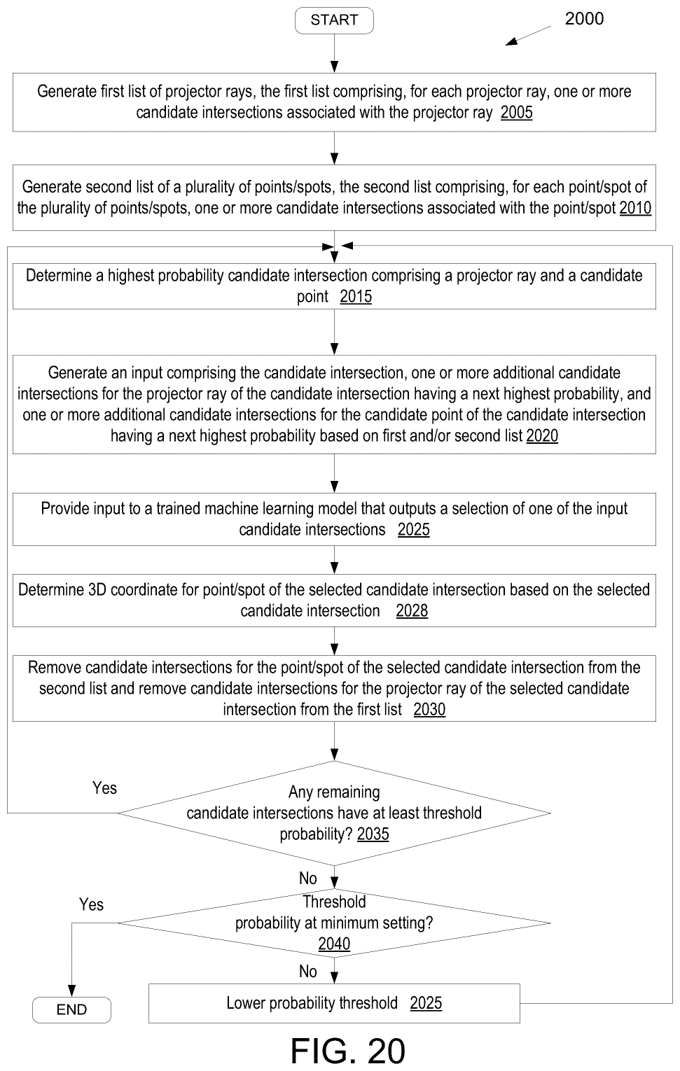

FIG. 20 is a flow chart for a method 2000 of determining 3D coordinates for points in one or more 2D images using a trained machine learning model, in accordance with embodiments of the present disclosure. In embodiments, method 1800 is performed at block 1110 of method 1100 or at block 1225 of method 1200.

Once processing logic has determined a probability score for each candidate (e.g., for each candidate intersection or each candidate intersection group) of a given ray, processing logic needs to choose the correct candidate intersection as the actual intersection of the projector ray with an imaged surface (if there any). The correct candidate is not necessarily the candidate that has the highest probability score. According to the manner in which the first machine learning model that outputs probability scores for candidate intersections, the predicted probability scores should provide a probability, for each candidate, of that given candidate being the correct solution. The purpose of candidate selection is therefore to choose among all probability scores the best combination of candidates, from the entire selection of options and combinations. This amounts to a global optimization of selecting candidate intersections that in the aggregate have a highest combined probability score. However, the probability score for any given individual projector ray may not be maximized.

Solving the entire correspondence all together is quite difficult. Additionally, candidates may be candidate intersection groups (e.g., groups of 2D spots), and those spots in any given group may participate in several different candidate intersection groups. Accordingly, in embodiments processing logic restrains the spots to participate in just one candidate. That is, once processing logic chooses a specific candidate, processing logic eliminates all the spots of the candidate from the rest of the unsolved candidates. To do that, processing logic solves for candidate selection in a greedy manner in which it prioritizes candidates according to their score, and then updates the rest of the candidates once a candidate has been selected (e.g., once a distance for a projector ray is solved and/or a correspondence between a projector ray and a candidate point is determined).

In an embodiment, the entire spots, candidates, and projector rays can be summarized in two tangled tables that associate from spots (or captured points) to projector rays via candidates (e.g., candidate intersection groups and/or candidate intersections) as an intermediate step, such that (Rays↔Candidates↔Spots).

In one embodiment, at block 2005 processing logic generates a first table or list of projector rays. The first list may comprise, for each projector ray, one or more candidates associated with the projector ray and their associated probability scores. At block 2010, processing logic generates a second list or table of the plurality of points or spots. The second list may comprise, for each point or spot of the plurality of points or spots, one or more candidates associated with the point or spot. The pair of tables may be as follows:
1) Rays↔Candidates (which associated with score);
2) Candidates↔Spots (which associated with on/off, true/false).

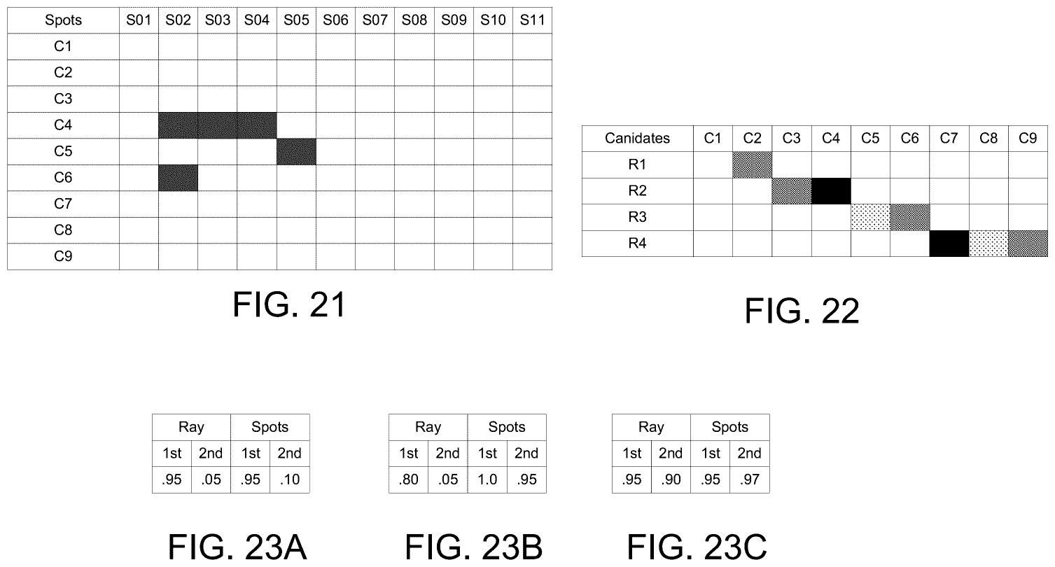

FIG. 21 illustrates an example table of a plurality of points in one or more images, the table comprising, for each point of the plurality of points, one or more candidates associated with the point, in accordance with embodiments of the present disclosure. As shown, the table of FIG. 21 is an example of the Candidates↔Spots table, and each entry is a true or false entry. In other words, a spot is either a member of a candidate or is not a member of the candidate. A black square/rectangle indicates that a spot is a member of a candidate, and a white square/rectangle indicates that a spot is not a member of a candidate. As shown, each captured spot or point may correspond to multiple candidates. Additionally, multiple different captured spots or points may correspond to the same candidate.

FIG. 22 illustrates an example table of a plurality of projector rays, the table comprising, for each projector ray of the plurality of projector rays, one or more candidates associated with the projector ray, in accordance with embodiments of the present disclosure. As shown, the table of FIG. 22 is an example of the Rays ↔Candidates table, and each entry includes a probability value. In the illustrated example, different hatching represents different ranges of probability scores. Darker hatching represents higher probability scores, while lighter hatching represents lower probability scores and no hatching represents a zero probability. A single projector ray may belong to multiple candidates, and may have different probability scores for different candidates.

Returning to FIG. 20, at block 2015 processing logic determines a highest probability candidate comprising a projector ray and a candidate point. The highest probability candidate in the table of FIG. 22 may be, for example, the entry for projector ray R2 and candidate C4 (R2-C4) or the entry for projector ray R4 and candidate C7 (R4-C7).

At block 2020, processing logic generates an input for a trained machine learning model (e.g., for the second trained machine learning model used at block 1225 of method 1200. The generated input may include the candidate and its associated probability score as well as one or more additional candidates and their associated probability scores. The one or more additional candidates may be those candidates for the projector ray of the candidate having a next highest probability score and/or those candidates for the captured point or spot of the candidate having a next highest probability score.

In the example of FIG. 21, it can be seen that spots S2, S3 and S4 are included in candidate C4. It can also be seen that S2 is also a participant in C6. An easy decision would be if all of these spots were only participants in C4. However, it is a tougher decision when one or more spots are participants of multiple candidates.

In one embodiment, the input associated with a candidate that is prepared for the machine learning model includes n highest probability scores associated with the projector ray of the candidate and n highest probability scores associated with the captured point or spot of the candidate. In one embodiment n is equal to two, which may result in four values being included in the input, where two of the values are the same (e.g., two instances of the probability score of the candidate intersection). Three different examples of inputs for the machine learning model are provided in FIGS. 23A-C.

FIGS. 23A-C illustrate example input data sets for input into a trained machine learning model, in accordance with embodiments of the present disclosure. FIG. 23A illustrates an easy example, in which the probability score for the candidate is the highest probability score by far for the spot and ray (e.g., the spot and ray mutually favor each other with high probability). FIG. 23B illustrates a moderately difficult example, in which the probability score for the candidate is the highest probability score, but there is also a close probability score for another spot. FIG. 23B illustrates a difficult example, in which the probability score for the candidate is not the highest probability score for the spots.

Returning to FIG. 20, at block 2025 the generated input is input into the trained machine learning model, which outputs a selection of one of the input candidates. The selection may or may not be the candidate that had the highest probability score (e.g., that represented a local maxima).

At block 2028, processing logic may determine a 3D coordinate for the captured point (e.g., spot) of the selected candidate.

At block 2030, processing logic removes candidate intersections for the point/spot of the selected candidate intersection from the second table or list and removes the candidate intersections for the projector ray of the selected candidate intersection from the first table or list.

At block 2035, processing logic determines whether any remaining candidates have at least a threshold probability score. If so, the method returns to block 2015, and a next highest probability candidate is selected. If no remaining candidates have at least the threshold probability score, the method continues to block 2040. At block 2040, processing logic determines whether the probability threshold is currently set to a lowest possible setting for the probability threshold. If so, the method ends. If the current probability threshold is not set to the lowest setting for the probability threshold, the method continues to block 2025. At block 2025, processing logic lowers the probability threshold. The method then returns to block 2015, and a candidate intersection having a next highest probability score is selected.

Processing logic may solve the correspondence in a descending order starting from the most confident candidates, and may determine candidate selection for candidates associated with the highest probability scores until there are no remaining candidates with scores meeting an initial probability threshold. Processing logic may then incrementally lower the probability threshold, and repeat the process one or more times using the lowered probability threshold. This process may be repeated one or more additional times until the probability threshold has been lowered to a minimum setting for the probability threshold. Proceeding in this fashion ensures that selections are initially made with the highest confidence, which eliminates some possibilities and may increase the accuracy of selection for further candidates that might have lower probability scores. Each time processing logic solves some candidates, it updates the lists (removing the solved candidates from the lists).

To get computation as fast as possible, in one embodiment processing logic divides the minimal allowed threshold into three evenly separated thresholds (e.g., 97.5%, 95%, 92.5%).

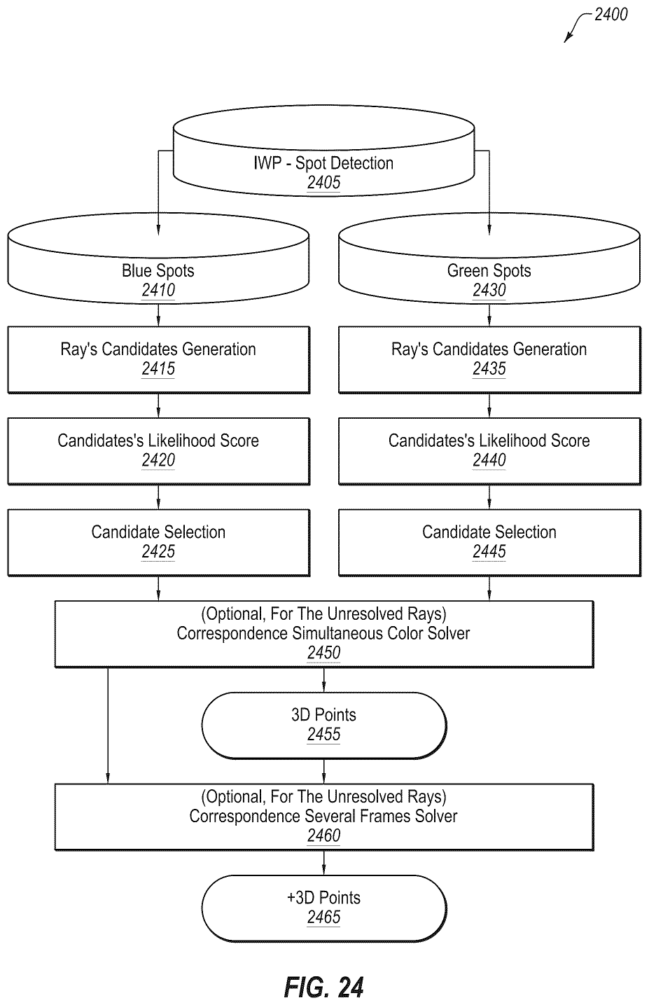

FIG. 24 is a flow chart for a method 2400 of determining 3D coordinates for points in one or more 2D images, in accordance with embodiments of the present disclosure. At block 2405 of method 2400, processing logic performs spot detection on a set of received 2D images of a dental site illuminated by patterned light from one or more structured light projectors. In some embodiments, the structured light projectors include first structured light projectors that output blue spots and structured light projectors that output green spots. Data for blue spots and green spots may be separated and processed separately in embodiments. Processing logic may divide the detected spots in captured images into blue spots 2410 and green spots 1430 in embodiments.

In one embodiment, at block 2415 for blue spots processing logic performs candidate generation for projector rays and at block 2435 processing logic performs candidate generation for green spots. Candidate generation may be performed by using calibration data to match up captured spots in captured 2D images with projected spots (each corresponding to a projector ray). Spot detection yields many candidate intersections of projected spots and captured spots.

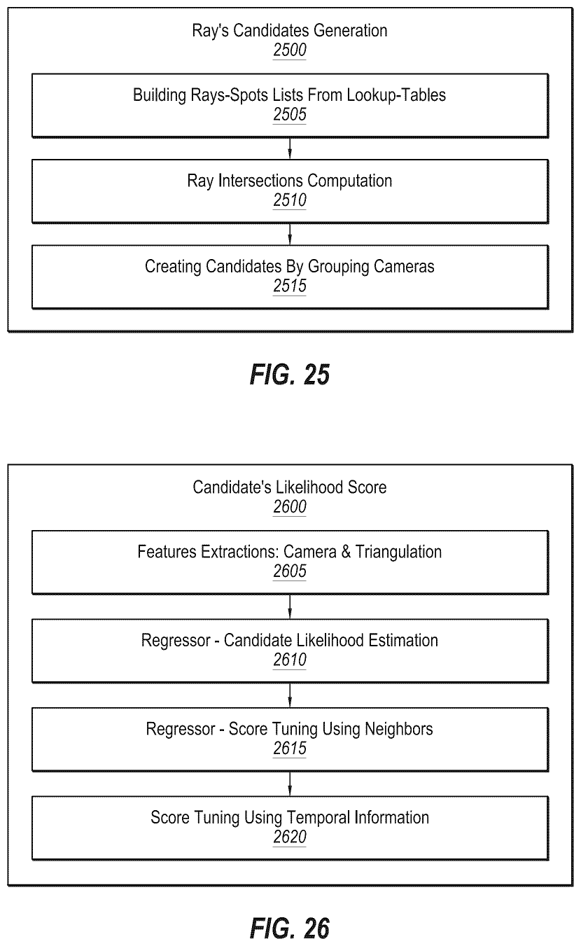

FIG. 25 is a flow chart for a method 2500 of determining candidate spots (or other types of points or structured light features) for projector rays, in accordance with embodiments of the present disclosure. Method 2500 may be performed at both blocks 2415 and 2435 of method 2400 in embodiments. At block 2505 of method 2500, processing logic builds lists of possible ray and spot groupings from lookup tables. At block 2510, processing logic computes all possible candidate intersections of projector rays and captured spots for the set of 2D images. At block 2515, processing logic groups together candidate intersections into candidate intersection groups (also referred to simply as candidates).

Returning to FIG. 24, at block 2420 processing logic computes the probability score (also referred to as likelihood score) for each of the candidates for the blue spots. Similarly, at block 2440 processing logic computes the probability score for each of the candidates for the green spots.

FIG. 26 is a flow chart for a method 2600 of determining probabilities of candidate points being caused by projector rays, in accordance with embodiments of the present disclosure. Method 2600 may be performed at both blocks 2420 and 2440 of method 2400 in embodiments. At block 2605 of method 2600, processing logic performs feature extraction to determine camera-specific properties/features and camera-agnostic properties/features for each of the determined candidates. At block 2610, a trained machine learning model (e.g., a regressor) uses the extracted properties/features to determine a probability score for each candidate (e.g., for each green spot candidate or for each blue spot candidate). At block 2615, processing logic may use a second trained machine learning model to adjust the probability scores of candidates for spots based on distance information for neighboring spots and/or probability scores for candidates of neighboring spots. At block 2620, processing logic may further adjust or tune probability scores for candidates based on distance information for associated projector rays from past frames.

Returning to FIG. 24, at block 2425 processing logic performs candidate selection for the blue spots. Similarly, at block 2445 processing logic performs candidate selection for the green spots.

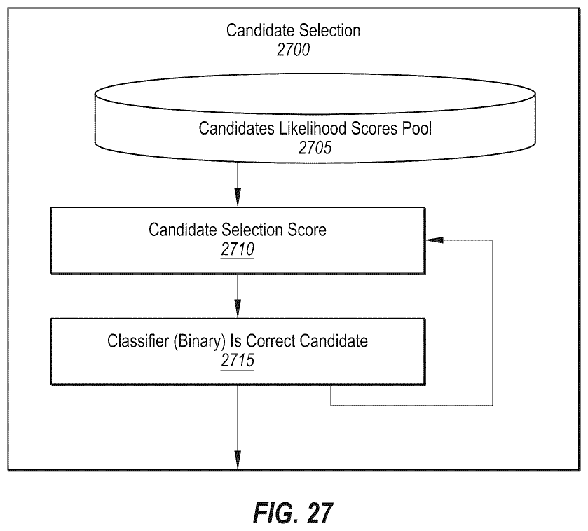

FIG. 27 is a flow chart for a method 2700 of selecting candidate points (e.g., spots) for projector rays, in accordance with embodiments of the present disclosure. At block 2705 of method 2700, processing logic may generate a candidate probability score pool. In one embodiment, projector rays are ranked based on the highest scoring candidate probability score associated with the projector ray. In one embodiment, candidates are ranked based on their probability scores. From the candidate probability score pool, at block 2710 processing logic performs a candidate selection score calculation. This may include selecting a highest scoring candidate, determining a projector ray and a candidate point (e.g., a candidate spot) associated with the highest scoring candidate, and further selecting one or more other highest scoring candidates that share the same projector ray and selecting one or more other highest scoring candidates that share the same spot. A feature set/property set comprising the selected candidates and their associated probability scores is generated. At block 2715, the feature set/property set comprising the selected candidates and their associated probability scores is input into a trained machine learning model, which outputs a selection of one of the input candidates. The method then loops back to block 2710 for selection of a next candidate and generation of a next feature set/property set, which is then processed by the machine learning model. This process is repeated until all candidates having probability scores that are above a probability threshold are processed. The probability threshold is reduced, and the process is again repeated until all candidates having probability scores that are above the new probability threshold are processed. This process may be repeated until the probability threshold is reduced to a minimum probability threshold.

Returning again to FIG. 24, at block 2450 processing logic may combine information for the blue spots and the green spots together, and then rerun the candidate selection process of blocks 2425, 2445 but with a combined pool of green and blue spots. At block 2455 a set of 3D points is generated based on the selected candidates. Each selected candidate has an x value, a y value, and a z value, and constitutes a 3D coordinate for a point in a 3D point cloud or intraoral scan.

At block 2460, processing logic may add information for unsolved projector rays from one or more prior frames, adjusting the probability scores for candidates of those projector rays based on distance information for the projector rays in the previous frame(s). After adjusting the probability scores, processing logic may again run the candidate selection algorithms to solve for more candidates. At block 2465 the set of 3D points is updated based on the additional selected candidates.

At the end of method 2400 there may not be solved intersections for all projector rays. Such projector rays with unsolved intersections (and thus unsolved distances/depths) may not be discarded. Enough solutions may be determined to generate a point cloud that has enough properties/features to register and stitch to a 3D surface. Once such registration and stitching is performed, then there may be additional 3D surface data that may be used to help determine the distance/depth information for the previously unsolved projector rays. As further scans are generated and information from those scans is added to the 3D surface, the 3D surface may accumulate data for more points on the surface, further improving the data that can be used to help solve the correspondence problem for previously unsolved projector rays. For example, the 3D coordinates of nearby points may be used to adjust the probability scores for candidates associated with projector rays, after which a candidate selection may be made.

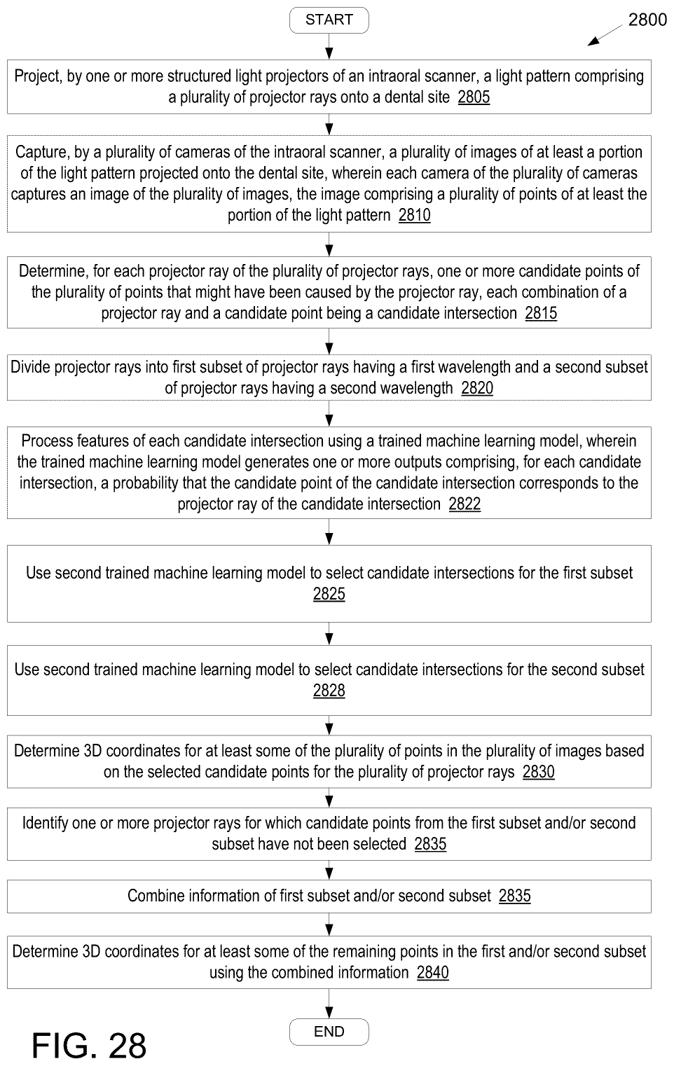

FIG. 28 is a flow chart for a method 2800 of determining 3D coordinates for points in one or more 2D images, where the points include a first set of points generated by light having a first wavelength and a second set of points generated by light having a second wavelength, in accordance with embodiments of the present disclosure. At block 2805 of method 2800, an intraoral scanner is driven to project, using one or more structured light projectors, a pattern of light comprising a plurality of projector rays onto a dental site (each of which causes a projector point when it interfaces with an illuminated surface of the dental site). Though method 2800 is described with regards to two different wavelengths of light, method 2800 may also be performed for systems that emit three or more wavelengths of light.

At block 2810, the intraoral scanner is driven to capture, using one or more cameras, a plurality of images of at least a portion of the light pattern projected onto the dental site. Each camera of the plurality of cameras may capture a distinct image of the dental site at the same time or at approximately the same time, where each of the images comprises a plurality of captured points of at least the portion of the light pattern.

At block 2815, processing logic determines, for each projector ray of the plurality of projector rays of the projected light pattern, one or more candidate points in the images that might have been caused by the projector ray. A candidate intersection may be determined for each pairing of a projector ray and a candidate point of an image.

At block 2820, processing logic divides the projector rays into a first subset of projector rays having a first wavelength and a second subset of projector rays having a second wavelength.

At block 2822, processing logic processes properties/features for each candidate intersection using a trained machine learning model. The trained machine learning model generates one or more outputs containing probability information for each candidate intersection. In one embodiment, different machine learning models are trained for processing candidates for the first and second subsets. A first machine learning model may be trained to process the first subset (e.g., may be trained to process information for rays having the first wavelength) and a second machine learning model may be trained to process the second subset (e.g., may be trained to process information for rays having the second wavelength).

At block 2825, processing logic uses a second trained machine learning model to select candidate intersections for the first subset. At block 2828, processing logic uses a second trained machine learning model to select candidate intersections for the second subset.

At block 2830, processing logic determines depth information (and 3D coordinates) for at least some of the plurality of captured points in the captured images based on the selected candidate intersections.

At block 2835, processing logic may determine one or more projector rays for which candidate points from the first subset and/or second subset have not been selected. At block 2835, processing logic may combine information for the first subset and the second subset. At block 2840, processing logic may then determine 3D coordinates for at least some of the remaining points in the first subset and/or the second subset using the combined information.

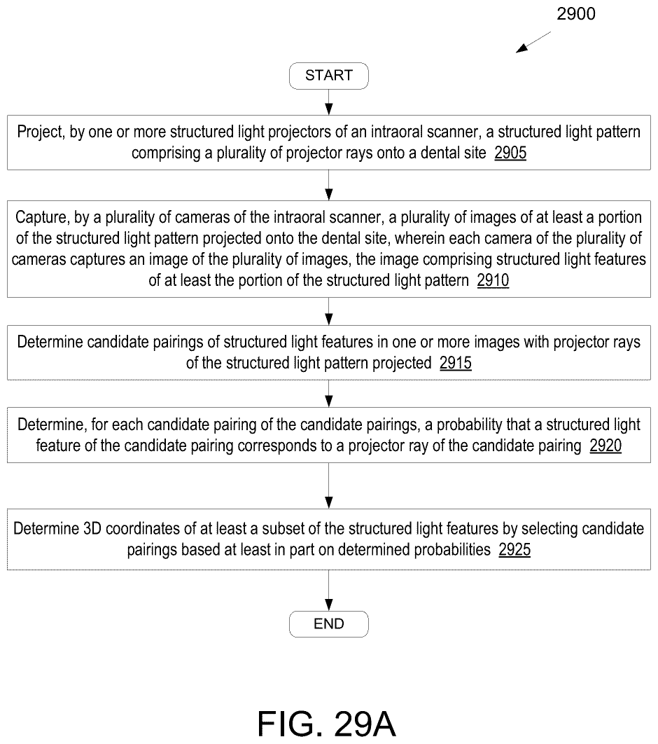

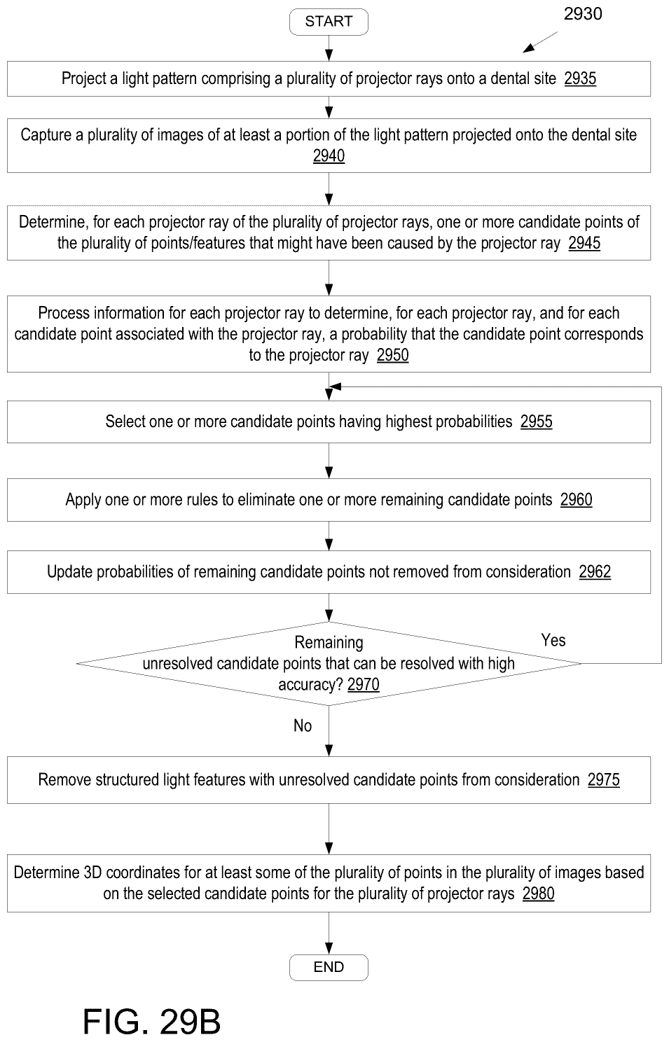

FIG. 29A is a flow chart for a method of determining 3D coordinates for structured light features in one or more 2D images, in accordance with embodiments of the present disclosure. At block 2905 of method 2900, an intraoral scanner is driven to project, using one or more structured light projectors, a structured light pattern comprising a plurality of projector rays onto a dental site. Each of the projector rays causes at least a portion of the structured light pattern (e.g., a point) when it interfaces with an illuminated surface of the dental site. At block 2910, the intraoral scanner is driven to capture, using one or more cameras, a plurality of images of at least a portion of the light pattern projected onto the dental site. Each camera of the plurality of cameras may capture a distinct image of the dental site at the same time or at approximately the same time, where each of the images comprises a plurality of captured points (e.g., captured structured light features) of at least the portion of the light pattern. In one embodiment, the structured light pattern is a pattern of spots, and the plurality of structured light features comprise a plurality of discrete unconnected spots. In one embodiment, the structured light pattern comprises a checkerboard pattern, and the plurality of features comprise a plurality of regions of the checkerboard pattern. In one embodiment, each of the plurality of images is a 2D image.

At block 2915, processing logic determines candidate pairings of structured light features captured in the one or more images with projector rays of the structured light pattern. For each projector ray of the plurality of projector rays of the projected light pattern, processing logic may determine one or more candidate pairings each including a structured light feature (e.g., a point of the structured light pattern in the image) and a projector ray. Such candidate pairings may also be referred to as candidate intersections.

At block 2920, processing logic determines, for each candidate pairing, a probability that the structured light feature of the candidate pairing corresponds to (e.g., was caused by) the projector ray of the candidate pairing. Such probabilities may be determined as described elsewhere herein (e.g., optionally using one or more trained machine learning models and/or based on one or more properties associated with candidate pairings).

At block 2925, processing logic determines 3D coordinates of at least a subset of the structured light features in the images by selecting candidate pairings based at least in part on the determined probabilities. Such selection of candidate pairings (e.g., of candidate points/features) may be determined as described elsewhere herein (e.g., optionally using one or more trained machine learning models). Each candidate pairing represents an intersection of a captured structured light feature with a projector ray on a surface of an imaged object at a known depth or distance from the intraoral scanner. Accordingly, if a captured point/feature is determined to have been caused by a particular projector ray (based on selection of a candidate pairing), then the depth or distance from the intraoral scanner can be determined for the structured light feature of the candidate pairing.

FIG. 29B is a flow chart for a method of determining 3D coordinates for structured light features in one or more 2D images, in accordance with embodiments of the present disclosure. At block 2935 of method 2930, an intraoral scanner is driven to project, using one or more structured light projectors, a structured light pattern comprising a plurality of projector rays onto a dental site. Each of the projector rays causes at least a portion of the structured light pattern (e.g., a point) when it interfaces with an illuminated surface of the dental site. At block 2940, the intraoral scanner is driven to capture, using one or more cameras, a plurality of images of at least a portion of the light pattern projected onto the dental site. Each camera of the plurality of cameras may capture a distinct image of the dental site at the same time or at approximately the same time, where each of the images comprises a plurality of captured points (e.g., captured structured light features) of at least the portion of the light pattern. In one embodiment, the structured light pattern is a pattern of spots, and the plurality of structured light features comprise a plurality of discrete unconnected spots. In one embodiment, the structured light pattern comprises a checkerboard pattern, and the plurality of features comprise a plurality of regions of the checkerboard pattern. In one embodiment, each of the plurality of images is a 2D image.

At block 2945, processing logic determines candidate pairings of structured light features captured in the one or more images with projector rays of the structured light pattern. For each projector ray of the plurality of projector rays of the projected light pattern, processing logic may determine one or more candidate pairings each including a structured light feature (e.g., a point of the structured light pattern in the image) and a projector ray.

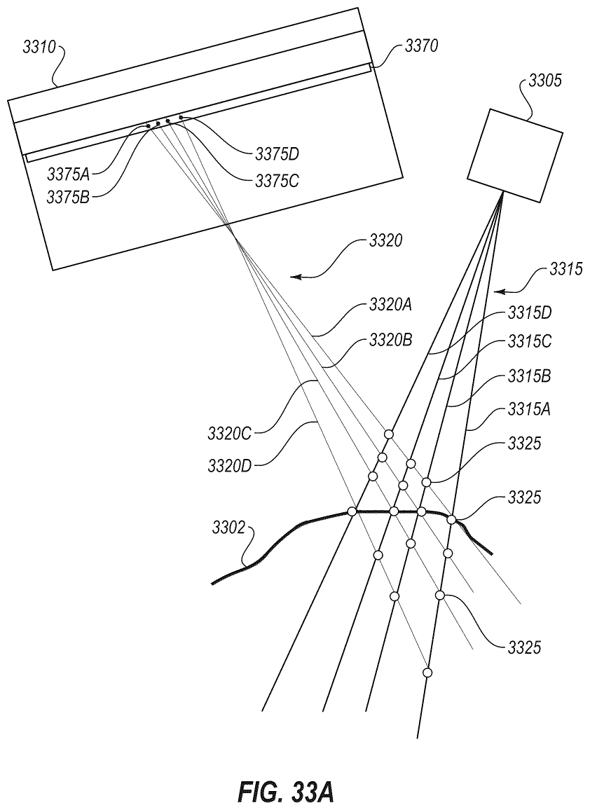

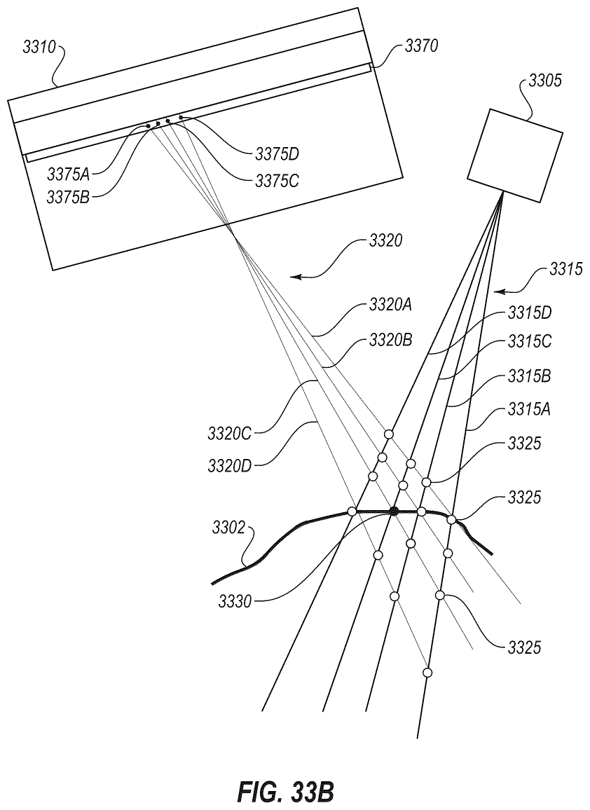

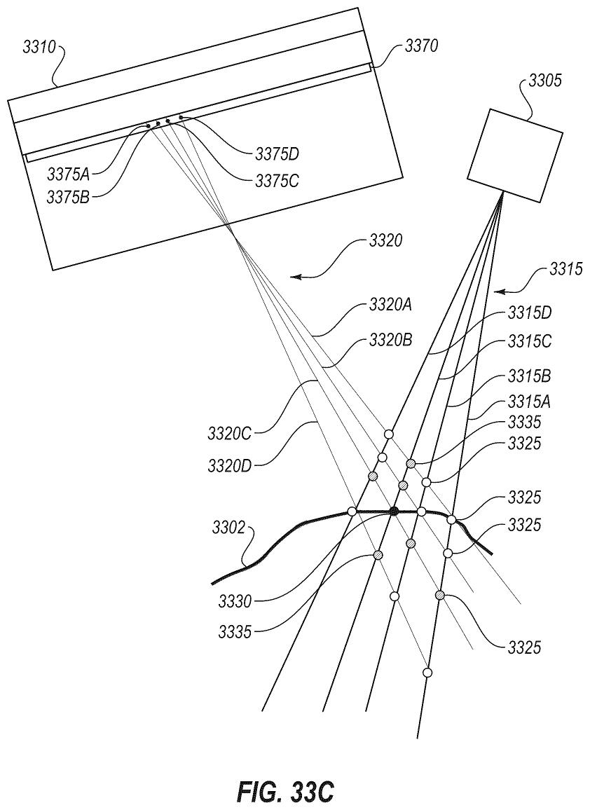

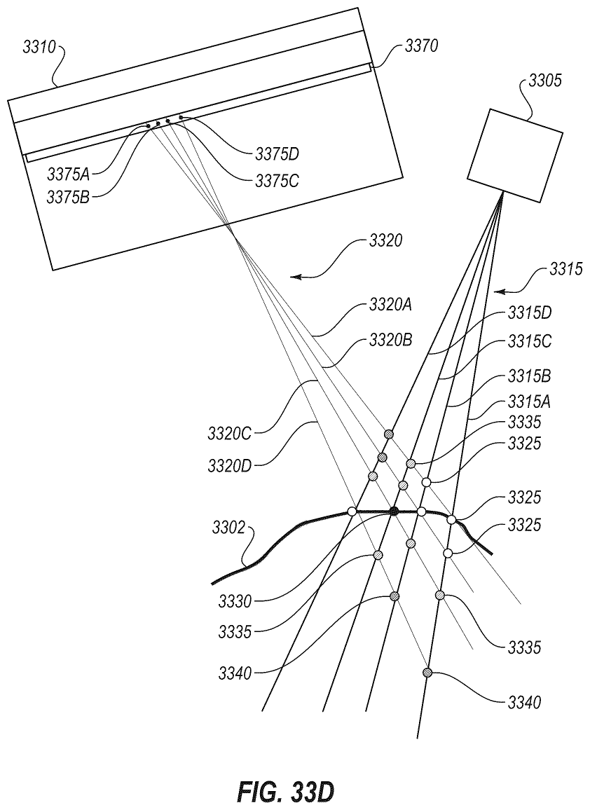

FIGS. 33A-D are schematic illustrations of a structured light projector projecting a structured light pattern and a camera capturing an image of the structured light pattern on an intraoral 3D surface, in accordance with some applications of the present disclosure. FIG. 33A illustrates a structured light projector 3305 of an intraoral scanner that projects a structured light pattern comprising a plurality of projector rays 3315 (e.g., including projector rays 3316A, 3315B, 3315C, 3315D) onto a 3D oral object 3302. FIG. 33A further illustrates a camera 3310 of the intraoral scanner that generates an image 3370 comprising a plurality of structured light features (e.g., points) 3375A, 3375B, 3375C, 3375D, wherein each of the plurality of structured light features 3375A-D captured in the image corresponds to a camera ray 3320A, 3320B, 3320C, 3320D of a plurality of camera rays 3320. The image 3370 captured by the camera 3310 may be a 2D image. Accordingly, the depth value for each of the structured light features 3375A-D may not be immediately apparent. A plurality of candidate intersections 3325 may be determined, where each candidate intersection 3325 is a unique intersection of a camera ray 3320A-D and a projector ray 3315A-D (and corresponds to a unique candidate pairing of a structured light feature 3375A-D associated with a camera ray 3320A-D and a projector ray 3315A-D.

Returning to FIG. 29B, at block 2950, processing logic determines, for each candidate pairing, a probability that the structured light feature of the candidate pairing corresponds to (e.g., was caused by) the projector ray of the candidate pairing. This may include determining, for each projector ray, and for each candidate point (candidate pairing) associated with the projector ray, a probability that the candidate point (candidate pairing) corresponds to the projector ray. Such probabilities may be determined as described elsewhere herein (e.g., optionally using one or more trained machine learning models and/or based on one or more properties associated with candidate pairings).

At block 2955, selects one or more candidate intersections (candidate pairings) having highest probabilities. Such selection of candidate intersections may be determined as described elsewhere herein (e.g., optionally using one or more trained machine learning models).

FIG. 33B illustrates the same scenario as FIG. 33A after a candidate intersection 3330 has been selected. The selected candidate intersection 3330 corresponds to an intersection of projector ray 3315C and camera ray 3320C (and to a candidate pairing of projector ray 3315C and structured light feature 3375C captured in the image 3370).

Once one or more candidate intersection (candidate pairings) are selected, other candidate intersection (candidate pairings) may be eliminated from consideration using one or more rules. For example, each projector ray can cause at most a single point or feature on an imaged object. Similarly, each structured light feature on an imaged object can only be caused by a single projector ray. Accordingly, once a candidate intersection (candidate pairing) is selected for a projector ray, all other candidate intersection (candidate pairings) for the projector ray (e.g., each associated with another possible structured light feature) may be eliminated. Similarly, once a candidate intersection (candidate pairing) is selected for a structured light feature, all other candidate intersection (candidate pairings) for the structured light feature (e.g., associated with other projector rays) may be eliminated.

FIG. 33C illustrates the same scenario as FIG. 33B after candidate intersections 3335 have been eliminated from consideration. As shown, candidate intersections 3335 sharing a same projector ray 3315C as selected candidate intersection 3330 are eliminated from consideration, as are candidate intersection 3335 sharing a same camera ray 3320C as the selected candidate intersection 3330.

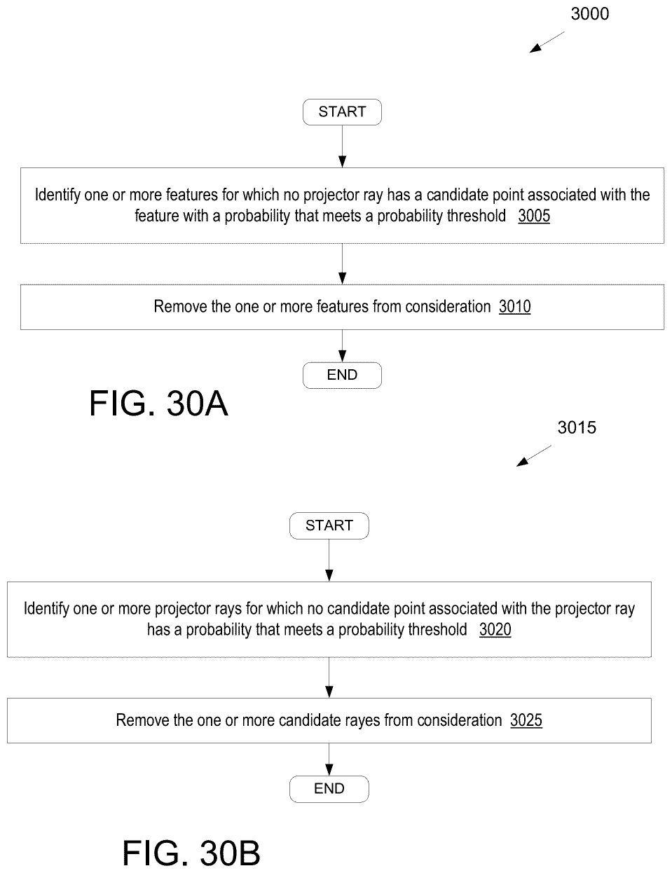

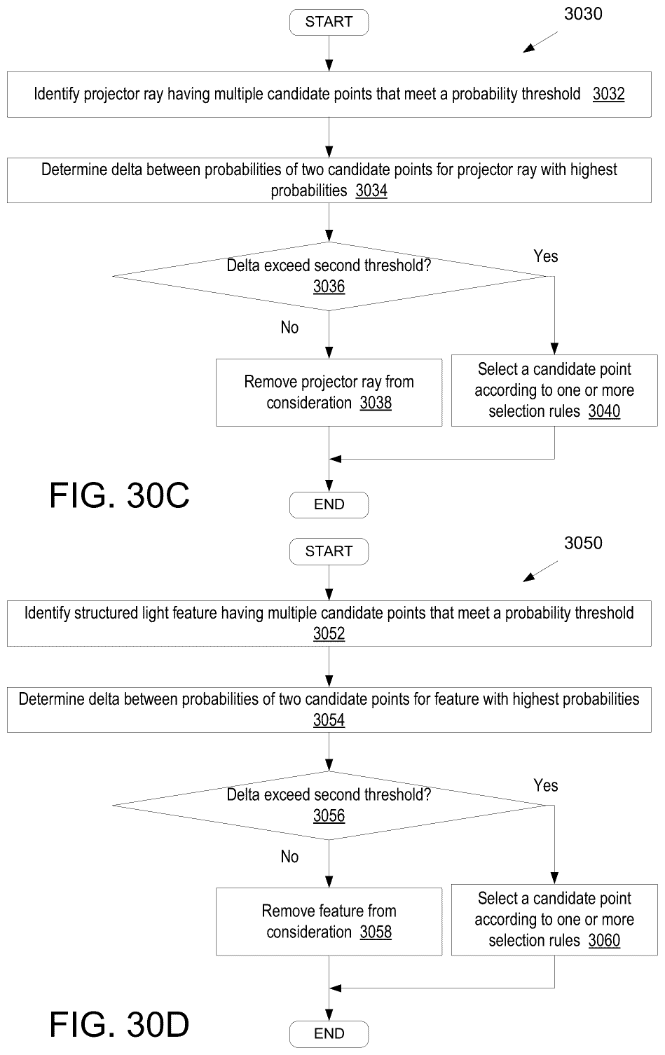

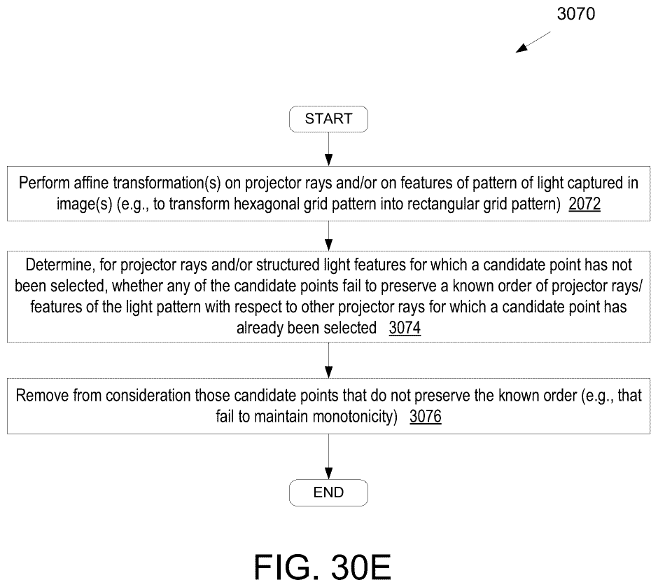

Referring back to FIG. 29B, a rule that may be applied for selection of candidate intersections is that monotonicity is to be maintained. The projector rays of the structured light pattern are projected in a known pattern in which the position and order of each projector ray is known with respect to each other projector ray in the plane (i,j) of the structured light projector. Additionally, the camera and projector are arranged such that the order of the captured structured light features will maintain the known order of the projector rays in a plane (u,v) of the camera. For example, if a first projector ray corresponding to a first structured light feature is to the left of a second projector ray corresponding to a second structured light feature in the projector plane, then the first structured light feature should be to the left of the second structured light feature in the captured image (e.g., in the camera plane). Similarly, if the first projector ray corresponding to the first structured light feature is above the second projector ray corresponding to the second structured light feature in the projector plane, then the first structured light feature should be above the second structured light feature in the captured image (e.g., in the camera plane). This rule is described in greater detail with reference to FIG. 30E.

FIG. 33D illustrates the same scenario as FIG. 33C after candidate intersections 3340 have been eliminated from consideration. Each of the candidate intersections 3340 violated the rule of maintaining monotonicity with respect to selected candidate intersection 3330. As shown, simply by applying two rules, a large number of candidate intersections can be eliminated from consideration. In embodiments, more than two rules are applied, further reducing possible candidate intersections.

Returning to FIG. 29B, at block 2962 probabilities of one or more remaining candidate intersections that have not been removed from consideration are updated. In some embodiments, remaining candidate intersection probabilities are updated by reprocessing information for those candidate intersections using one or more trained machine learning model and/or by reapplying one or more probability assignment rules and/or heuristics (e.g., as described above). Such reprocessing may be performed with additional information on removed candidate intersections in some embodiments. In some embodiments, probabilities are adjusted based on normalizing or otherwise modifying probabilities of remaining candidate intersections to sum up to 100%. This may include determining a combined probability of the removed candidate intersections for a projector ray, and then dividing that combined probability among remaining candidate intersections for the projector ray (e.g., proportionally based on the current probabilities of those candidate intersections). A similar normalization to 100% probability may be performed for the candidate intersections associated with structured light features in some embodiments.

In an example, referring to FIG. 33D, assume for the sake of simplicity that the probability of the candidate intersections for each of the intersection of projector ray 3315D and camera ray 3320A, the intersection of projector ray 3315D and camera ray 3320B, the intersection of projector ray 3315D and camera ray 3320C, and the intersection of projector ray 3315D and camera ray 3320D was initially 25%. Once the candidate intersections for the intersection of projector ray 3315D and camera ray 3320A, the intersection of projector ray 3315D and camera ray 3320B, and the intersection of projector ray 3315D and camera ray 3320C are removed from consideration, and the probability of intersection of projector ray 3315D and camera ray 3320A may be adjusted to 100% or to near 100%. In another example, assume for the sake of simplicity that the probability of the candidate intersections for each of the intersection of projector ray 3315A and camera ray 3320A, the intersection of projector ray 3315A and camera ray 3320B, the intersection of projector ray 3315A and camera ray 3320C, and the intersection of projector ray 3315A and camera ray 3320D was initially 25%. Once the candidate intersections for the intersection of projector ray 3315A and camera ray 3320C and the intersection of projector ray 3315A and camera ray 3320D are removed from consideration, and the probability of intersection of projector ray 3315A and camera ray 3320A and the probability of intersection of projector ray 3315A and camera ray 3320B may be adjusted to 50% each. In another example, assume that the probability of the candidate intersections for the intersection of projector ray 3315A and camera ray 3320A is 40%, the intersection of projector ray 3315A and camera ray 3320B is 30%, the intersection of projector ray 3315A and camera ray 3320C is 20%, and the intersection of projector ray 3315A and camera ray 3320D is 10%. Once the candidate intersections for the intersection of projector ray 3315A and camera ray 3320C and the intersection of projector ray 3315A and camera ray 3320D are removed from consideration, 30% of probability may have been removed from consideration. The remaining candidate intersections may be adjusted by proportionally dividing the unassigned 30% between the intersection of projector ray 3315A and camera ray 3320A and the intersection of projector ray 3315A and camera ray 3320B. This may be accomplished, for example, by dividing each of the remaining probabilities by the sum of all remaining probabilities. For example, 40%/70%=57.14% and 30%/70%=42.86%.

Returning to FIG. 29B, once probabilities of remaining candidate intersections are updated, at block 2970 processing logic determines whether any further candidate intersections have probabilities that are high enough that they can be selected with a high level of confidence. This may include performing any of the previously described selection techniques (e.g., optionally using a machine learning model). If there are additional candidate points that can be selected with a high degree of confidence, the method returns to block 2955 and one or more additional candidate intersections are selected. If there are no remaining candidate intersections (e.g., candidate points or projector rays have been selected for all structured light features), the method may continue to block 2975. Additionally, if the only remaining unassigned candidate intersections cannot be selected with a threshold level of accuracy (e.g., there are structured light features for which a candidate intersection cannot be selected with at least a threshold level of confidence (e.g., 99.9% confidence, 99.5% confidence, 99% confidence, 98% confidence, 95% confidence, etc.), then the method may also proceed to block 2875. Such candidate intersections may be those that fail to satisfy one or more candidate point assignment criteria, as set forth in greater detail with reference to FIGS. 30A-D.

At block 2975 processing logic removes any structured light features with unsolved candidate intersections that cannot be solved with a threshold level of accuracy/confidence from consideration. As a result, data points associated with the structured light features may not be included in a 3D point cloud generated from the 2D image(s). By removing data for structured light features that cannot be solved with a sufficient level of confidence, the accuracy of the 3D point cloud can be increased, resulting in a more accurate 3D model of the scanned dental site generated from the 3D point cloud and other 3D point clouds generated from intraoral scanning. Incorrectly choosing a candidate intersection can reduce a resolution of a 3D point cloud, and ultimately of a generated 3D model of a dental site. Accordingly, it can be beneficial to obtain as clean a point as possible to reproduce an accurate surface. Accordingly, in embodiments points having below a threshold level of confidence or accuracy may be dropped and not included in the 3D point cloud.

At block 2980, processing logic determines 3D coordinates for at least some of the plurality of points (e.g., structured light features) in the images based on the selected candidate intersections for the plurality of projector rays. The 3D coordinates may be used to generate a 3D point cloud.

FIG. 30A is a flow chart for a method 3000 of removing features from consideration for determination of 3D coordinates, in accordance with embodiments of the present disclosure. Method 3000 may be performed, for example, at block 2975 of method 2930 to remove from consideration features having no candidate intersections that can be selected with a threshold level of confidence. At block 3005 of method 3000, processing logic identifies one or more features for which no projector ray has a candidate intersection associated with the feature with a probability that meets a probability threshold. The probability threshold may be, for example 50%, 55%, 60%, 65%, 70%, 75%, 80%, 85%, 90%, 95%, 99%, or some other threshold. At block 3010, processing logic removes the one or more identified features from consideration. Such features will not be included in a generated 3D point cloud, and no 3D coordinates will be determined for such features.

FIG. 30B is a flow chart for a method 3015 of removing projector rays from consideration for determination of 3D coordinates, in accordance with embodiments of the present disclosure. Method 3015 may be performed, for example, at block 2975 of method 2930 to remove from consideration projector rays having no candidate intersections that can be selected with a threshold level of confidence. At block 3020 of method 3015, processing logic identifies one or more projector rays that have no candidate intersections associated with any feature feature with a probability that meets a probability threshold. The probability threshold may be, for example 50%, 55%, 60%, 65%, 70%, 75%, 80%, 85%, 90%, 95%, 99%, or some other threshold. At block 3025, processing logic removes the one or more identified projector rays from consideration.

FIG. 30C is a flow chart for a method 3030 of removing projector rays from consideration for determination of 3D coordinates, in accordance with embodiments of the present disclosure. Method 3030 may be performed, for example, at block 2975 of method 2930 to remove from consideration projector rays having no candidate intersections that can be selected with a threshold level of confidence. At block 3032 of method 3030, processing logic identifies one or more projector rays having multiple candidate intersections that meet a probability threshold (e.g. each of which is approximately equally likely to be correct). At block 3034, for each identified projector ray, processing logic determines a delta between the probabilities of the two candidate intersections for the identified projector ray that have the highest probabilities (e.g., based on subtracting one of the probabilities from the other probability). At block 3036, for each identified projector ray, processing logic determines whether the delta exceeds a second threshold. The second threshold may be a difference threshold. The difference threshold may be, for example, 2%, 5%, 8%, 10%, or some other value. If the delta exceeds the second threshold, the method proceeds to block 3040, and one of the candidate intersections is selected according the one or more of the previously described selection rules. For example, the candidate intersection having the highest probability may be selected. If at block 3036 it is determined that the delta is less than the second threshold, the method continues to block 3038 and the projector ray is removed from consideration. The projector ray may then not be used to determine a 3D coordinate of a structured light feature.

FIG. 30D is a flow chart for a method 3050 of removing features from consideration for determination of 3D coordinates, in accordance with embodiments of the present disclosure. Method 3050 may be performed, for example, at block 2975 of method 2930 to remove from consideration projector rays having no candidate intersections that can be selected with a threshold level of confidence. At block 3052 of method 3050, processing logic identifies one or more structured light features having multiple candidate intersections that meet a probability threshold (e.g. each of which is approximately equally likely to be correct). At block 3054, for each identified structured light feature, processing logic determines a delta between the probabilities of the two candidate intersections for the identified structured light feature that have the highest probabilities (e.g., based on subtracting one of the probabilities from the other probability). At block 3056, for each identified structured light feature, processing logic determines whether the delta exceeds a second threshold. The second threshold may be a difference threshold. The difference threshold may be, for example, 2%, 5%, 8%, 10%, or some other value. If the delta exceeds the second threshold, the method proceeds to block 3060, and one of the candidate intersections is selected according the one or more of the previously described selection rules. For example, the candidate intersection having the highest probability may be selected. Accordingly, the a projector ray is selected for the structured light feature, and a 3D coordinate is determined for the structured light feature. If at block 3056 it is determined that the delta is less than the second threshold, the method continues to block 3058 and the structured light feature is removed from consideration. No 3D coordinate may then be determined for the structured light feature, and it may not be included in a generated 3D point cloud.

FIG. 30E is a flow chart for a method of removing candidate intersections from consideration for determination of 3D coordinates, in accordance with embodiments of the present disclosure. Method 3070 may be performed, for example, at block 2960 of method 2930 to remove from consideration candidate intersections according to one or more candidate intersection removal criteria. As shown in FIG. 18B, each projector may project its own pattern in which each projector ray has a known ordering with respect to each other projector ray. Regardless of the shape of the surface onto which the projector rays are projected, the ordering of the projector rays should be preserved. Accordingly, structured light features in a captured image should correspond to projector rays that cause known ordering to be preserved. In the illustrated example, the structured light pattern is a hexagonal pattern of spots. However, other patterns may alternatively be used, such as a square pattern of spots, a checkerboard pattern, and so on.

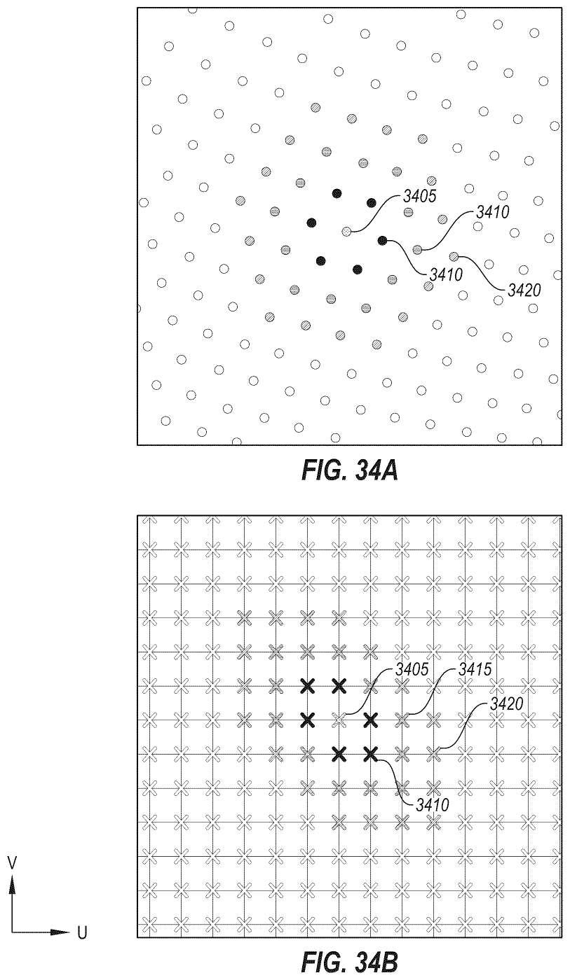

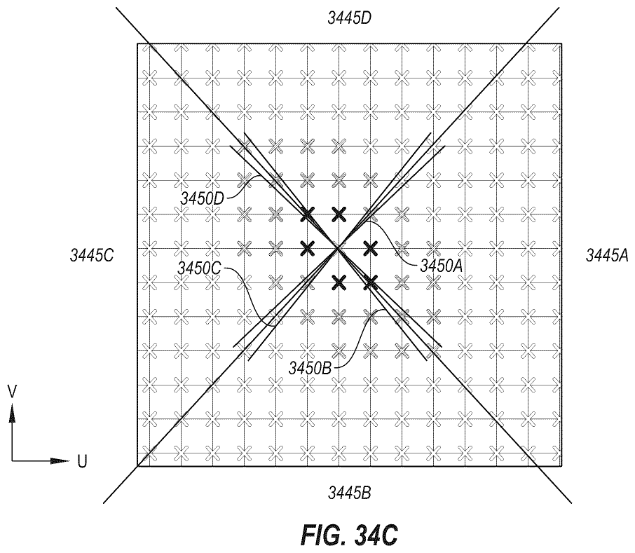

FIG. 34A illustrates an image of a hexagonal pattern of spots of a structured light pattern projected by a projector of an intraoral scanner onto a flat plane, according to one embodiment, where each spot corresponds to a projector ray. For the hexagonal pattern of spots, a given spot/projector ray (e.g., spot 3405) may be solved for, providing 3D coordinates for the given spot. Other spots may be defined relative to the selected spot 3405 based on distance from the selected spot. For example, spots 3410 have a distance of one spot from selected spot 3405, spots 3415 have a distance of two spots from the selected spot 3405, and spots 3420 have a distance of three spots from the selected spot 3405. Each spot should be assigned to an appropriate projector ray that maintains a spatial order of the projector rays in two dimensions. For example, any projector ray (i,j) will always be located on the left side compared to projector ray (i,j+1), and will always be above projector ray (i−1,j). In the extreme case where a surface onto which the structured light pattern is projected is orthogonal to the camera that generates the image, two or more rays may have the same order, but will never swap positions. In embodiments, an intraoral scanner may be calibrated such that the camera to projector orientation for each pair of a camera and a projector is determined that preserves a target u-v:i-j orientation, where u-v are the two axes of the image plane of the camera and i-j are the two axes of the projection plane of the projector.

It can be mathematically complex to determine ordering information of structured light features/projector rays relative to one another for a hexagonal grid pattern. Accordingly, in some embodiments one or more transformations are performed to convert the hexagonal grid pattern into a rectangular grid pattern. It can be easy to determine ordering of structured light features/projector rays in a rectangular grid pattern.

Returning to FIG. 30E, at block 3052 of method 3050, processing logic may perform one or more affine transformations on projector rays and/or on features of the pattern of light captured in one or more images. The affine transformations may be performed to transform a hexagonal grid pattern into a rectangular grid pattern in embodiments. The affine transformation(s) may be performed to cause the camera and projector to be aligned with horizontal and vertical lines. The affine transformations may include one or more shear operations, one or more rotation operations and/or one or more flip operations in embodiments. In embodiments, a first affine transformation may be performed on projector rays of a projector and a second affine transformation may be performed on images of a camera. Different affine transformations may be applied for each camera-projector pair in embodiments, which may be determined based on calibration information on the relative orientations and/or positions of the camera and projector. The transformations may be performed by applying one or more matrix operations in embodiments. Once the affine transformations are performed, ordering information can be determined in x-y coordinates of a transformed image.

FIG. 34B is a schematic illustration of a rectangular grid structured light pattern achieved by performing an affine transformation of the hexagonal grid structured pattern of FIG. 34A, in accordance with some applications of the present disclosure. Each of points 3405, 3410, 3415, 3420 are shown in the x-y coordinate space of the transformed image.

Returning to FIG. 30E, at block 3074 processing logic determines, for projector rays and/or structured light features for which a candidate intersection has not been selected, whether any of the candidate intersections fail to preserve a known order of projector rays/features of the light pattern with respect to other projector rays/features for which a candidate intersection has already been selected. Any candidate intersections that fail to preserve a known order (e.g., fail to preserve monotonicity) with the already solved structured projector ray/structured light feature.

At block 3076, processing logic removes any such candidate intersections that fail to preserve the known order from consideration. Examples of such candidate intersections that fail to preserve a known order are shown in FIG. 33D.

In embodiments, the plurality of projector rays/structured light features are arranged in a first known order along a first axis (e.g., x-axis) and in a second known order along a second axis (e.g., y-axis) in the image plane. Processing logic may eliminate from consideration candidate intersections that fail to preserve either the first known order in the horizontal direction or the second known order in the vertical direction.

In embodiments, once a point (e.g., a structured light feature such as a spot) is solved for, processing logic can eliminate numerous candidate intersections that fail to maintain the correct ordering. In embodiments, up to approximately half or remaining candidate intersections surrounding a solved point may be eliminated based on ordering.

FIG. 34C is a schematic illustration of a rectangular grid structured light pattern in which an image is divided into quadrants about a solved point, in accordance with some applications of the present disclosure.

In some embodiments, each camera may include one or more “confusion” regions, at which it is more difficult to determine whether a known order is preserved. Such confusion regions are regions at which inherent errors may cause the ordering information to be incorrect. The error regions may be one or a few pixels wide in some embodiments. In embodiments, an area around a solved point may be divided into four quadrants 3445A, 3445B, 3445C, 3445D as shown, by drawing diagonal lines through the solved point. Any candidate intersections that fall on or near the line dividing two quadrants may be in a confusion region, and may not be eliminated using ordering information in embodiments. In some embodiments, a confusion region 3450A, 3450B, 3450C, 3450D is a region around each of the lines separating quadrants. In some embodiments, a confusion region is about plus or minus 2.5% or plus or minus 5% around each line dividing quadrants.

In embodiments, ordering information for a candidate intersection may be determined based on the quadrant within which the candidate intersection falls. If the candidate intersection is in quadrant 3445A or 3445C, then ordering information may be determined to maintain ordering in the X axis in an embodiment. If the candidate intersection is in quadrant 3445B or 3445D, then ordering information may be determined to maintain ordering in the Y axis in an embodiment.

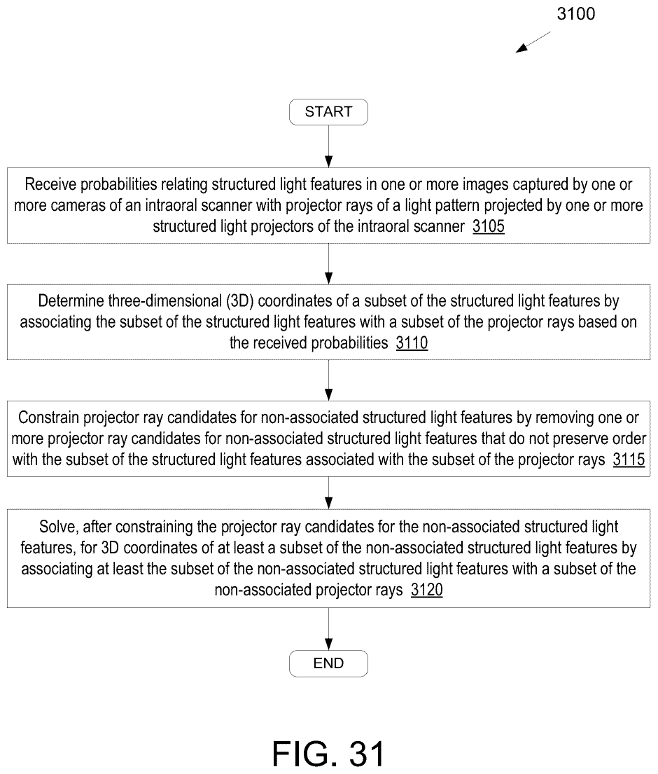

FIG. 31 is a flow chart for a method 3100 of determining 3D coordinates for structured light features in one or more 2D images that applies known ordering of structured light features and/or projector rays to eliminate candidates, in accordance with embodiments of the present disclosure.

At block 3105 of method 3100, processing logic receives probabilities relating structured light features in one or more images captured by one or more cameras of an intraoral scanner with projector rays of a light pattern projected by one or more structured light projectors of the intraoral scanner. The images may have been generated as discussed earlier herein. The probabilities relating structured light features to projector rays may have been determined as described earlier herein (e.g., optionally using machine learning). For example, candidate intersections may be identified, each representing a candidate pairing of a structured light feature and a projector ray, and each candidate intersection may be associated with a probability.

At block 3110, processing logic determines 3D coordinates of a subset of structured light features by associating the subset of the structured light features with a subset of projector rays based on the received probabilities. The 3D coordinates may be determined by selecting candidate intersections that satisfy one or more selection criteria, where each candidate intersection may be associated with a 3D coordinate, as described earlier herein.

At block 3115, processing logic constrains projector ray candidates for non-associated structured light features (e.g., for structured light features for which a candidate intersection has not yet been selected) by removing one or more projector rays candidates (e.g., removing one or more candidate intersections each associated with a different projector ray) for non-associated structured light features that do not preserve order with the subset of the structured light features associated with the subset of the projector rays (e.g., that do not preserve order with an already selected candidate intersection).

At block 3120, processing logic solves, after constraining the projector ray candidates for the non-associated structured light features, for 3D coordinates of at least a subset of the non-associated structured light features by associating at least the subset of the non-associated structured light features with a subset of the non-associated projector rays. Once the projector ray candidates are constrained (e.g., one or more candidate intersections are removed from consideration), the probabilities of remaining projector ray candidates (e.g., remaining candidate intersections) may be increased to a threshold appropriate for selection. 3D coordinates may then be solved for such candidate rays in embodiments.

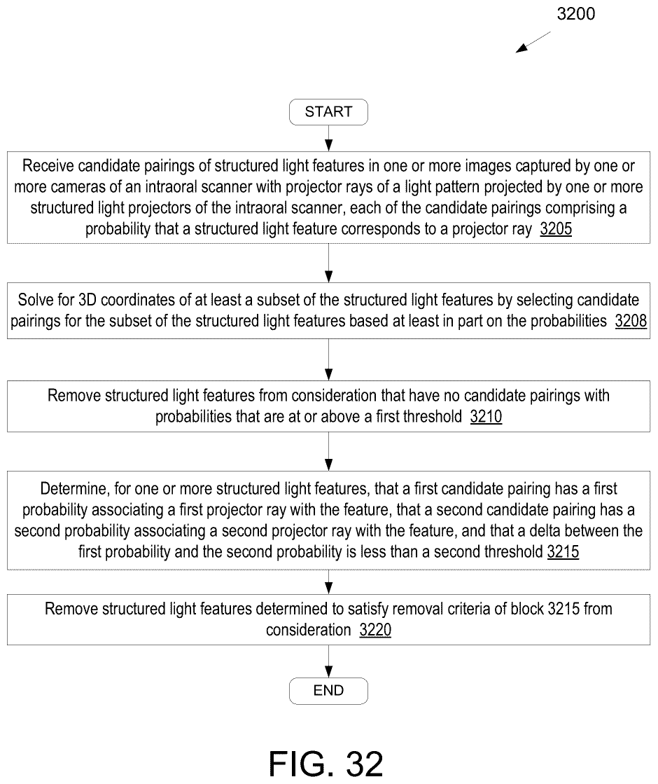

FIG. 32 is a flow chart for a method 3200 of determining 3D coordinates for structured light features in one or more 2D images that eliminates structured light features from consideration that fail to satisfy one or more criteria, in accordance with embodiments of the present disclosure. Method 3200 may be performed, for example, after any of the previously described methods to eliminate one or more features and/or projector rays from consideration that fail to meet certain criteria in some embodiments. In embodiments, method 3200 is performed after all other candidate intersection selection rules and operations have been applied to eliminate from consideration those possibilities that fail to meet a threshold level of confidence.

At block 3205 of method 3200, processing logic receives probabilities relating structured light features in one or more images captured by one or more cameras of an intraoral scanner with projector rays of a light pattern projected by one or more structured light projectors of the intraoral scanner. The images may have been generated as discussed earlier herein. The probabilities relating structured light features to projector rays may have been determined as described earlier herein (e.g., optionally using machine learning). For example, candidate intersections may be identified, each representing a candidate pairing of a structured light feature and a projector ray, and each candidate intersection may be associated with a probability.

At block 3208, processing logic solves for 3D coordinates of at least a subset of the structured light features by associating at least the subset of the structured light features with a subset of projector rays, as discussed earlier herein. For example, processing logic may select candidate intersections for one or more structured light features/points in one or more captured images.

At block 3210, processing logic determines structured light features that have no candidate pairings (e.g., no candidate intersections associating the structured light feature with a projector ray) with probabilities that are at or above a first threshold.

At block 3215, processing logic determines, for one or more structured light features, that a first candidate pairing has a first probability associating a first projector ray with the structured light feature and that a second candidate pairing has a second probability associating a second projector ray with the structured light feature. Processing logic may further determine that the first and second probabilities are each above the first threshold. Processing logic may compare the first and second probabilities to determine a delta between the first probability and the second probability. Processing logic may then determine whether the delta is above a second threshold.

At block 3220, for those structured light features for which the determined delta between probabilities of two possible candidate pairings was less than the second threshold, the structured light features are removed from consideration. This ensures that all solved for 3D coordinates have a very high accuracy (e.g., about 95%, about 99%, about 99.9%, etc.).

FIG. 35 illustrates a diagrammatic representation of a machine in the example form of a computing device 3500 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The computing device 3500 may correspond, for example, to computing device 105 and/or computing device 106 of FIG. 1. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 3500 includes a processing device 3502, a main memory 3504 (e.g., read-only memory (ROM), flash memory, dynamic random access memory (DRAM) such as synchronous DRAM (SDRAM), etc.), a static memory 3506 (e.g., flash memory, static random access memory (SRAM), etc.), and a secondary memory (e.g., a data storage device 3528), which communicate with each other via a bus 3508.

Processing device 3502 represents one or more general-purpose processors such as a microprocessor, central processing unit, or the like. More particularly, the processing device 3502 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 3502 may also be one or more special-purpose processing devices such as an application specific integrated circuit (ASIC), a field programmable gate array (FPGA), a digital signal processor (DSP), network processor, or the like. Processing device 3502 is configured to execute the processing logic (instructions 3526) for performing operations and steps discussed herein.

The computing device 3500 may further include a network interface device 3522 for communicating with a network 3564. The computing device 3500 also may include a video display unit 3510 (e.g., a liquid crystal display (LCD) or a cathode ray tube (CRT)), an alphanumeric input device 3512 (e.g., a keyboard), a cursor control device 3514 (e.g., a mouse), and a signal generation device 3520 (e.g., a speaker).

The data storage device 3528 may include a machine-readable storage medium (or more specifically a non-transitory computer-readable storage medium) 3524 on which is stored one or more sets of instructions 3526 embodying any one or more of the methodologies or functions described herein, such as instructions for intraoral scan application 3515, which may correspond to intraoral scan application 115 of FIG. 1. A non-transitory storage medium refers to a storage medium other than a carrier wave. The instructions 3526 may also reside, completely or at least partially, within the main memory 3504 and/or within the processing device 3502 during execution thereof by the computing device 3500, the main memory 3504 and the processing device 3502 also constituting computer-readable storage media.

The computer-readable storage medium 3524 may also be used to store dental modeling logic 3550, which may include one or more machine learning modules, and which may perform the operations described herein above. The computer readable storage medium 3524 may also store a software library containing methods for the intraoral scan application 115. While the computer-readable storage medium 3524 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1.-81. (canceled)

82. An intraoral scanning system, comprising:
an intraoral scanner comprising one or more structured light projectors configured to output a light pattern and one or more cameras configured to generate a plurality of images of a dental site illuminated by the light pattern; and
a computing device configured to:
determine candidate pairings of structured light features in one or more images of the plurality of images with projector rays the a light pattern projected by the one or more structured light projectors of the intraoral scanner;
determine, for each candidate pairing of the candidate pairings, a probability that a structured light feature of the candidate pairing corresponds to a projector ray of the candidate pairing; and
determine 3D coordinates of at least a subset of the structured light features by selecting candidate pairings based at least in part on determined probabilities.

83. The intraoral scanning system of claim 82, wherein the computing device is further configured to:
remove one or more candidate pairings for which a known order of structured light features is not preserved; and
solve for 3D coordinates of one or more additional structured light features by selecting one or more remaining candidate pairings for the one or more additional structured light features.

84. The intraoral scanning system of claim 82, wherein the computing device is further to:
remove structured light features from consideration that have no candidate pairings with probabilities that are at or above a threshold.

85. The intraoral scanning system of claim 82, wherein the computing device is further to:
determine, for a feature of the structured light features, that a first candidate pairing has a first probability associating a first projector ray with the feature, that a second candidate pairing has a second probability associating a second projector ray with the feature, and that a delta between the first probability and the second probability is less than a threshold; and
remove the feature from consideration, wherein no 3D coordinate is solved for the feature.

86. The intraoral scanning system of claim 82, wherein determining a probability that a structured light feature of a candidate pairing corresponds to a projector ray of the candidate pairing comprises:
processing information for the candidate pairing using a trained machine learning model, wherein the trained machine learning model generates an output comprising the probability that the structured light feature corresponds to the projector ray.

87. The intraoral scanning system of claim 82, wherein the computing device is further to:
select the candidate pairings based at least in part on the determined probabilities by processing one or more inputs comprising one or more of the determined probabilities using a trained machine learning model, wherein the trained machine learning model outputs one or more selections of candidate pairings.

88. An intraoral scanning system, comprising:
an intraoral scanner comprising:
one or more structured light projectors configured to project a light pattern comprising a plurality of projector rays onto a dental site; and
a plurality of cameras configured to capture a plurality of images of at least a portion of the light pattern projected onto the dental site, wherein each camera of the plurality of cameras is configured to capture an image of the plurality of images, the image comprising a plurality of points of at least the portion of the light pattern projected onto the dental site; and
a computing device configured to:
determine, for each projector ray of the plurality of projector rays, one or more candidate points of the plurality of points that might have been caused by the projector ray;
process information for each projector ray using a trained machine learning model, wherein the trained machine learning model generates one or more outputs comprising, for each projector ray, and for each candidate point associated with the projector ray, a probability that the candidate point corresponds to the projector ray; and
determine three-dimensional (3D) coordinates for at least some of the plurality of points in the plurality of images based on the one or more outputs of the trained machine learning model.

89. The intraoral scanning system of claim 88, wherein the computing device is further configured to:
determine, for each projector ray, and for each candidate point of the one or more candidate points that might have been caused by the projector ray, a distance at which the candidate point intersects with the projector ray, wherein the information for the projector ray that is input into the trained machine learning model comprises the distance.

90. The intraoral scanning system of claim 89, wherein the computing device is further configured to:
for each projector ray, group one or more candidate points from different images of the plurality of images for which the distance matches into a candidate intersection, wherein the candidate intersection comprises an intersection of the one or more candidate points with the projector ray, and wherein the distance for candidate points match if the distance varies by less than a threshold amount.

91. The intraoral scanning system of claim 90, wherein the computing device is further configured to:
determine, for each candidate intersection, a triangulation point of the candidate intersection, wherein the information for the projector ray that is input into the trained machine learning model comprises the triangulation point.

92. The intraoral scanning system of claim 89, wherein the one or more structured light projectors comprise a plurality of structured light projectors, and wherein the computing device is further configured to:
determine, for each projector ray, an index of a structured light projector of the plurality of structured light projectors that generated the projector ray, wherein the information for the projector ray that is input into the trained machine learning model comprises the index.

93. The intraoral scanning system of claim 92, wherein a first subset of the plurality of structured light projectors produces light having a first wavelength, and wherein a second subset of the plurality of structured light projectors produces light having a second wavelength, and wherein the computing device is further configured to:
determine the 3D coordinates for one or more points of a first subset of points of the plurality of points having the first wavelength; and
independently determine the 3D coordinates for one or more additional points of a second subset of points of the plurality of points having the second wavelength;
identify one or more projector rays for which candidate points from at least one of the first subset of points or the second subset of points have not been selected;
combine information for the first subset of points and the second subset of points; and
determine the 3D coordinates for one or more additional points of the first subset of points and the 3D coordinates for one or more additional points of the second subset of points after combining the information.

94. The intraoral scanning system of claim 89, wherein the computing device is further configured to:
determine, for each projector ray, and for one or more candidate points associated with the projector ray, one or more features associated with the projector ray and the one or more candidate points, wherein the information for the projector ray that is input into the trained machine learning model comprises the one or more features, and wherein the one or more features comprise at least one of:
a distance from an epi-polar line associated with the projector ray;
for an image associated with a candidate point, a triangulation error that is determined based on a distance between a camera that captured the image and an origin of the projector ray;
an intensity associated with a captured point;
a spot size of the captured point; or
a color of the dental site at the intersection of a candidate point with the projector ray as determined from one or more color images captured at least one of before or after capture of the plurality of images.

95. The intraoral scanning system of claim 88, wherein the computing device is further configured to:
generate a tuple for a projector ray comprising:
distances and probabilities for one or more top candidate points for the projector ray; and
distances and probabilities for one or more top candidate points for one or more additional projector rays that are proximate to the projector ray; and
input the tuple into a second trained machine learning model, wherein the second trained machine learning model outputs an updated probability for one or more candidate points for the projector ray.

96. The intraoral scanning system of claim 88, wherein the plurality of images are associated with a current frame, wherein a previous plurality of images was generated at a prior frame prior to generation of the plurality of images, and wherein the computing device is further configured to:
determine, for a projector ray, a 3D coordinate associated with the projector ray for the prior frame; and
update, for a candidate point for the projector ray, the probability that the candidate point corresponds to the projector ray based on the 3D coordinate associated with the projector ray for the prior frame.

97. The intraoral scanning system of claim 88, wherein the computing device is further configured to:
use a second trained machine learning model to select candidate points for a plurality of projector rays based on one or more inputs comprising probabilities of candidate points corresponding to projector rays, wherein the 3D coordinates are determined based on the selected candidate points.

98. An intraoral scanning system, comprising:
an intraoral scanner comprising:
one or more structured light projectors configured to project a light pattern comprising a plurality of projector rays onto a dental site; and
a plurality of cameras configured to capture a plurality of images of at least a portion of the light pattern projected onto the dental site, wherein each camera of the plurality of cameras is configured to capture an image of the plurality of images, the image comprising a plurality of points of at least the portion of the light pattern projected onto the dental site; and
a computing device configured to:
determine, for each projector ray of the plurality of projector rays, one or more candidate points of the plurality of points that might have been caused by the projector ray, each candidate point of the one or more candidate points having a determined probability of corresponding to the projector ray;
use a trained machine learning model to select candidate points for a plurality of projector rays based on one or more inputs comprising probabilities of candidate points corresponding to projector rays; and
determine three-dimensional (3D) coordinates for at least some of the plurality of points in the plurality of images based on the selected candidate points for the plurality of projector rays.

99. The intraoral scanning system of claim 98, wherein the computing device is further configured to:
generate an input comprising a candidate point for a projector ray, one or more additional candidate points for the projector ray, and one or more additional projector rays for the candidate point;
provide the input to the trained machine learning model, wherein the trained machine learning model outputs a selection of the candidate point or one of the one or more additional candidate points for the projector ray; and
remove the selected candidate point from association with the one or more additional projector rays that were associated with the selected candidate point.

100. The intraoral scanning system of claim 99, wherein the computing device is further configured to:
repeatedly perform the following until no remaining projector rays have an associated candidate point with at least a threshold probability:
generate a next input comprising a next candidate point for a next projector ray, one or more next additional candidate points for the next projector ray, and one or more next additional projector rays for the next candidate point;
provide the next input to the trained machine learning model, wherein the trained machine learning model outputs a selection of the next candidate point or one of the one or more next additional candidate points for the next projector ray; and
remove the selected next candidate point from association with the one or more next additional projector rays that were associated with the selected next candidate point; and

101. The intraoral scanning system of claim 98, wherein the computing device is further configured to:
generate a first list associating projector rays with candidate intersections, wherein each candidate intersection comprises an intersection of a projector ray of the plurality of projector rays and a candidate point of the one or more candidate points that might have been caused by the projector ray, the first list comprising, for each projector ray, one or more candidate intersections associated with the projector ray; and
generate a second list of the plurality of points, the second list comprising, for each point of the plurality of points, one or more candidate intersections associated with the point;
wherein at least one of the first list or the second list is used to generate the input.
